(19) United States
(12) Patent Application Publication
(10) Pub. No.: US 2024/0308999 A1
(43) Pub. Date: Sep. 19, 2024

(54) INDOLIZINE COMPOUNDS FOR THE TREATMENT OF MENTAL DISORDERS OR MENTAL ENHANCEMENT

(71) Applicant: TACTOGEN INC, Palo Alto, CA (US)

(72) Inventor: Matthew J. Baggott, Redwood City, CA (US)

(73) Assignee: TACTOGEN INC, Palo Alto, CA (US)

(21) Appl. No.: 18/653,750

(22) Filed: May 2, 2024

Related U.S. Application Data
(63) Continuation of application No. PCT/US2022/048867, filed on Nov. 3, 2022.
(60) Provisional application No. 63/275,324, filed on Nov. 3, 2021.

(51) Int. Cl. C07D 471/04; A61K 31/437; A61P 25/22

(52) U.S. Cl. CPC C07D 471/04; A61K 31/437; A61P 25/22

(57) ABSTRACT

The present invention discloses a compound, composition, method for modulating central nervous system activity, or method for treating central nervous system disorders, such as post-traumatic stress and adjustment disorders, comprising an indolizine-containing compound having a structure disclosed herein.

### CROSS-REFERENCE TO RELATED APPLICATIONS

This application is a continuation of International Patent Application No. PCT/US2022/048867, filed in the U.S. Receiving Office on Nov. 3, 2022, which claims the benefit of U.S. Provisional Application 63/275,324 filed on Nov. 3, 2021. The entirety of each of these applications is hereby incorporated by reference herein for all purposes.

### FIELD OF THE INVENTION

This invention is in the area of pharmaceutically active indolizine compounds and compositions for the treatment of mental disorders or for mental enhancement, including for entactogenic therapy. The present invention also includes indolizine compounds, compositions, and methods for modulating central nervous system activity and treating central nervous system disorders.

### BACKGROUND

Central nervous system (CNS) related health problems are a common challenge in society. An estimated 20.6% of U.S. adults (51.5 million people) experienced mental illness in 2019. This includes major depression (7.8% or 19.4 million people), anxiety disorders (19.1% or 48 million people), and post-traumatic stress disorder (PTSD) (3.6% or 9 million people). In addition to mental health challenges, there are other CNS disorders that cause substantial suffering and decreased quality of life. These include traumatic brain injury (TBI) (an estimated 12% of adults or 30 million people), dementias, and headache disorders (such as migraine, which affects about 15% of the general population or 47 million people). As the global population ages, many age-related CNS disorders are projected to become more common. For example, 6.2 million people aged 65 and older in the U.S. have Alzheimer's dementia and this population is expected to grow to 12.7 million by 2050.

There is a need for improved treatment of CNS disorders. Many patients fail to benefit adequately from available treatments. In addition, many available pharmacological treatments must be taken for weeks or months before the individual experiences therapeutic benefits.

A number of potential new experimental treatments are under investigation. These include compounds that modulate the functioning of the monoamine neurotransmitters, dopamine, norepinephrine, and serotonin. Dopamine is involved in learning, incentives, and the initiation of motor movements. Norepinephrine is important for attention and cardiovascular functioning. Serotonin is incompletely understood but appears to adjust the stability of the individual's response to changing environmental conditions. As such, serotonin has been linked to mood, anxiety, and appetite.

Experimental treatment compounds include serotonin receptor agonists. Serotonin receptors have seven families. Many serotonin receptors are able to stimulate multiple signaling pathways within a cell, which can make it complicated to predict therapeutic effects. Serotonin receptor types that have received recent attention for their therapeutic potential include 5-HT_{2A}, 5-HT_{2C}, 5-HT_{1A}, and 5-HT_{1B} receptors.

One group of experimental therapeutic compounds are 5-HT_{2A} receptor agonists. These are being investigated as tools to potentially produce rapid therapeutic improvement in CNS disorders including depression, anxiety, and substance use disorders. Compounds such as psilocybin and 5-methoxy-N,N-dimethyltryptamine (5-MeO-DMT), produce dramatic psychedelic effects resembling mystical experiences that may contribute to these therapeutic effects. These compounds also produce labile mood and often invoke acute anxiety, which makes close monitoring of patients necessary. There is accordingly a need for 5-HT_{2A} agonists that produce either minimal mood changes or reliably positive ones.

Indeed, another group of putative 5-HT_{2A} agonists, such as 6-methoxy-N,N-dimethyltryptamine (6-MeO-DMT) and 7-fluoro-N,N-dimethyltryptamine (7-F-DMT) appear to produce therapeutic changes in animal models of depression without producing psychedelic effects (Dunlap et al. 2020. Journal of medicinal chemistry, 63(3), pp. 1142-1155). Both psychedelic and non-psychedelic 5-HT_{2A} agonists may be useful in migraine, cluster headaches, and other headache disorders.

The therapeutic mechanisms of 5-HT_{2A} agonists are incompletely understood but may involve increased neuroplasticity (Ly et al. 2018. Cell reports, 23(11), pp. 3170-3182), suggesting potential benefits in TBI, neurological disorders, and conditions where behavior change or learning is desired.

Another potential therapeutic mechanism of 5-HT_{2A} agonists involves decreases in inflammation (e g., Flanagan, et al. 2019. Life sci., 236, 116790). Conditions that may benefit from improved anti-inflammatory treatment include rheumatoid and other forms of arthritis (such as enthesitis-related juvenile idiopathic arthritis, blau syndrome, and juvenile idiopathic arthritis), psoriasis, Crohn's disease, inflammatory bowel syndrome, ulcerative colitis, and ankylosing spondylitis. Inflammation has long been recognized to induce symptoms of depression (Lee & Giuliani. 2019. Frontiers in immunology, 10, 1696). Inflammatory processes have also been implicated in psychotic disorders (Borovcanin et al. 2012. J. Psych. Res., 46(11), 1421-1426) and bipolar disorders (Hamdani, Tamouza, & Leboyer. 2012. Front. Biosci. (Elite Ed.), 4, 2170-2182).

5-HT_{2A} agonists are also often 5-HT_{2B} agonists. This is undesirable because chronic stimulation of 5-HT_{2B} receptors causes cardiac valvopathy (Rothman et al. 2000. Circulation, 102(23), pp. 2836-2841). There is therefore a need for serotonin agonists that have decreased ability to stimulate 5-HT_{2B} receptors.

5-HT_{2C} receptors are closely related to 5-HT_{2A} receptors, but have a different distribution in the brain and body. Compounds that stimulate 5-HT_{2C} receptors have been proposed as treatments for psychiatric disorders as well as other disorders such as sexual dysfunction, obesity, and urinary incontinence. Lorcaserin (Belviq) is a high affinity 5-HT_{2C} agonist that, until recently, was FDA-approved for use in conjunction with weight loss programs. The withdrawal of this medicine from the market because of increased risk of cancer highlights the need for safer serotonergic therapeutics that can stimulate 5-HT_{2C} receptors or otherwise aid weight loss.

5-HT_{1A} receptor agonists modulate the functioning of dopamine and norepinephrine and decrease blood pressure and heart rate via a central mechanism. Drugs that are 5-HT_{1A} agonists have value for treating anxiety and depression. For example, buspirone (Buspar, Namanspin) is approved for anxiety disorders and may also be useful for treating hypoactive sexual desire disorder (HSDD). Studies in rats indicate that 5-HT_{1A} stimulation induces oxytocin release, which contributes to the social effects of 3,4-methylenedioxymethamphetamine (MDMA) (Thompson et al. 2007. Neuroscience, 146(2), pp. 509-514). Compounds (or compound combinations) that include 5-HT_{1A} stimulation in their pharmacological profile are therefore expected to have therapeutic benefits in comparison to those that do not.

Compounds that stimulate 5-HT_{1B} receptors alter the release of neurotransmitters such as dopamine, serotonin, GABA, acetylcholine, and glutamate and can modulate stress sensitivity, mood, anxiety, and aggression. 5-HT_{1B} agonists such as sumatriptan (Imitrex) and zolmitriptan (Zomig) have been approved for treatment of headache disorders. Studies in mice suggest 5-HT_{1B} stimulation on dopamine-containing neurons in the central striatum contributes to social effects of MDMA (Heifets et al. 2019. Science translational medicine, 11(522)). Preclinical studies also suggest 5-HT_{1B} agonists may have antidepressant effects. More broadly, there is evidence that stimulating 5-HT_{1B} receptors can provide benefits to stress response, affect, and addiction (e.g., Fontaine et al. 2021. Neuropsychopharmacology, pp. 1-11). As with 5-HT_{1A} receptors, compounds (or compound combinations) that include 5-HT_{1B} stimulation in their pharmacological profile are therefore expected to have therapeutic benefits in comparison to those that do not.

Another group of experimental compounds interact with brain monoamine transporters to increase extracellular concentrations of the three monoamine neurotransmitters. Some compounds increase extracellular concentrations of these molecules by inhibiting reuptake of neurotransmitters, while others induce release of neurotransmitters.

Patent applications describing entactogenic compounds include WO 2021/252538, WO 2022/010937, WO 2022/032147, and WO 2022/061242 which are assigned to Tactogen Inc. Additional patent applications describing entactogenic compounds and methods of using entactogenic compounds include but are not limited to U.S. Pat. No. 7,045,545, WO 2005/058865, WO 2020/169850, WO 2020/169851, WO 2021/257169, WO 2021/225796, WO 2022/214889, WO 2022/120181, WO 2022/072808, and WO 2022/038171.

Despite the ongoing research on potential new drugs to treat mental disorders, CNS disorders, and related gastrointestinal and inflammatory disorders, the enormous burden of disease caused by these disorders remains a global serious and systemic problem. New drugs and treatments are required to improve personal well-being, mental health, and physical health that are dependent on the alteration of neurotransmitter levels and performance.

It is therefore an object of the present invention to provide advantageous compositions and their use and manufacture for the treatment of mental disorders and enhancement for hosts, typically humans, in need thereof. Additional objects are to provide drugs with an efficient onset to be used in a clinical setting such as counseling or a home setting, which open the patient to empathy, sympathy and acceptance. A further object is to provide effective treatments for a range of CNS disorders.

### SUMMARY OF THE INVENTION

The present invention provides advantageous indolizine compounds and their pharmaceutically acceptable salts and salt mixtures thereof, pharmaceutical compositions, and methods to treat mental disorders and more generally central nervous system and related disorders as described herein. An indolizine compound of the present invention can be used for mental enhancement or to treat a mental disorder comprising administering an effective amount of the compound to a host, typically a human, in need thereof. In some embodiments, the indolizine compounds or compositions described herein interact with a serotonergic binding site and can exhibit entactogenic properties when administered in an effective amount to a host, typically a human, in need thereof. Thus, a compound described herein can be used as an effective agent for modulating CNS activity and treating CNS disorders described herein.

In certain aspects, the invention provides a compound of Formula I

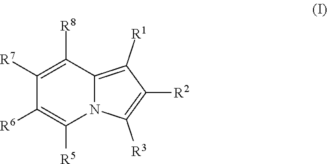

or a pharmaceutically acceptable salt or salt mixture thereof and its use to treat the described disorders;
wherein:
R² is selected from R¹,

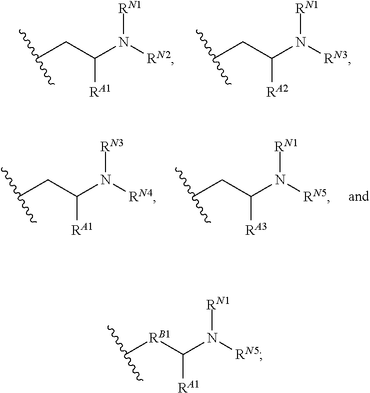

R³ is selected from R¹,

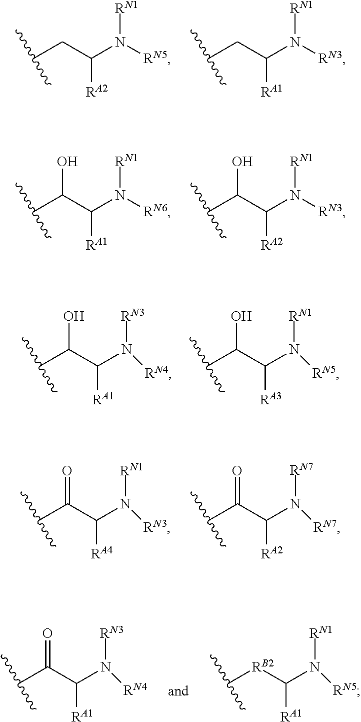

R⁵, R⁶, R⁷, and R⁸ are independently selected from R¹ and

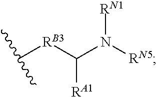

wherein 5 of the 6 of R², R³, R⁵, R⁶, R⁷, and R⁸ are R¹;
and wherein each R¹ is independently selected from the group consisting of hydrogen, halogen, alkyl, haloalkyl, —OP(O)(OR⁹)₂, —SR⁹, —NR⁹R¹⁰, and —OR⁹;
in certain embodiments 1, 2, 3, 4, 5, or 6 R¹ groups are hydrogen;
each R⁹ and R¹⁰ is independently selected from the group consisting of hydrogen, alkyl, and haloalkyl;
R^{A1} is selected from hydrogen, —CH₃, —CH₂X, —CHX₂, —CX₃, —CH₂CH₃, —CH₂CH₂X, —CH₂CHX₂, —CH₂CX₃, —CH₂OH, and —CH₂CH₂OH;
R^{A2} is selected from —CH₃, —CH₂X, —CHX₂, —CX₃, —CH₂CH₃, —CH₂CH₂X, —CH₂CHX₂, —CH₂CX₃, —CH₂OH, and —CH₂CH₂OH;
R^{A3} is selected from —CH₂X, —CHX₂, —CX₃, —CH₂CH₃, —CH₂CH₂X, —CH₂CHX₂, —CH₂CX₃, —CH₂OH, and —CH₂CH₂OH;
R^{A4} is selected from hydrogen, —CH₂X, —CHX₂, —CX₃, —CH₂CH₃, —CH₂CH₂X, —CH₂CHX₂, —CH₂CX₃, —CH₂OH, and —CH₂CH₂OH;
R^{B1} is selected from

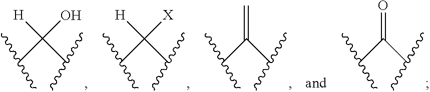

R^{B2} is selected from

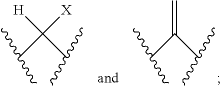

R^{B3} is selected from

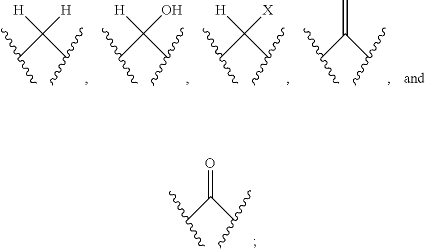

R^{N1} is independently selected in each instance from hydrogen, —(C₁-C₆)alkyl, —CH₂CH₂X, —CH₂CHX₂, —CH₂CX₃, and —CH₂CH₂OH;
R^{N2} is selected from —(C₃-C₆)alkyl, —CH₂CH₂X, —CH₂CHX₂, —CH₂CX₃, —CH₂CH₂OH, and hydroxy;
R^{N3} is selected from —(C₁-C₆)alkyl, —CH₂CH₂X, —CH₂CHX₂, —CH₂CX₃, —CH₂CH₂OH, and hydroxy;
R^{N4} is selected from —(C₁-C₆)alkyl, —CH₂CH₂X, —CH₂CHX₂, —CH₂CX₃, and —CH₂CH₂OH;
R^{N5} is independently selected in each instance from hydrogen, —(C₁-C₆)alkyl, —CH₂CH₂X, —CH₂CHX₂, —CH₂CX₃, —CH₂CH₂OH, and hydroxy;
R^{N6} is selected from —(C₂-C₆)alkyl, —CH₂CH₂X, —CH₂CHX₂, —CH₂CX₃, —CH₂CH₂OH, and hydroxy;
R^{N7} is independently selected in each instance from hydrogen, —(C₂-C₆)alkyl, —CH₂CH₂X, —CH₂CHX₂, —CH₂CX₃, —CH₂CH₂OH, and hydroxy; and
X is independently in each instance selected from —F, —Cl, —Br, and —I.

In certain embodiments the compound of Formula I is of Formula Ia:

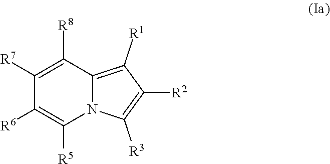

or a pharmaceutically acceptable salt or salt mixture thereof;
wherein R¹ is hydrogen.

The carbon alpha to the amine is chiral when R^{A} is not hydrogen. The invention includes a compound of either the R- or S-stereochemistry at this carbon. An isolated R- or S-enantiomeric compound of the present invention can be used as a pure enantiomer or combined with the other enantiomer in any ratio that produces the desired effects.

In certain embodiments the compound of Formula I or Formula Ia is a racemate.

In certain embodiments the compound of Formula I or Formula Ia is a pure R- or S-enantiomer.

In certain embodiments the compound of Formula I or Formula Ia is an enantiomerically enriched mixture or R- and S-enantiomers.

Exemplary compounds of the present invention include the racemic compounds Structure I and Structure II, the pure S- or R-enantiomers of Structures Ia, Ib, IIa, and IIb, or enantiomerically enriched mixtures of Structures Ia, Ib, IIa, and IIb:

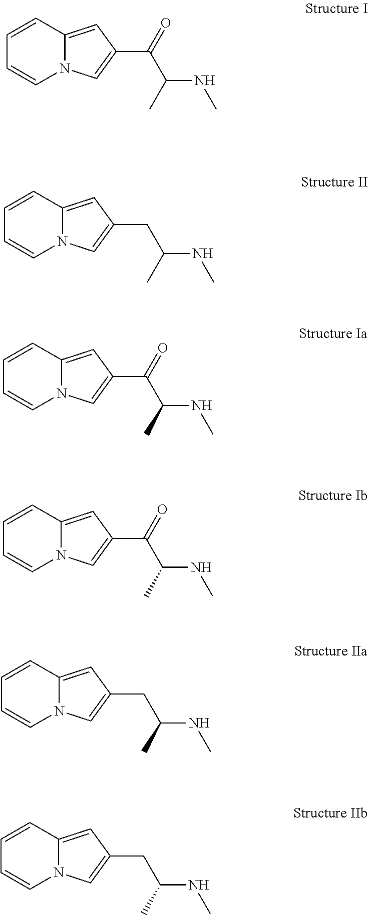

In other aspects of the invention, a compound of Formula II is provided:

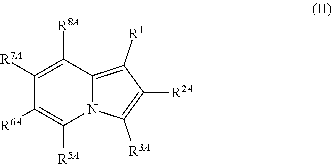

or a pharmaceutically acceptable salt or salt mixture thereof;
wherein:
R^{2A} is selected from R¹,

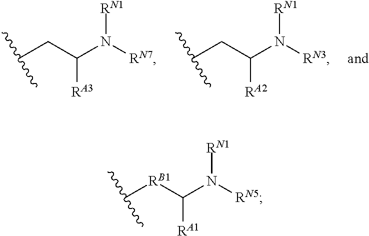

R^{3A} is selected from R¹,

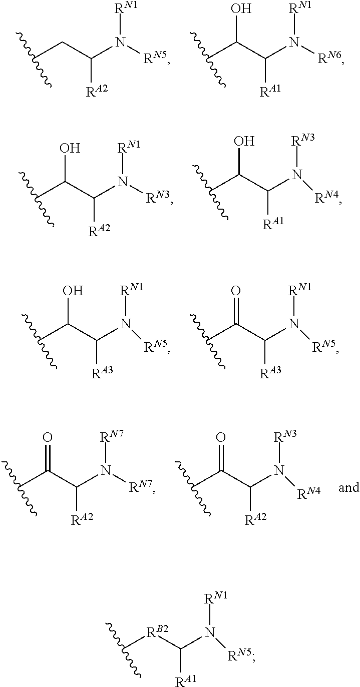

R^{5A}, R^{6A}, R^{7A}, and R^{8A} are independently selected from R¹ and

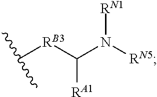

wherein 5 of the 6 of R^{2A}, R^{3A}, R^{5A}, R^{6A}, R^{7A}, and R^{8A} are R¹; and all other variables are as defined herein.

In certain embodiments the compound of Formula II is provided as a pure R- or S-enantiomer or enantiomerically enriched mixture.

In certain embodiments the compound of Formula II is of Formula IIa:

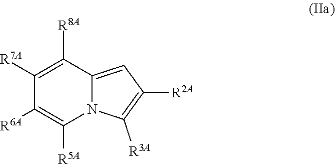

or a pharmaceutically acceptable salt or salt mixture thereof;
wherein:
for compounds of Formula IIa every R¹ is hydrogen.

The R- or S-enantiomers of the present invention can exist in isolated form or mixed in such a way that one enantiomer is present in a greater amount than the other, referred to herein as an enantiomerically enriched mixture. An enantiomerically enriched mixture is a mixture that contains one enantiomer in a greater amount than the other. The term enantiomerically enriched mixture includes either the mixture enriched with the R-enantiomer or enriched with the S-enantiomer. Unless context clearly indicates otherwise, the term “enantiomerically enriched mixture” can be understood to mean “enantiomerically enriched mixture of the R- or S-enantiomer.” An enantiomerically enriched mixture of an S-enantiomer contains at least 55% of the S-enantiomer, and, typically at least about 60%, 65%, 70%, 75%, 80%, 85%, 90%, 95% of the S-enantiomer. An enantiomerically enriched mixture of an R-enantiomer contains at least 55% of the R-enantiomer, and typically at least about 60%, 65%, 70%, 75%, 80%, 85%, 90%, 95% of the R-enantiomer. The specific ratio of S or R enantiomer can be selected for the need of the patient according to the health care specialist to balance the desired effect. Typically, in the present application, the chiral carbon referred to in the term “enantiomerically enriched” is that carbon alpha to the amine in the provided structures.

The term enantiomerically enriched mixture as used herein typically does not include either a racemic mixture or a pure enantiomer.

For example, in some embodiments of the invention, compounds of Formula II are provided as pure R- or S-enantiomers or enriched in the R- or S-enantiomeric form, as described by Formulas IIb and IIc:

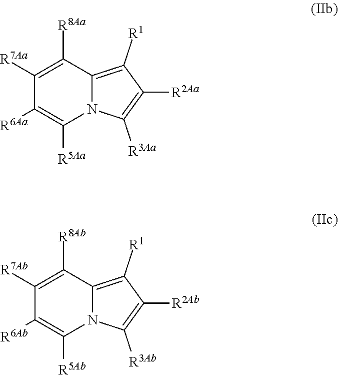

or a pharmaceutically acceptable salt or salt mixture thereof;
wherein:
R^{2A} is selected from R¹,

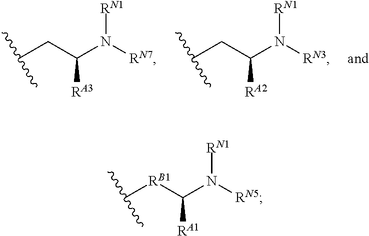

R^{2Ab} is selected from R¹,

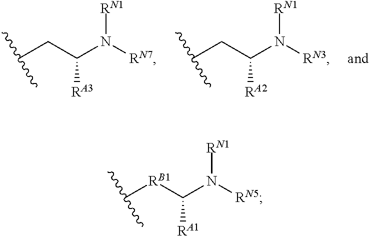

R^{3Aa} is selected from R¹,

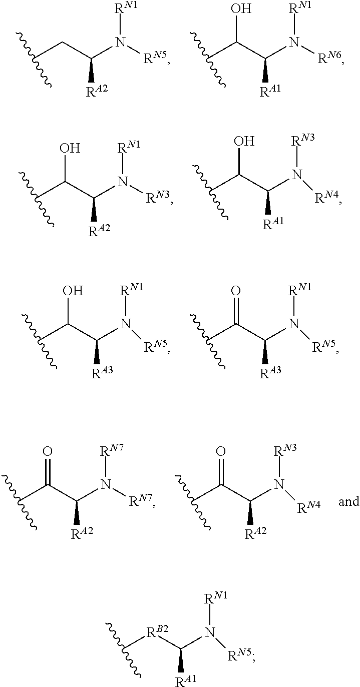

R^{3Ab} is selected from R¹,

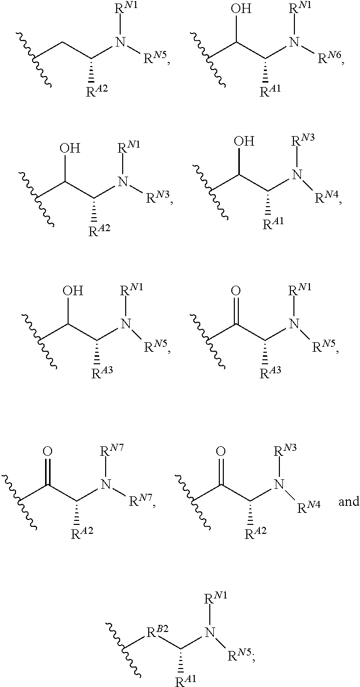

R^{5Aa}, R^{6Aa}, R^{7Aa}, and R^{8Aa} are independently selected from R¹ and

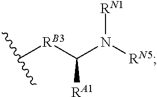

R^{5Ab}, R^{6Ab}, R^{7Ab}, and R^{8Ab} are independently selected from R¹ and

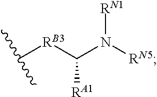

and 5 of the 6 of R^{2Aa}, R^{3Aa}, R^{5Aa}, R^{6Aa}, R^{7Aa}, and R^{8Aa} are not R¹;
and 5 of the 6 of R^{2Ab}, R^{3Ab}, R^{5Ab}, R^{6Ab}, R^{7Ab}, and R^{8Ab} are R¹; and all other variables are as defined herein.

Therefore, in some embodiments an enantiomerically enriched mixture of a compound of Formula I or II is carefully tuned to achieve desired results for the patient by altering the ratio of enantiomers to maximize serotonin-receptor-dependent therapeutic effects and minimize unwanted effects.

In another aspect of the invention, a compound of Formula III is provided as an enantiomerically enriched mixture:

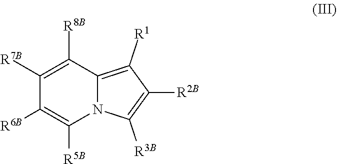

or a pharmaceutically acceptable salt or salt mixture thereof;
wherein:
R^{2B}, R^{3B}, R^{5B}, R^{6B}, R^{7B}, and R^{8B} are independently selected from R¹ and

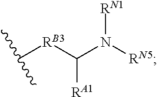

wherein 5 of the 6 of R^{2B}, R^{3B}, R^{5B}, R^{6B}, R^{7B}, and R^{8B} are R¹; and all other variables are as defined herein.

In certain embodiments the compound of Formula III is of Formula IIIa:

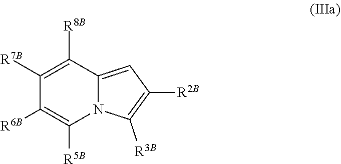

or a pharmaceutically acceptable salt or salt mixture thereof;
wherein:
for compounds of Formula IIIa every R¹ is hydrogen.

The compounds described herein can be administered in an effective amount to treat any of the mental disorders described herein or to provide mental enhancement to a human patient in need of thereof. In certain embodiments a compound described herein can be used to treat a host such as a human in need thereof as a milder therapeutic than MDMA and which is faster acting than typical SSRIs. This enhances the patient experience and encourages the needed medical therapy. In certain embodiments a compound described herein increases empathy, sympathy, openness and/or acceptance of oneself and others. This compound can be taken, if necessary, as part of one or more therapeutic counseling sessions, or when necessary, episodically, or even consistently, as prescribed by a healthcare provider. In some embodiments, a compound of the present invention acts within a reasonable waiting time in a clinic and lasts for one, two, or several hours or otherwise in a time sufficient to complete the therapy session and then diminishes in effect sufficiently for the patient to leave the clinic and resume normal activities. In other embodiments, the compound of the present invention is administered in a periodic or consistent dosage, including a daily dosage in a similar manner to an anti-depressant drug, to enhance self-acceptance, acceptance of others and a general feeling of peace and comfort with surroundings and events.

The present invention includes in certain embodiments a compound with beneficial selectivity profiles for neurotransmitter transporters. In certain embodiments, a compound of the present invention provides a dopamine transporter (DAT) to serotonin transporter (SERT) ratio of less than one.

In some embodiments, the indolizine compound of the current invention, as a racemic mixture, enantiomerically enriched mixture or pure enantiomer is a direct 5-HT_{1B} agonist. In yet further embodiments, an indolizine compound of the current invention is also a 5-HT releaser.

In some embodiments, the indolizine compound of the current invention, as a racemic mixture, enantiomerically enriched mixture or pure enantiomer has a duration of acute therapeutic effects that is less than that of MDMA (reported to be 4.2 hours with a standard deviation of 1.3 hours after 75 or 125 mg MDMA by Vizeli & Liechti. 2017. Journal of Psychopharmacology, 31(5), 576-588). This can be desirable for reducing the costs and resources needed for pharmacotherapy sessions. In other embodiments, the indolizine compound of the current invention, as a racemic mixture, enantiomerically enriched mixture or pure enantiomer has a duration of acute therapeutic effects that is greater than that of MDMA. This avoids the need for re-administration of the entactogen, which produces nonlinear increases in plasma concentrations and greater unwanted effects.

In some embodiments, the indolizine compound of the current invention, as a racemic mixture, enantiomerically enriched mixture or pure enantiomer produces acute cardiovascular effects that are less than those of MDMA. MDMA produces acute tachycardia and hypertension, which requires safety monitoring and may limit its use in those with preexisting cardiovascular disease (Vizeli & Liechti. 2017. Journal of Psychopharmacology, 31(5), 576-588; MDMA Investigator's Brochure, 13th Edition: Mar. 22, 2021).

In some embodiments a compound of the present invention has favorable pharmacokinetic properties for administration to a mammal, for example a human. These properties can include having more reproducible and less variable pharmacokinetic properties than MDMA. In certain embodiments, a compound of the present invention has a less variable maximum plasma concentration (Cₘₐₓ) than MDMA. In certain embodiments, a compound of the present invention has a less variable area-under-the-concentration-versus-time-curve (AUC) than MDMA. An additional potential beneficial property of a compound of the present invention is reduced inhibition of CYP enzymes compared to MDMA. Inhibition of such enzymes can cause unwanted toxic drug-drug interactions. In certain embodiments, a compound of the present invention does not inhibit or shows minimal inhibition of cytochrome p450 isozyme 2D6 (CYP2D6). In certain embodiments, a compound of the present invention shows less potent inhibition of CYP2D6 than MDMA.

In further embodiments, an indolizine compound of the current invention is a direct 5-HT_{2A} agonist. In yet further embodiments, an indolizine compound of the current invention is a 5-HT releaser. 5-HT_{2A} agonists increase neuroplasticity and decrease inflammation and are currently being investigated for a variety of indications, including for treating chronic pain, headache, depression, anxiety, and substance use disorders. Most substances that are 5-HT_{2A} agonists have significant side effects that are often undesirable in a therapeutic context. For example, psilocybin often produces labile mood with frequent anxiety, derealization, and depersonalization, which are signs and symptoms that limit clinical use. In some aspects of the present invention, an indolizine compound releases 5-HT and is a 5-HT_{2A} agonist while displaying greatly decreased side effects compared to psilocybin, LSD, and other clinically used 5-HT_{2A} agonists.

In typical embodiments, pharmaceutical compositions are disclosed which comprise a compound of any of Structures I through XLV, either racemic, as pure enantiomers, or in an enantiomerically enriched mixture, and which may be in association with another active agent, as well as with a pharmaceutically acceptable carrier, diluent, or excipient.

In some embodiments, an enantiomerically enriched mixture of the S-enantiomer or pure enantiomer of Formula I or II increases the serotonin-receptor-dependent actions that contribute to therapeutic effects and minimizes adverse dopaminergic effects that can contribute to unwanted properties like addictive liability when administered to a host in need thereof, for example a mammal, including a human, relative to the racemic form.

In some embodiments, an enantiomerically enriched mixture of the R-enantiomer or pure enantiomer of Formula I or II increases the serotonin-receptor-dependent actions that contribute to therapeutic effects and minimizes adverse dopaminergic effects that can contribute to unwanted properties like addictive liability when administered to a host in need thereof, for example a mammal, including a human, relative to the racemic form.

In further embodiments, pharmaceutical compositions are disclosed which comprise a compound of Formula I, II, or III, either racemic, as pure enantiomers, or in an enantiomerically enriched mixture, and which may be in association with another active agent, in a pharmaceutically acceptable composition that has a carrier, diluent, or excipient. The pharmaceutical compositions of the present invention may in certain embodiments include a salt mixture, wherein a salt mixture may comprise 1, 2 or more different pharmaceutically acceptable salts together to form a single composition. In some embodiments, enantiomers are mixed that each has a different salt or wherein there is a ratio of salts, as in Adderall, for example, which is a mixture of a racemate of amphetamine as an aspartate salt, racemate of amphetamine as a sulfate salt, and D-amphetamine as a saccharate salt and D-amphetamine as a sulfate salt. These kinds of mixtures of racemic, enantiomerically enriched and pure compounds of Formulas I, II, or III can provide advantageous results.

The invention includes methods for modulating the activity of the CNS of a host in need thereof, such as a human, by administering an effective amount of a compound or composition of the invention. Examples are methods for treating a variety of CNS disorders, as generally listed herein, that have been linked to inadequate functioning of serotonergic neurotransmission in mammals, typically a human, using a compound or composition of the invention. The invention also includes methods of improving CNS functioning such as reducing neuroticism or psychological defensiveness or increasing creativity, decision-making ability, or openness to experience in a human by administering an effective amount of a compound or composition of the invention.

Specifically, the invention includes methods to treat a neurological or psychiatric central nervous system disorder as further described herein, including a mental disorder, or to provide a mental enhancement, with a compound of Structures I-XLV, Formula I, II, or III, or a pharmaceutically acceptable salt or salt mixture thereof.

Additionally, the invention includes a method of treating a patient with primary or secondary headaches is provided, comprising administering an effective amount of a compound, pure enantiomer, or enantiomerically enriched mixture of Formula I, II, or III.

These and other objects, features, and advantages of the present invention may be more clearly understood and appreciated from a review of the following detailed description of the disclosed embodiments and examples, and by reference to the appended claims.

The present invention thus includes at least the following aspects:
(i) A compound of Structures I-XLV, Formula I, Formula II, or a pharmaceutically acceptable salt or salt mixture, isotopic derivative, or prodrug thereof;
(ii) An enantiomerically enriched or pure enantiomer of Structures I-XLV, Formula I, or Formula II, or a pharmaceutically acceptable salt or salt mixture, isotopic derivative, or prodrug thereof;
(iii) An enantiomerically enriched mixture of Structures I-XLV, Formula I, Formula II, or Formula III, or a pharmaceutically acceptable salt or salt mixture, isotopic derivative, or prodrug thereof;
(iv) A pharmaceutical composition comprising an effective patient-treating amount of a compound of (i), (ii) or (iii) in a pharmaceutically acceptable carrier or diluent for any of the uses described herein;
(v) The pharmaceutically acceptable composition of (iv) in a solid or liquid, systemic, oral, topical or parenteral dosage form;
(vi) A method for treating a patient with any neurological or psychological CNS disorder as described herein that includes administering an effective amount of a compound of (i), (ii) or (iii) to a patient such as a human in need thereof,
(vii) A method for treating any neurological or psychological CNS disorder comprising administering an effective amount of a compound of (i), (ii) or (iii) or a pharmaceutically acceptable salt, isotopic derivative, or prodrug thereof, as described herein, to a patient, typically a human, in need thereof;
(viii) A compound of (i), (ii) or (iii) or a pharmaceutically acceptable salt, salt mixture, isotopic derivative, or prodrug thereof, for use to treat any disorder as described herein in an effective amount as further described herein;
(ix) A compound of (i), (ii) or (iii) for use in the manufacture of a medicament for the treatment of any of the disorders described herein;
(x) Use of a compound of (i), (ii) or (iii) or a pharmaceutically acceptable salt, salt mixture, isotopic derivative, or prodrug thereof, to treat any disorder as described herein in an effective amount as further described herein;
(xi) Processes for the preparation of therapeutic products that contain an effective amount of a compound of (i), (ii) or (iii) or a pharmaceutically acceptable salt or salt mixtures, isotopic derivatives, or prodrugs or compositions thereof, as described herein.

### BRIEF DESCRIPTION OF THE FIGURES

FIG. 1 depicts response curves for 1-(indolizin-3-yl)-2-(methylamino)propan-1-one (Structure XLX) in a serotonin (SERT) release assay and a dopamine (DAT) release assay. The x-axis is concentration measured in molar wherein the notation 1e-X refers to log base 10. The y-axis is normalized response measured in %. The experimental procedure is provided in Example 12.

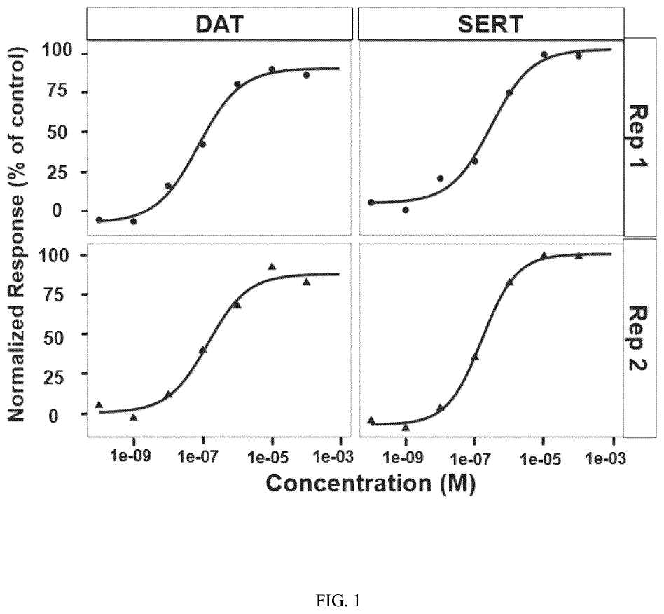

FIG. 2 is a response curve for 1-(indolizin-3-yl)-2-(methylamino)propan-1-one (Structure XLX) in a HTR1B agonist activity assay. The experimental procedure is provided in Example 11. The x-axis is concentration measured in micromolar. The y-axis is normalized response measured in %.

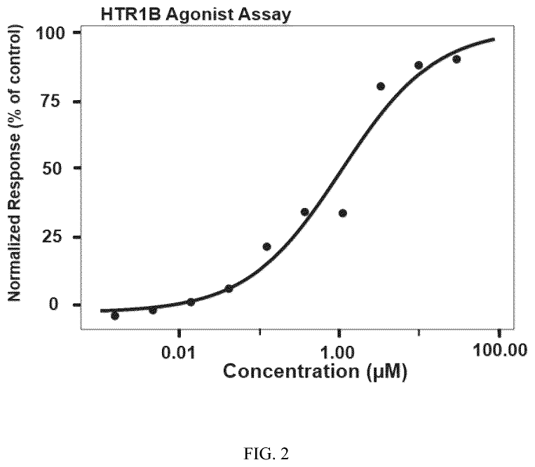

FIG. 3 is a response curve for 1-(indolizin-3-yl)-2-(methylamino)propan-1-one (Structure XLX) in a HTR2B antagonist activity assay. The x-axis is concentration measured in micromolar. The y-axis is normalized response measured in %. The experimental procedure is provided in Example 11.

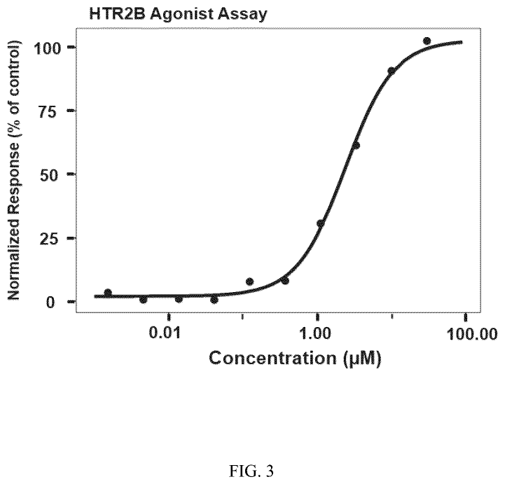

FIG. 4 is a response curve for 1-(indolizin-3-yl)-2-(methylamino)propan-1-one (Structure XLX) in a norepinephrine transporter (NET) blocker assay. The x-axis is concentration measured in micromolar. The y-axis is normalized response measured in %. The experimental procedure is provided in Example 11.

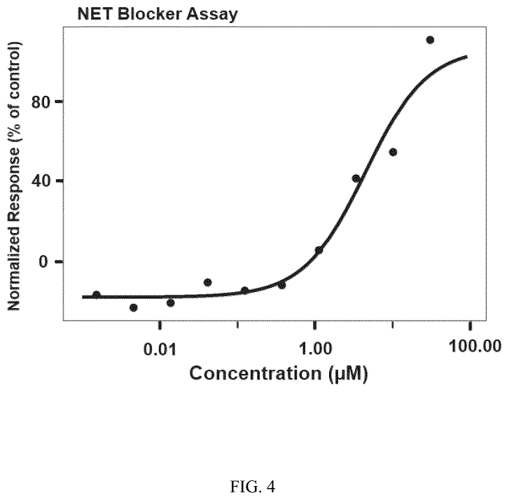

FIG. 5 is a response curve for 1-(indolizin-3-yl)-2-(methylamino)propan-1-one (Structure XLX) in a dopamine transporter blocker assay. The x-axis is concentration measured in micromolar. The y-axis is normalized response measured in %. The experimental procedure is provided in Example 11.

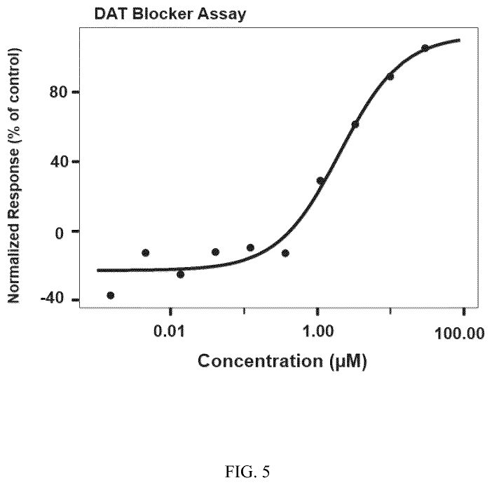

FIG. 6 is a response curve for 1-(indolizin-3-yl)-2-(methylamino)propan-1-one (Structure XLX) in a serotonin transporter blocker assay. The x-axis is concentration measured in micromolar. The y-axis is normalized response measured in %. The experimental procedure is provided in Example 11.

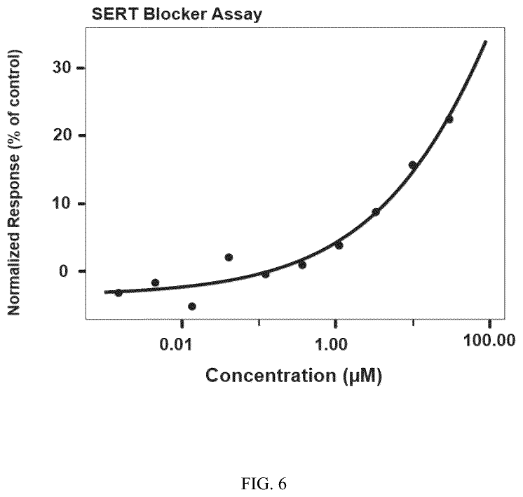

FIG. 7 depicts Formula I of the present invention.

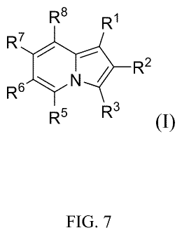

### DETAILED DESCRIPTION OF THE INVENTION

The present invention includes indolizines of Structures I-XLV, Formula I, II, or III, or a pharmaceutically acceptable salt or salt mixture, isotopic derivative, or prodrug or pharmaceutically acceptable composition thereof, as well as methods for modulation of CNS activity, and for treatment of CNS disorders, including but not limited to post-traumatic stress, depression, adjustment disorders, addiction, anxiety and other mental disorders as described herein to a host such as a human in need thereof. The indolizines of the present invention provide advantageous pharmacological properties that are desirable as therapeutics for the treatment of mental disorders, particularly as psychotherapeutics and neurotherapeutics.

In certain embodiments a compound described herein is a milder therapeutic than the entactogen MDMA and is faster acting than common SSRIs. In addition, the indolizines of Structures I-XLV or Formula I, II, or III may induce fewer unwanted side effects caused by dopaminergic or adrenaline agonism which decreases the patient experience, are counterproductive to the therapy, and/or are undesirably toxic. In certain embodiments a compound described herein increases empathy, sympathy, openness and/or acceptance of oneself and others. This compound can be taken, if necessary, as part of therapeutic counseling sessions, or when necessary, episodically, or even consistently, as prescribed by a healthcare provider.

WO 2006/050007 filed by Wyeth describes certain indolizines for the treatment of CNS disorders. US20140162939 discloses the use of indolizines as antibacterial agents. WO2008029152 describes the use of certain aminobutan-1-one-substituted indolizines for the treatment of Duchenne muscular dystrophy. WO2019118909 discloses some as inhibitors of SH2 containing protein tyrosine phosphatase-2 (SHP2), which is a phosphatase of interest in oncology. WO2008134553 discloses methods of treating sodium channel-mediated diseases with bicyclic compounds, including indolizine derivatives. JP1995242666 discloses use of indolizine derivatives as bradykinin antagonists.

U.S. Pat. Nos. 6,069,282 and 6,169,213 disclose methods of synthesis that can be used to produce certain indolizines. DE19723961, EP1129690, EP1129691, and DE19723890 describe the use of certain indolizines in dyeing.

### Definitions

When introducing elements of the present invention or the typical embodiments thereof, the articles “a,” “an,” “the,” and “said” are intended to mean that there are one or more of the elements. The terms “comprising,” “including,” and “having” are intended to be inclusive and not exclusive (i.e., there may be other elements in addition to the recited elements). Thus, the terms “including,” “may include,” and “include,” as used herein mean, and are used interchangeably with, the phrase “including but not limited to.”

Where a range of values is provided, it is understood that the upper and lower limit, and each intervening value between the upper and lower limit of the range is encompassed within the embodiments.

Unless defined otherwise, all technical and scientific terms herein have the meaning as commonly understood by one of ordinary skill in the art to which this invention belongs. In the event there is a plurality of definitions for a term herein, those in this section prevail unless stated otherwise. Further definitions that may assist the reader to understand the disclosed embodiments are as follows, and such definitions may be used to interpret the defined terms, when those terms are used herein. However, the examples given in the definitions are generally non-exhaustive and must not be construed as limiting the invention. It also will be understood that a substituent should comply with chemical bonding rules and steric compatibility constraints in relation to the particular molecule to which it is attached.

A compound of the invention may contain one or more chiral centers and/or double bonds and therefore, may exist as stereoisomers, such as double-bond isomers (i.e., geometric isomers), enantiomers, or diastereomers. Unless otherwise indicated the chemical structures depicted herein independently encompass all possible enantiomers and stereoisomers of the illustrated compounds including the stereoisomerically pure form (for example, geometrically pure, enantiomerically pure, or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan.

An enantiomerically enriched mixture is a mixture that contains one enantiomer in a greater amount than the other. An enantiomerically enriched mixture of an S-enantiomer contains at least 55% of the S-enantiomer, and, typically at least about 60%, 65%, 70%, 75%, 80%, 85%, 90%, or 95% or more of the S-enantiomer and typically not more than 98%. An enantiomerically enriched mixture of an R-enantiomer contains at least 55% of the R-enantiomer, and typically at least about 60%, 65%, 70%, 75%, 80%, 85%, 90% or 95% of the R-enantiomer and typically not more than 98%. The specific ratio of S or R enantiomer can be selected for the need of the patient according to the health care specialist to balance the desired effect. In certain embodiments, as indicated by context, the term enantiomerically enriched does not include a pure enantiomer.

The term enantiomerically enriched mixture as used in this application does not include a racemic mixture and does not include a pure isomer. Notwithstanding, it should be understood that any compound described herein in enantiomerically enriched form can be used as a pure isomer if it achieves the goal of any of the specifically itemized methods of treatment described herein, including but not limited Structures I-XLV, or a compound, pure enantiomer or enantiomerically enriched mixture of Formula I, II, or III.

In certain embodiments the term enantiomerically pure as used herein refers to a compound with about 98% to 100% stereochemical purity.

The term “CNS disorder” as used herein refers to either a neurological condition (one that is typically treated by a neurologist) or a psychiatric condition (one that is typically treated by a psychiatrist). Neurological disorders are typically those affecting the structure, biochemistry or normal electrical functioning of the brain, spinal cord or other nerves. Psychiatric conditions are more typically thought of as mental disorders, which are primarily abnormalities of thought, feeling or behavior that cause significant distress or impairment of personal functioning. Thus, a disclosed compound can be used in an effective amount to improve neurological or psychiatric functioning in a patient in need thereof. Neurological indications include, but are not limited to improved neuroplasticity, including treatment of stroke, brain trauma, dementia, and neurodegenerative diseases. A compound of the current invention can be considered a psychoplastogen, that is, a small molecule that is able to induce rapid neuroplasticity. For example, in certain embodiments, the disclosed compound or composition can be used to improve stuttering and other dyspraxias or to treat Parkinson's disease or schizophrenia.

The term “neurological disease or disorder” includes Alzheimer's disease, mild cognitive impairment (MCI), Parkinson's disease, Parkinson's disease dementia, multiple sclerosis, adrenoleukodystrophy, AIDS dementia complex, Alexander disease, Alper's disease, amyotrophic lateral sclerosis (ALS), ataxia telangiectasia, Batten disease, bovine spongiform encephalopathy, Canavan disease, cerebral amyloid angiopathy, cerebellar ataxia, Cockayne syndrome, corticobasal degeneration, Creutzfeldt-Jakob disease, diffuse myelinoclastic sclerosis, fatal familial insomnia, Fazio-Londe disease, Friedreich's ataxia, frontotemporal dementia or lobar degeneration, hereditary spastic paraplegia, Huntington disease, Kennedy's disease, Krabbe disease, Lewy body dementia, Lyme disease, Machado-Joseph disease, motor neuron disease, Multiple systems atrophy, neuroacanthocytosis, Niemann-Pick disease, Pelizaeus-Merzbacher Disease, Pick's disease, primary lateral sclerosis including its juvenile form, progressive bulbar palsy, progressive supranuclear palsy, Refsum's disease including its infantile form, Sandhoff disease, Schilder's disease, spinal muscular atrophy, spinocerebellar ataxia, Steele-Richardson-Olszewski disease, subacute combined degeneration of the spinal cord, survival motor neuron spinal muscular atrophy, Tabes dorsalis, Tay-Sachs disease, toxic encephalopathy, transmissible spongiform encephalopathy, Vascular dementia, X-linked spinal muscular atrophy, synucleinopathy, progranulinopathy, tauopathy, amyloid disease, prion disease, protein aggregation disease, and movement disorder.

The term “improving psychiatric function” is intended to include mental health and life conditions that are not traditionally treated by neurologists but sometimes treated by psychiatrists and can also be treated by psychotherapists, life coaches, personal fitness trainers, meditation teachers, counselors, and the like. For example, it is contemplated that a disclosed compound will allow individuals to effectively contemplate actual or possible experiences that would normally be upsetting or even overwhelming. This includes individuals with fatal illness planning their last days and the disposition of their estate. This also includes couples discussing difficulties in their relationship and how to address them. This also includes individuals who wish to more effectively plan their career.

The term “inadequate functioning of neurotransmission” is used synonymously with a CNS disorder that adversely affects normal healthy neurotransmission.

Examples of isotopes that can be incorporated into a compound of the invention include isotopes of hydrogen, carbon, nitrogen, oxygen, fluorine and chlorine such as ²H, ³H, ¹¹C, ¹³C, ¹⁴C, ¹³N ¹⁵N, ¹⁷O, ¹⁸O, ¹⁸F, ³⁶Cl, and respectively. In some non-limiting embodiments, an isotopically labelled compound can be used in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F labeled compound may be particularly desirable for PET or SPECT studies. An isotopically labeled compound of this invention and a prodrug thereof can generally be prepared by carrying out the procedures disclosed in the schemes or in the examples and preparations described below by substituting a readily available isotopically labeled reagent for a non-isotopically labeled reagent.

By way of general example and without limitation, isotopes of hydrogen, for example, deuterium (²H) and tritium (³H) may be used anywhere in described structures that achieves the desired result. Alternatively, or in addition, isotopes of carbon, for example, ¹³C and ¹⁴C, may be used.

Isotopic substitutions, for example deuterium substitutions, can be partial or complete. Partial deuterium substitution means that at least one hydrogen is substituted with deuterium. In certain embodiments, the isotope is at least 60, 70, 80, 90, 95 or 99% or more enriched in an isotope at any location of interest. In some non-limiting embodiments, deuterium is at least 80, 90, 95 or 99% enriched at a desired location. Unless indicated to the contrary, the deuteration is at least 80% at the selected location. Deuteration can occur at any replaceable hydrogen that provides the desired results.

In some non-limiting embodiments, the substitution of a hydrogen atom for a deuterium atom can be provided in a compound or composition described herein. For example, when any of the groups are, or contain for example through substitution, methyl, ethyl, or methoxy, the alkyl residue may be deuterated (in non-limiting embodiments, CDH₂, CD₂H, CD₃, CH₂CD₃, CD₂CD₃, CHDCH₂D, CH₂CD₃, CHDCHD₂, OCDH₂, OCD₂H, or OCD₃ etc.). A compound of the invention also includes an isotopically labeled compound where one or more atoms have an atomic mass different from the atomic mass conventionally found in nature. Examples of isotopes that may be incorporated into a compound of the invention include ²H, ³H, ¹³C, ¹⁴C, ¹³N, ¹⁵N, ¹⁸O, ¹⁷O, ³¹P, ³²P, ³⁵S, ¹⁸F, and ³⁶Cl.

An alkyl group on the nitrogen of Formula I, II, or III of the invention is subject to enzymatic removal. The N-alkyl may be prepared with a deuterated reagent that replaces one, two, any, or all of the hydrogens on the N-alkyl group, which creates a higher activation energy for bond cleavage and a slower formation of the desalkyl metabolite. In general, when deuterium is substituted for a hydrogen at a location of metabolism in the compound, a more stable compound will result.

Any one of Structures I-XLV or a compound, pure enantiomer or enantiomerically enriched mixture of Formula I, II, or III of the invention has a chiral center and thus exists as enantiomers that may be more appropriate for some applications. Accordingly, the present disclosure also includes stereoisomers of a compound described herein, where applicable, either individually or admixed in any proportions. Stereoisomers may include enantiomers, diastereomers, racemic mixtures, and combinations thereof.

“Stereoisomers” includes enantiomers, diastereomers, the components of racemic mixtures, and combinations thereof. Stereoisomers can be prepared or separated as described herein or by using other methods.

Such stereoisomers can be prepared and separated using conventional techniques, either by reacting enantiomeric starting materials, or by separating isomers of a compound disclosed herein.

“Agonist” refers to a modulator that binds to a receptor or enzyme and activates the receptor to produce a biological response. In some embodiments, “agonist” includes full agonists or partial agonists.

“Antagonism” refers to the inactivation of a receptor or enzyme by a modulator, or antagonist. Antagonism of a receptor, for example, is when a molecule binds to the receptor and does not allow activity to occur or reduces activity.

“IC₅₀” refers to the concentration of a substance (for example, a compound or a drug) that is required for 50% inhibition of a biological process. For example, IC₅₀ refers to the half maximal (50%) inhibitory concentration (IC) of a substance as determined in a suitable assay. Similarly, EC₅₀ refers to the concentration of a substance that provokes a response halfway between the baseline activity and maximum response. In some instances, an IC₅₀ or EC₅₀ is determined in an in vitro assay system. In some embodiments as used herein, IC₅₀ (or EC₅₀) refers to the concentration of a modulator that is required for 50% inhibition (or excitation) of a receptor, for example, 5HT_{1B}.

“Modulate” or “modulating” or “modulation” refers to an increase or decrease in the amount, quality, or effect of a particular activity, function or molecule. By way of illustration and not limitation, agonists, partial agonists, antagonists, and allosteric modulators (for example, positive allosteric modulator) of a G protein-coupled receptor (for example, 5-HT_{1B}) are modulators of the receptor.

“Neuroplasticity” refers to the ability of the brain to change its structure and/or function throughout a subject's life. Examples of the changes to the brain include, but are not limited to, the ability to adapt or respond to internal and/or external stimuli, such as due to an injury, and the ability to produce new neurites, dendritic spines, and synapses.

“Treating” or “treatment” of a disease, as used in context, includes (i) inhibiting the disease, i.e., arresting or reducing the development or progression of the disease or its clinical symptoms; or (ii) relieving the disease, i.e., causing regression of the disease or its clinical symptoms. Inhibiting the disease, for example, would include prophylaxis. Hence, one of skill in the art will understand that a therapeutic amount necessary to effect treatment for purposes of this invention will, for example, be an amount that provides for objective indicia of improvement in patients having clinically diagnosable symptoms. Other such measurements, benefits, and surrogate or clinical endpoints, whether alone or in combination, would be understood to those of ordinary skill.

“Therapeutic effect” means the responses(s) in a host after treatment that is judged to be desirable or beneficial. Hence, depending on the CNS disorder to be treated, or improvement in CNS functioning sought, those responses shall differ, but would be readily understood by those of ordinary skill.

### Indolizines of the Present Invention

The present invention includes but is not limited to compounds, pure enantiomers, and enantiomerically enriched mixtures of the structures shown in Table 1. These compounds, pure enantiomers, or enantiomerically enriched mixtures are optionally provided as the pharmaceutically acceptable salt or as a salt mixture thereof.

TABLE 1
Exemplary Compounds and Pure Enantiomers of the Present Invention

| Compound Structure | Compound Name |
|---|---|
| 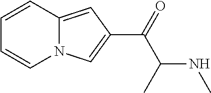 | Structure I |
| S-enantiomer of Structure I | Structure Ia |
| R-enantiomer of Structure I | Structure Ib |
| 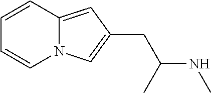 | Structure II |
| S-enantiomer of Structure II | Structure IIa |
| R-enantiomer of Structure II | Structure IIb |
| 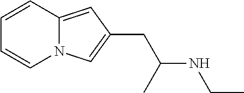 | Structure III |
| S-enantiomer of Structure III | Structure IIIa |
| R-enantiomer of Structure III | Structure IIIb |
| 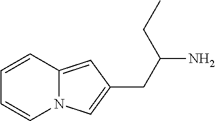 | Structure IV |
| S-enantiomer of Structure IV | Structure IVa |
| R-enantiomer of Structure IV | Structure IVb |
| 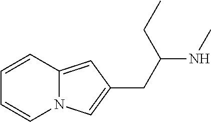 | Structure V |
| S-enantiomer of Structure V | Structure Va |
| R-enantiomer of Structure V | Structure Vb |
| 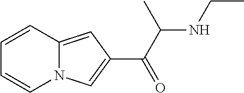 [SMILES: CCNC(C)C(=O)c1cc2ccccn2c1] | Structure VI |
| S-enantiomer of Structure VI | Structure VIa |
| R-enantiomer of Structure VI | Structure VIb |
| 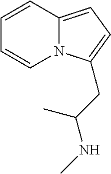 | Structure VII |
| S-enantiomer of Structure VII | Structure VIIa |
| R-enantiomer of Structure VII | Structure VIIb |
| 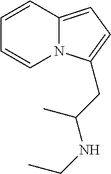 | Structure VIII |
| S-enantiomer of Structure VIII | Structure VIIIa |
| R-enantiomer of Structure VIII | Structure VIIIb |
| 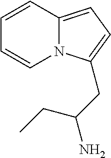 | Structure IX |
| S-enantiomer of Structure IX | Structure IXa |
| R-enantiomer of Structure IX | Structure IXb |
| 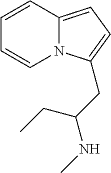 | Structure X |
| S-enantiomer of Structure X | Structure Xa |
| R-enantiomer of Structure X | Structure Xb |
| 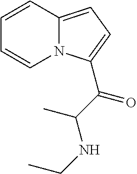 [SMILES: CCNC(C)C(=O)c1ccc2ccccn12] | Structure XI |
| S-enantiomer of Structure XI | Structure XIa |
| R-enantiomer of Structure XI | Structure XIb |
| 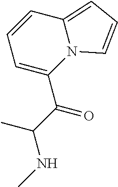 | Structure XII |
| S-enantiomer of Structure XII | Structure XIIa |
| R-enantiomer of Structure XII | Structure XIIb |
| 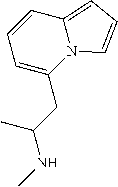 | Structure XIII |
| S-enantiomer of Structure XIII | Structure XIIIa |
| R-enantiomer of Structure XIII | Structure XIIIb |
| 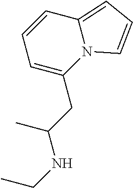 | Structure XIV |
| S-enantiomer of Structure XIV | Structure XIVa |
| R-enantiomer of Structure XIV | Structure XIVb |
| 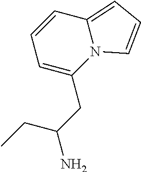 | Structure XV |
| S-enantiomer of Structure XV | Structure XVa |
| R-enantiomer of Structure XV | Structure XVb |
| 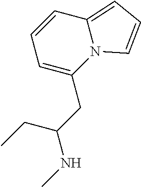 | Structure XVI |
| S-enantiomer of Structure XVI | Structure XVIa |
| R-enantiomer of Structure XVI | Structure XVIb |
| 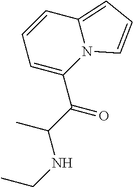 [SMILES: CCNC(C)C(=O)c1cccc2cccn12] | Structure XVII |
| S-enantiomer of Structure XVII | Structure XVIIa |
| R-enantiomer of Structure XVII | Structure XVIIb |
| 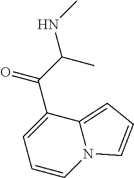 | Structure XVIII |
| S-enantiomer of Structure XVIII | Structure XIIIa |
| R-enantiomer of Structure XVIII | Structure XIIIb |
| 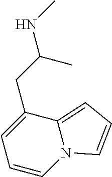 | Structure XIX |
| S-enantiomer of Structure XIX | Structure XIXa |
| R-enantiomer of Structure XIX | Structure XIXb |
| 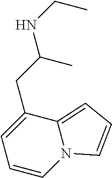 | Structure XX |
| S-enantiomer of Structure XX | Structure XXa |
| R-enantiomer of Structure XX | Structure XXb |
| 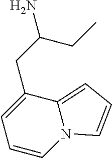 | Structure XXI |
| S-enantiomer of Structure XXI | Structure XXIa |
| R-enantiomer of Structure XXI | Structure XXIb |
| 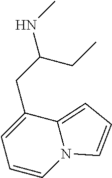 | Structure XXII |
| S-enantiomer of Structure XXII | Structure XXIIa |
| R-enantiomer of Structure XXII | Structure XXIIb |
| 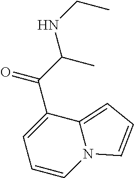 [SMILES: CCNC(C)C(=O)c1cccn2cccc12] | Structure XXIII |
| S-enantiomer of Structure XXIII | Structure XXIIIa |
| R-enantiomer of Structure XXIII | Structure XXIIIb |
| 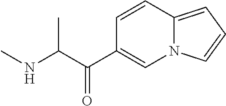 | Structure XXIV |
| S-enantiomer of Structure XXIV | Structure XXIVa |
| R-enantiomer of Structure XXIV | Structure XXIVb |
| 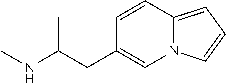 | Structure XXV |
| S-enantiomer of Structure XXV | Structure XXVa |
| R-enantiomer of Structure XXV | Structure XXVb |
| 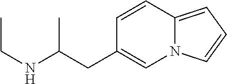 | Structure XXVI |
| S-enantiomer of Structure XXVI | Structure XXVIa |
| R-enantiomer of Structure XXVI | Structure XXVIb |
| 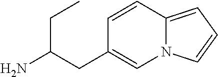 | Structure XXVII |
| S-enantiomer of Structure XXVII | Structure XXVIIa |
| R-enantiomer of Structure XXVII | Structure XXVIIb |
| 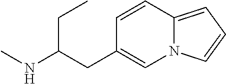 | Structure XXVIII |
| S-enantiomer of Structure XXVIII | Structure XXVIIIa |
| R-enantiomer of Structure XXVIII | Structure XXVIIIb |
| 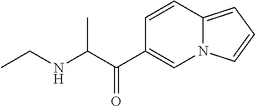 [SMILES: CCNC(C)C(=O)c1ccc2cccn2c1] | Structure XXIX |
| S-enantiomer of Structure XXIX | Structure XXIXa |
| R-enantiomer of Structure XXIX | Structure XXIXb |
| 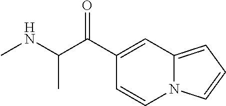 | Structure XXX |
| S-enantiomer of Structure XXX | Structure XXXa |
| R-enantiomer of Structure XXX | Structure XXXb |
| 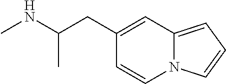 | Structure XXXI |
| S-enantiomer of Structure XXXI | Structure XXXIa |
| R-enantiomer of Structure XXXI | Structure XXXIb |
| 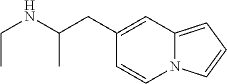 | Structure XXXII |
| S-enantiomer of Structure XXXII | Structure XXXIIa |
| R-enantiomer of Structure XXXII | Structure XXXIIb |
| 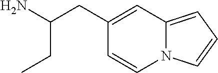 | Structure XXXIII |
| S-enantiomer of Structure XXXIII | Structure XXXIIIa |
| R-enantiomer of Structure XXXIII | Structure XXXIIIb |
| 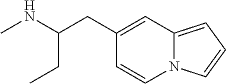 | Structure XXXIV |
| S-enantiomer of Structure XXXIV | Structure XXXIVa |
| R-enantiomer of Structure XXXIV | Structure XXXIVb |
| 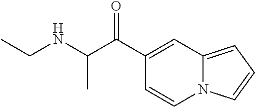 [SMILES: CCNC(C)C(=O)c1ccn2cccc2c1] | Structure XXXV |
| S-enantiomer of Structure XXXV | Structure XXXVa |
| R-enantiomer of Structure XXXV | Structure XXXVb |
| 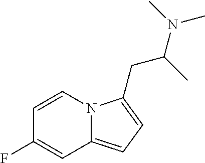 [SMILES: CC(Cc1ccc2cc(F)ccn12)N(C)C] | Structure XXXVI |
| S-enantiomer of Structure XXXVI | Structure XXXVIa |
| R-enantiomer of Structure XXXVI | Structure XXXVIb |
| 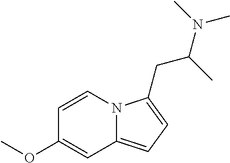 | Structure XXXVII |
| S-enantiomer of Structure XXXVII | Structure XXXVIIa |
| R-enantiomer of Structure XXXVII | Structure XXXVIIb |
| 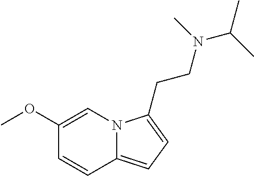 [SMILES: COc1ccc2ccc(CCN(C)C(C)C)n2c1] | Structure XXXVIII |
| 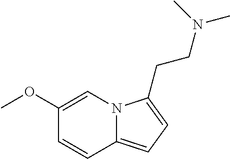 | Structure XXXIX |
| 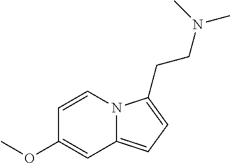 | Structure XL |
| 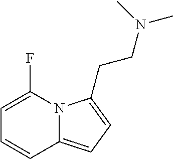 | Structure XLI |
| 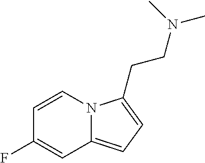 | Structure XLII |
| 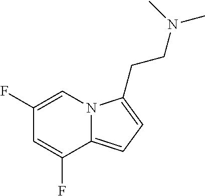 [SMILES: CN(C)CCc1ccc2c(F)cc(F)cn12] | Structure XLIII |
| 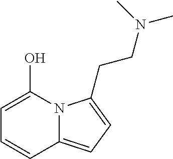 | Structure XLIV |
| 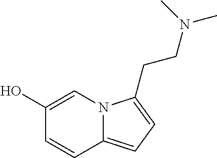 | Structure XLV |

TABLE 2
Additional Exemplary Compounds and Pure Enantiomers of the Present Invention

| Compound Structure | Compound Name |
|---|---|
| 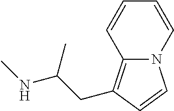 | Structure XLVI |
| S-enantiomer of Structure XLVI | Structure XLVI a |
| R-enantiomer of Structure XLVI | Structure XLVI b |
| 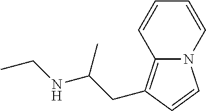 | Structure XLVII |
| S-enantiomer of Structure XLVII | Structure XLVII a |
| R-enantiomer of Structure XLVII | Structure XLVII b |
| 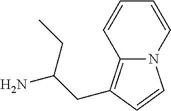 | Structure XLVIII |
| S-enantiomer of Structure XLVIII | Structure XLVIIIa |
| R-enantiomer of Structure XLVIII | Structure XLVIIIb |
| 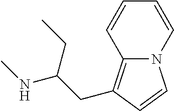 | Structure XLIX |
| S-enantiomer of Structure XLIX | Structure XLIX a |
| R-enantiomer of Structure XLIX | Structure XLIX b |
| 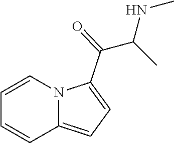 | Structure XLX |
| S-enantiomer of Structure XLX | Structure XLX a |
| R-enantiomer of Structure XLX | Structure XLX b |

In certain embodiments the S-enantiomer of a compound described above is enantiomerically enriched for example about 55:45, about 60:40, about 65:35, about 70:30, about 75:25, about 80:20, about 85:15, about 90:10, about 95:5, about 96:4, about 97:3, about 98:2, or about 99:1, or greater than about 99% S-enantiomer.

In certain embodiments the R-enantiomer of a compound described above is enantiomerically enriched for example about 55:45, about 60:40, about 65:35, about 70:30, about 75:25, about 80:20, about 85:15, about 90:10, about 95:5, about 96:4, about 97:3, about 98:2, or about 99:1, or greater than about 99% R-enantiomer.

The invention provides a compound, pure enantiomer, enantiomerically enriched mixture, or a pharmaceutically acceptable salt or salt mixture thereof of Formula I:

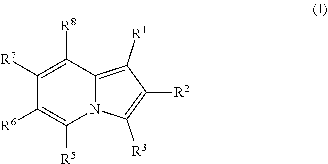

wherein:
R² is selected from R¹,

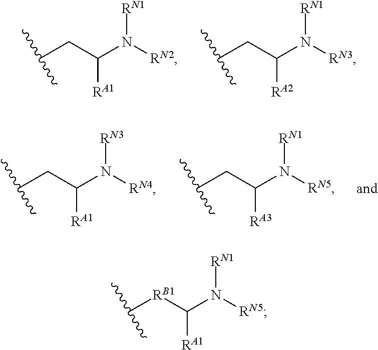

R³ is selected from R¹,

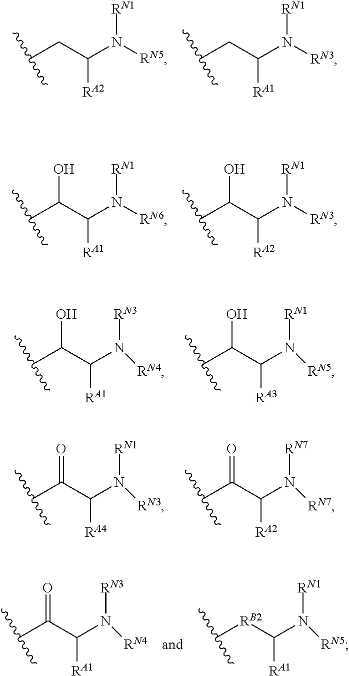

R⁵, R⁶, R⁷, and R⁸ are independently selected from R¹ and

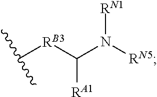

wherein 5 of the 6 of R², R³, R⁵, R⁶, R⁷, and R⁸ are R¹; and wherein each R¹ is independently selected from the group consisting of hydrogen, halogen, alkyl, haloalkyl, —OP(O)(OR⁹)₂, —SR⁹, —NR⁹R¹⁰, and —OR⁹;
R^{A1} is selected from hydrogen, —CH₃, —CH₂X, —CHX₂, —CX₃, —CH₂CH₃, —CH₂CH₂X, —CH₂CHX₂, —CH₂CX₃, —CH₂OH, and —CH₂CH₂OH;
R^{A2} is selected from —CH₃, —CH₂X, —CHX₂, —CX₃, —CH₂CH₃, —CH₂CH₂X, —CH₂CHX₂, —CH₂CX₃, —CH₂OH, and —CH₂CH₂OH;
R^{A3} is selected from —CH₂X, —CHX₂, —CX₃, —CH₂CH₃, —CH₂CH₂X, —CH₂CHX₂, —CH₂CX₃, —CH₂OH, and —CH₂CH₂OH;
R^{A4} is selected from hydrogen, —CH₂X, —CHX₂, —CX₃, —CH₂CH₃, —CH₂CH₂X, —CH₂CHX₂, —CH₂CX₃, —CH₂OH, and —CH₂CH₂OH;
R^{B1} is selected from

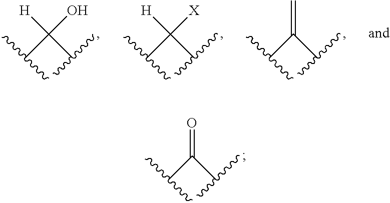

R^{B2} is selected from

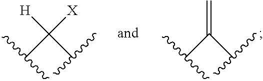

R^{B3} is selected from

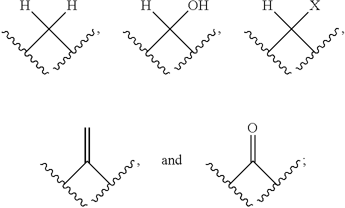

R^{N1} is independently selected in each instance from hydrogen, —(C₁-C₆)alkyl, —CH₂CH₂X, —CH₂CHX₂, —CH₂CX₃, and —CH₂CH₂OH;
R^{N2} is selected from —(C₃-C₆)alkyl, —CH₂CH₂X, —CH₂CHX₂, —CH₂CX₃, —CH₂CH₂OH, and hydroxy;
R^{N3} is selected from —(C₁-C₆)alkyl, —CH₂CH₂X, —CH₂CHX₂, —CH₂CX₃, —CH₂CH₂OH, and hydroxy;
R^{N4} is selected from —(C₁-C₆)alkyl, —CH₂CH₂X, —CH₂CHX₂, —CH₂CX₃, and —CH₂CH₂OH;
R^{N5} is independently selected in each instance from hydrogen, —(C₁-C₆)alkyl, —CH₂CH₂X, —CH₂CHX₂, —CH₂CX₃, —CH₂CH₂OH, and hydroxy;
R^{N6} is selected from —(C₂-C₆)alkyl, —CH₂CH₂X, —CH₂CHX₂, —CH₂CX₃, —CH₂CH₂OH, and hydroxy;
R^{N7} is independently selected in each instance from hydrogen, —(C₂-C₆)alkyl, —CH₂CH₂X, —CH₂CHX₂, —CH₂CX₃, —CH₂CH₂OH, and hydroxy; and
X is independently in each instance selected from —F, —Cl, —Br, and —I.

The carbon alpha to the amine is chiral when R^{A} is not hydrogen. The invention includes a compound of either the R- or S-stereochemistry at this carbon. An isolated R- or S-enantiomeric compound of the present invention can be used as a pure enantiomer or combined with the other enantiomer in any ratio that produces the desired effects. This can be an equal ratio (racemic), or in which one enantiomer is present in a greater amount than the other, referred to herein as an enantiomerically enriched mixture. Typically, in the present application, the chiral carbon referred to in the term “enantiomerically enriched” is that carbon alpha to the amine in the provided structures.

The invention additionally provides a compound of Formula II:

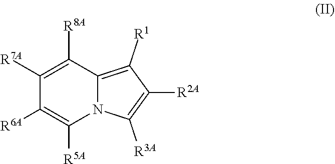

wherein:
R^{2A} is selected from R¹,

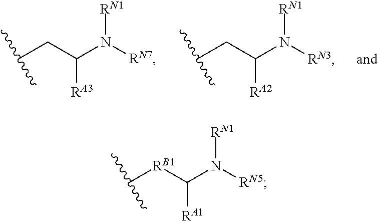

R^{3A} is selected from R¹,

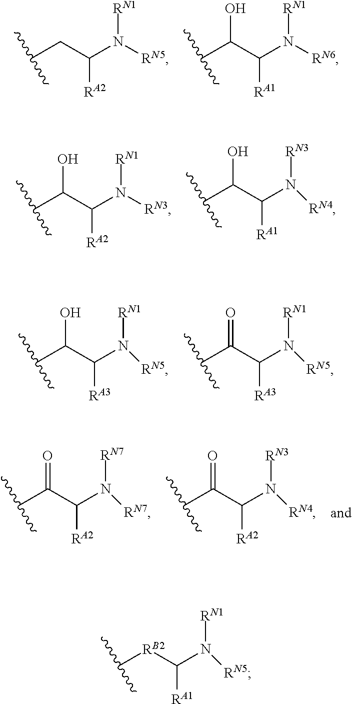

R^{5A}, R^{6A}, R^{7A}, and R^{8A} are independently selected from R¹ and

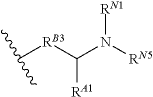

and 5 of the 6 of R^{2A}, R^{3A}, R^{5A}, R^{6A}, R^{7A}, and R^{8A} are R¹; and all other variables are as defined herein.

In some embodiments, the pure or enriched enantiomers of Formula II may be described by Formula IIb or IIc:

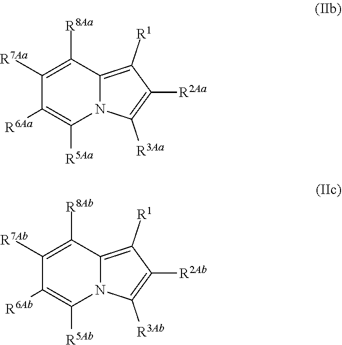

or a pharmaceutically acceptable salt or salt mixture thereof;
wherein:
R^{2Aa} is selected from R¹,

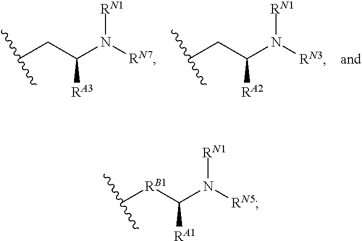

R^{2Ab} is selected from R¹,

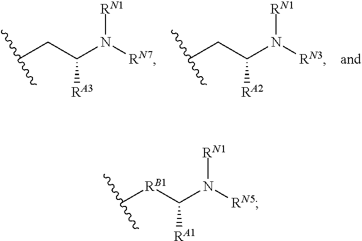

R^{3Aa} is selected from R¹,

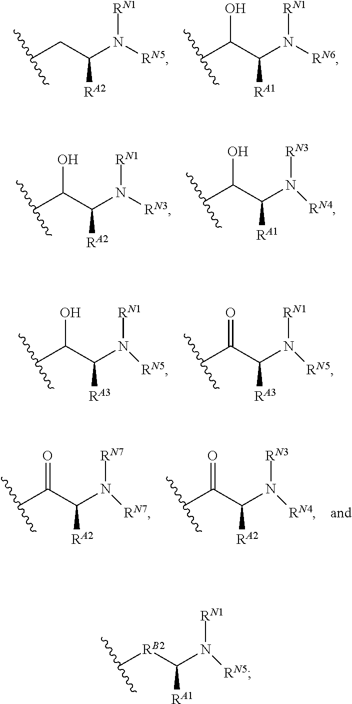

R^{3Ab} is selected from R¹,

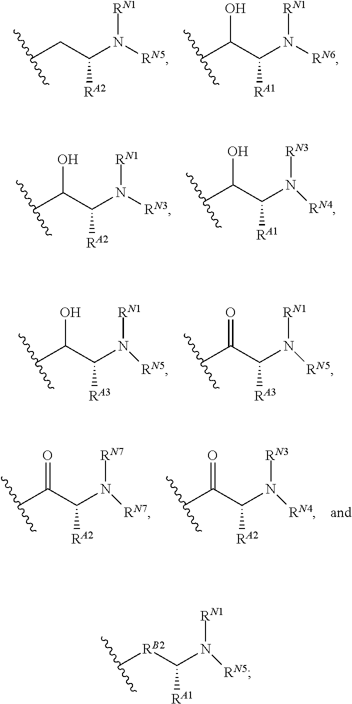

R^{5Aa}, R^{6Aa}, R^{7Aa}, and R^{8Aa} are independently selected from R¹ and

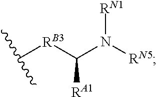

R^{5Ab}, R^{6Ab}, R^{7Ab}, and R^{8Ab} are independently selected from R¹ and

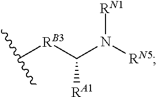

wherein 5 of the 6 of R^{2Aa}, R^{3Aa}, R^{5Aa}, R^{6Aa}, R^{7Aa}, and R^{8Aa} are R¹; and all other variables are as defined herein.

In some embodiments of the invention, a compound of Formula IIb can be combined with the other enantiomer in any ratio that produces the desired effects. This can be an equal ratio (racemic), or in the form of an enantiomerically enriched mixture in which one enantiomer is present in a greater amount than the other.

In some embodiments of the invention, a compound of Formula IIb can be combined with the other enantiomer in any ratio that produces the desired effects. This can be an equal ratio (racemic), or in the form of an enantiomerically enriched mixture in which one enantiomer is present in a greater amount than the other.

In certain embodiments, isolated enantiomers of a compound of the present invention show improved binding at the desired receptors and transporters relevant to the goal of treatment for the mental disorder or for mental enhancement.

In certain embodiments, a mixture of enantiomers of a compound of the present invention provides improved pharmacological effects and reduces unwanted effects relevant to the goal of treatment for the mental disorder or for mental enhancement.

Enantiomerically enriched mixtures of the present invention can include the enriched R-enantiomer or enriched S-enantiomer of the structures:

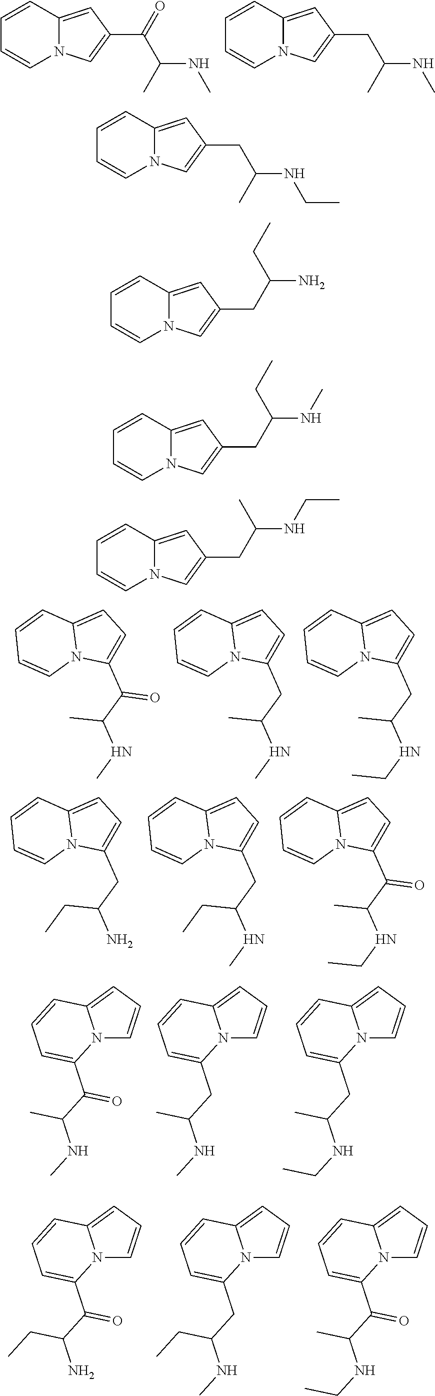

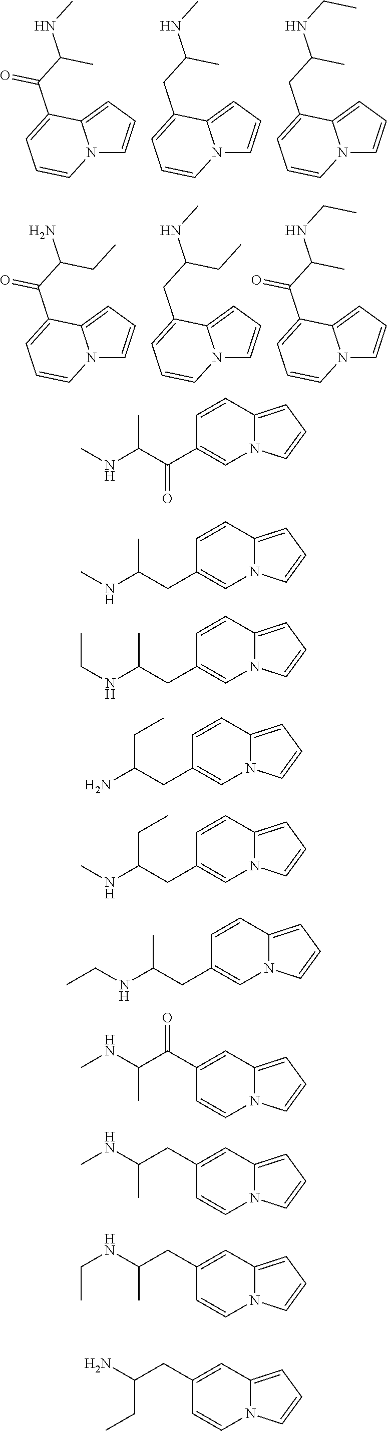

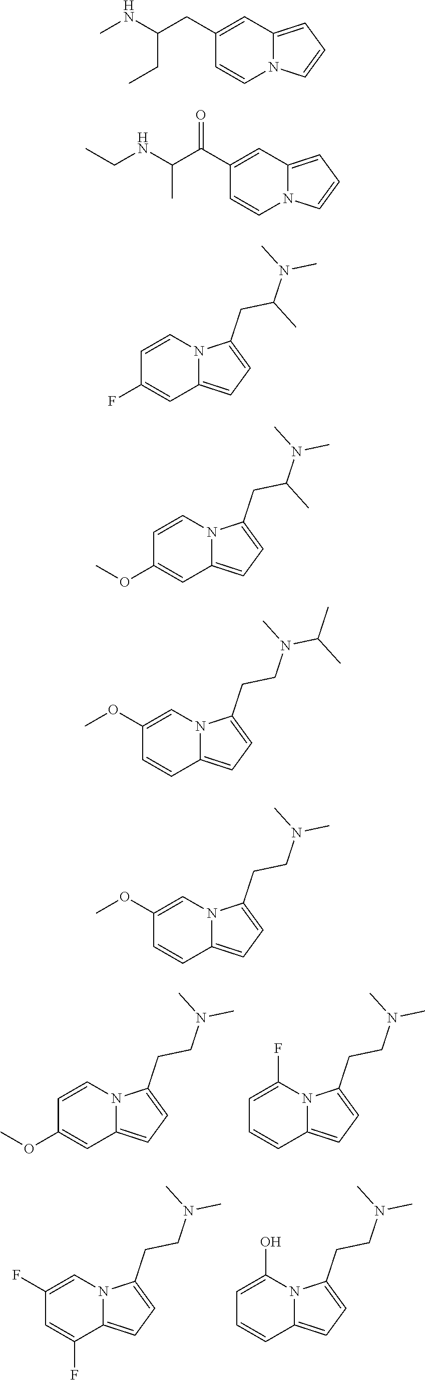

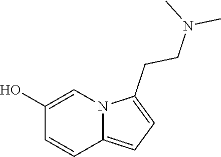

An enantiomerically enriched mixture of the present invention can additionally include a compound of Formula III:

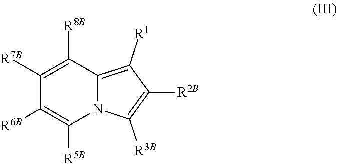

or a pharmaceutically acceptable salt or salt mixture thereof;
wherein:
R^{2B}, R^{3B}, R^{5B}, R^{6B}, R^{7B}, and R^{8B} are independently selected from R¹ and

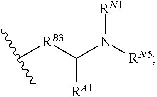

wherein 5 of the 6 of R^{2B}, R^{3B}, R^{5B}, R^{6B}, R^{7B}, and R^{8B} are not R¹; and all other variables are as defined herein.

An enantiomerically enriched mixture is a mixture that contains one enantiomer in a greater amount than the other. An enantiomerically enriched mixture of an S-enantiomer contains at least 55% of the S-enantiomer, and, typically at least about 60%, 65%, 70%, 75%, 80%, 85%, 90%, or 95% or more of the S-enantiomer. An enantiomerically enriched mixture of an R-enantiomer contains at least 55% of the R-enantiomer, and typically at least about 60%, 65%, 70%, 75%, 80%, 85%, 90% or 95% of the R-enantiomer. The specific ratio of S or R enantiomer can be selected for the need of the patient according to the health care specialist to balance the desired effect

Non-limiting examples of unwanted effects that can be minimized by carefully selecting the balance of enantiomers include hallucinogenic effects (for example, perceptual distortions, delusions, depersonalization, derealization, and labile mood), psychoactive effects (including excess stimulation or sedation), physiological effects (including transient hypertension or appetite suppression), toxic effects (including to the brain or liver), effects contributing to abuse liability (including euphoria or dopamine release), and/or other side effects.

These structures are effective for modulating serotonergic activity and producing rapid anti-neurotic effects for the treatment of CNS disorders and mental enhancement.

In certain embodiments, an enantiomerically enriched mixture of the S-enantiomer or pure enantiomer of any one of Structures I-XLV balances therapeutic effects (such as emotional openness and perceptible mood effects) while having lesser effects associated with abuse liability (such as perceptible ‘good drug effects’ or desire for more drug, which can lead to abuse; Pool et al. 2016. Neuroscience & Biobehavioral Reviews, 63, pp. 124-142) when administered to a host in need thereof, for example a mammal, including a human. The enantiomerically enriched mixture or pure enantiomer achieves a predetermined combination of emotional therapeutic effects and perceptible mood effects. The effect can be modulated as desired for optimal therapeutic effect.

In further embodiments, an indolizine compound of the current invention is a direct 5-HT_{1B} agonist. In yet further embodiments, an indolizine compound of the current invention is a 5-HT releaser. In some aspects of the present invention, an indolizine compound releases 5-HT and is a 5-HT_{1B} agonist without displaying significant toxicities.

In further embodiments, an indolizine compound of the current invention is a direct 5-HT_{2A} agonist. In yet further embodiments, an indolizine compound of the current invention is a 5-HT releaser. 5-HT_{2A} agonists increase neuroplasticity and are currently being investigated for a variety of indications, including for treating chronic pain, headache, depression, anxiety, and substance use disorders. Most substances that are 5-HT_{2A} agonists have significant side effects that are often undesirable in a therapeutic context. For example, psilocybin often produces labile mood with frequent anxiety, derealization, and depersonalization, which are signs and symptoms that limit clinical use. In some aspects of the present invention, an indolizine compound releases 5-HT and is a 5-HT_{2A} agonist while displaying greatly decreased side effects compared to psilocybin, LSD, and other clinically used 5-HT_{2A} agonists.

In other embodiments, an enantiomerically enriched mixture of the R-enantiomer or pure enantiomer of Formula I, II, or III balances therapeutic effects (such as emotional openness and perceptible mood effects) while having lesser effects associated with abuse liability (such as perceptible ‘good drug effects’ or desire for more drug, which can lead to abuse; Pool et al. 2016. Neuroscience & Biobehavioral Reviews, 63, pp. 124-142) when administered to a host in need thereof, for example a mammal, including a human. The enantiomerically enriched mixture or pure enantiomer achieves a predetermined combination of emotional therapeutic effects and perceptible mood effects. The effect can be modulated as desired for optimal therapeutic effect.

In further embodiments, an enantiomerically enriched mixture of the S-enantiomer or pure enantiomer of Formula I, II, or III balances therapeutic effects (such as emotional openness and perceptible mood effects) while having lesser effects associated with abuse liability (such as perceptible ‘good drug effects’ or desire for more drug, which can lead to abuse; Pool et al. 2016. Neuroscience & Biobehavioral Reviews, 63, pp. 124-142) when administered to a host in need thereof, for example a mammal, including a human. The enantiomerically enriched mixture or pure enantiomer achieves a predetermined combination of emotional therapeutic effects and perceptible mood effects. The effect can be modulated as desired for optimal therapeutic effect.

The present invention also provides new medical uses for the described compounds, including but not limited to, administration in an effective amount to a host in need thereof such as a human for post-traumatic stress disorder, depression, dysthymia, anxiety, generalized anxiety, social anxiety, panic, adjustment disorder, feeding and eating disorders, binge behaviors, body dysmorphic syndromes, addiction, drug abuse or dependence disorders, substance use disorders, disruptive behavior disorders, impulse control disorders, gaming disorders, gambling disorders, memory loss, dementia of aging, attention deficit hyperactivity disorder, personality disorders, attachment disorders, autism or dissociative disorders or any other disorder described herein, including in the Background. One particular treatment is for adjustment disorder, which is highly prevalent in society and currently insufficiently addressed. In nonlimiting aspects, the compound used in the treatment includes, for example, a racemic compound, pure enantiomer, or enantiomerically enriched composition of R- or S-enantiomer of Formula I, II, or III, Structures I-XLV, or a combination thereof. In nonlimiting aspects, the compound used in the treatment includes, for example, a compound of Formula I.

In some embodiments a compound of the present invention has pharmacokinetic properties suitable for administration to a mammal, for example a human. Beneficial pharmacokinetic properties can include having less variable pharmacokinetic properties than MDMA. In certain embodiments, a compound of the present invention has a more consistent maximum plasma concentration (Cₘₐₓ) than MDMA at a given dose. In certain embodiments, a compound of the present invention has a more consistent area-under-the-concentration-versus-time-curve (AUC) than MDMA for a given dosage regimen. An additional beneficial property that can be demonstrated by a compound of the present invention is less inhibition of CYP enzymes compared to MDMA. Inhibition of CYP enzymes can cause toxic drug-drug interactions. In certain embodiments, a compound of the present invention does not inhibit or shows minimal inhibition of cytochrome p450 isozyme 2D6 (CYP2D6). In certain embodiments, a compound of the present invention shows less potent inhibition of CYP2D6 than MDMA.

A disclosed compound can be used in an effective amount to improve neurological or psychiatric functioning in a patient in need thereof. Neurological indications include, but are not limited to, improved neuroplasticity, including treatment of stroke, brain trauma, dementia, and neurodegenerative diseases. MDMA has an EC₅₀ of 7.41 nM for promoting neuritogenesis and an Emax approximately twice that of ketamine, which has fast acting psychiatric benefits that are thought to be mediated by its ability to promote neuroplasticity, including the growth of dendritic spines, increased synthesis of synaptic proteins, and strengthening synaptic responses (Ly et al. Cell reports 23, no. 11 (2018): 3170-3182; Figure S3). A compound of the current invention can similarly be considered a psychoplastogen, that is, small molecules that are able to induce rapid neuroplasticity (Olson, 2018, Journal of experimental neuroscience, 12, 1179069518800508). For example, in certain embodiments, a disclosed compound or composition can be used to improve stuttering and other dyspraxias or to treat Parkinson's disease or schizophrenia.

In certain embodiments, the compound of the present invention is selected from:

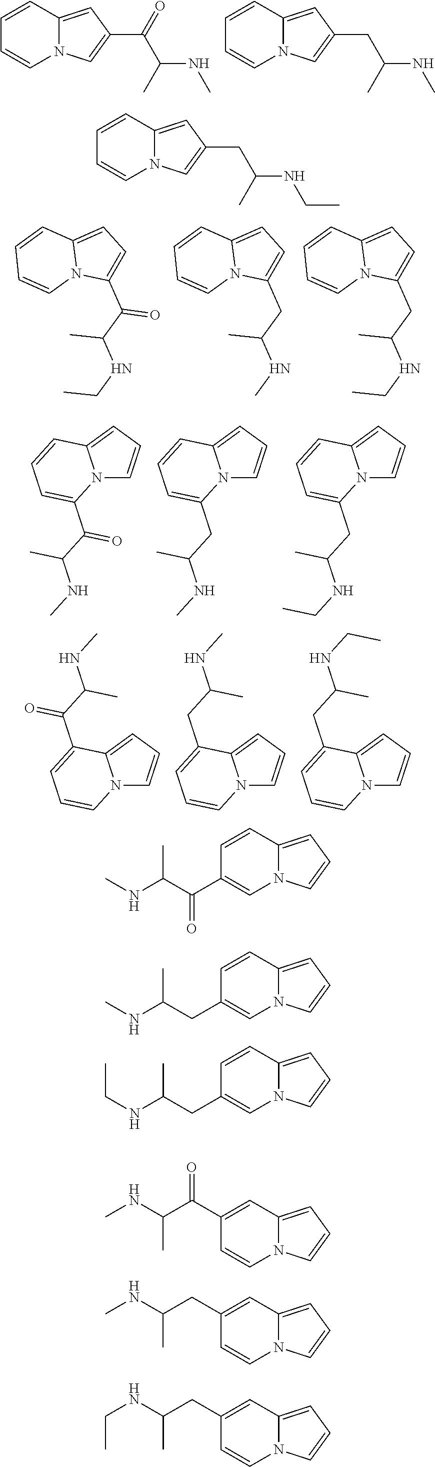

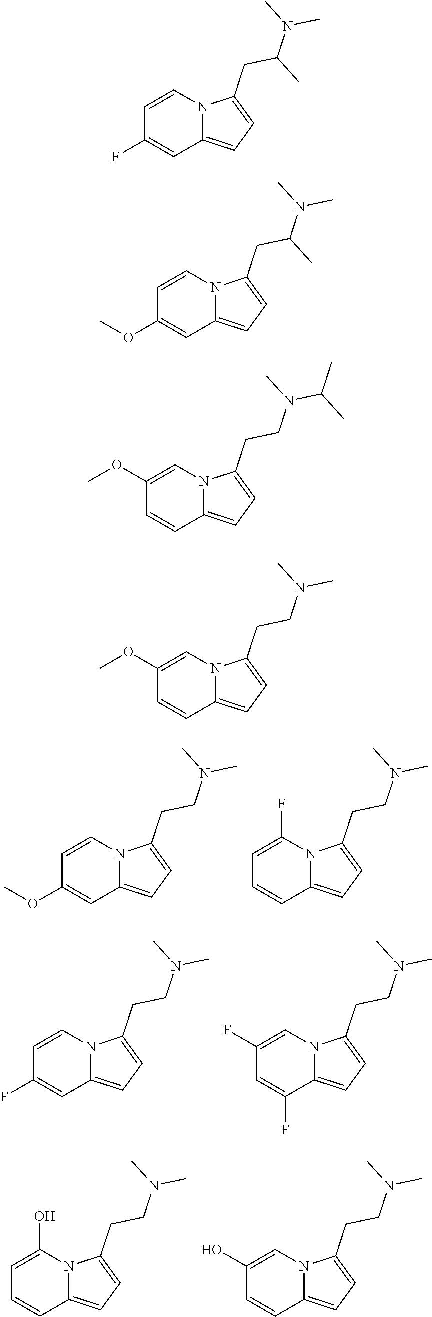

or an enantiomer or mixture of enantiomers or a pharmaceutically acceptable salt or salt mixture thereof.

In certain embodiments, the compound of the present invention is selected from Table 1. A compound in Table 1 is also considered optionally as a pharmaceutically acceptable salt or salt mixture thereof.

### ADDITIONAL EMBODIMENTS OF THE PRESENT INVENTION

In certain embodiments the compound of the present invention is selected from:

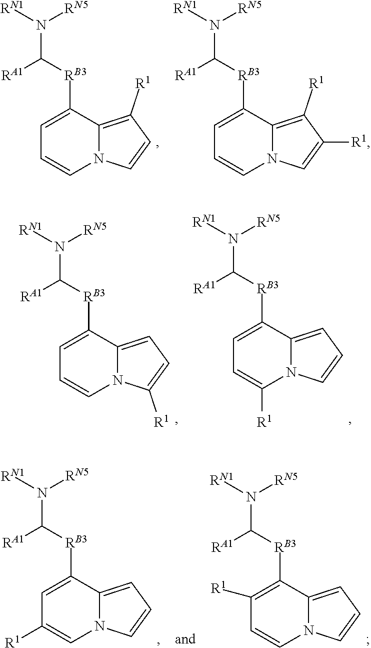

or a pharmaceutically acceptable salt or salt mixture thereof.

In certain embodiments the compound of the present invention is selected from:

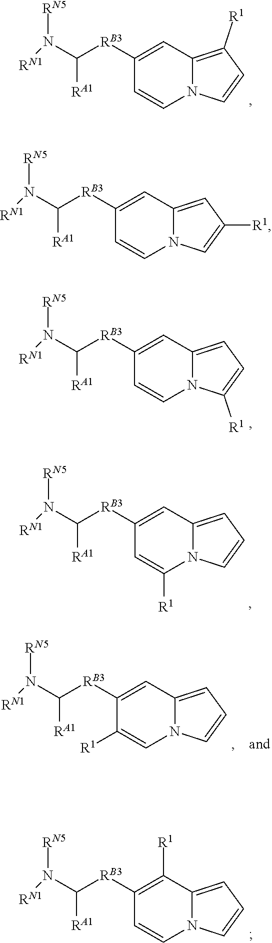

or a pharmaceutically acceptable salt or salt mixture thereof.

In certain embodiments the compound of the present invention is selected from:

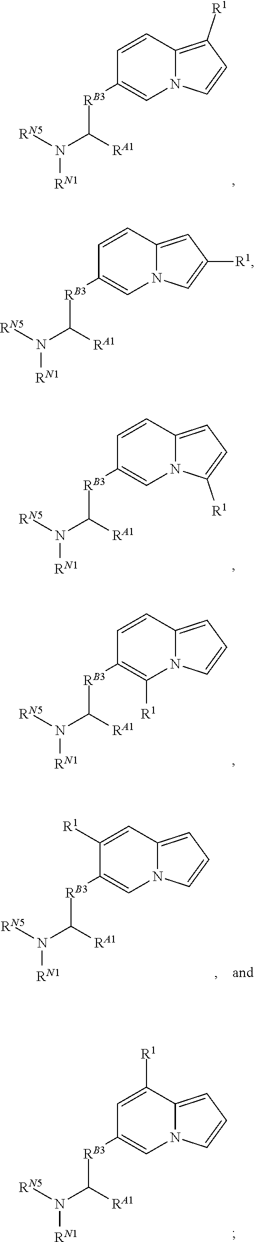

or a pharmaceutically acceptable salt or salt mixture thereof.

In certain embodiments the compound of the present invention is selected from:

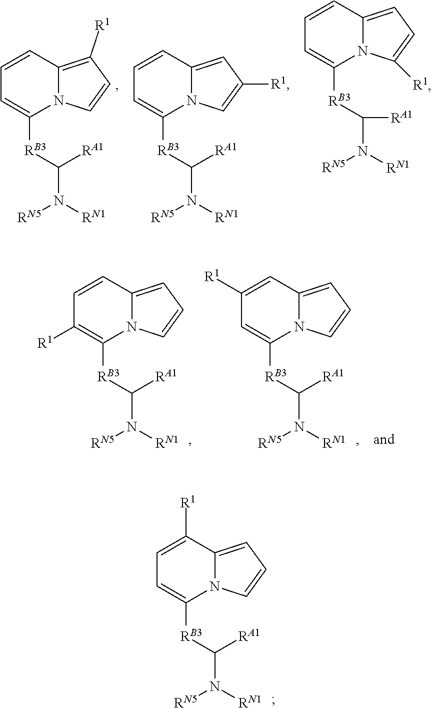

or a pharmaceutically acceptable salt or salt mixture thereof.

In certain embodiments the compound of the present invention is selected from:

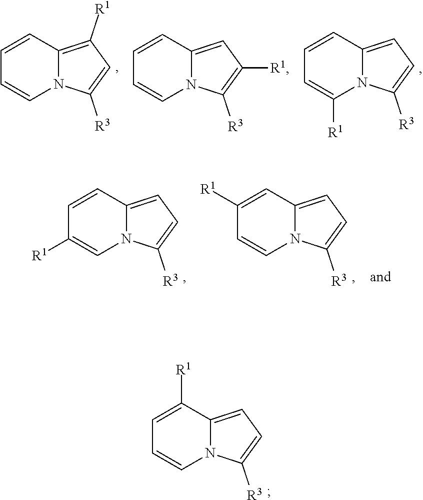

or a pharmaceutically acceptable salt or salt mixture thereof;
wherein R³ is selected from

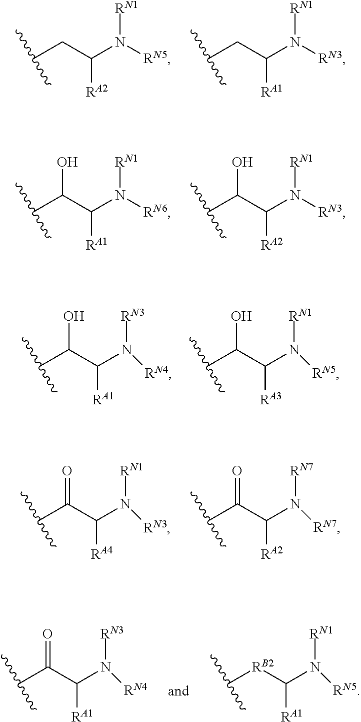

In certain embodiments the compound of the present invention is selected from:

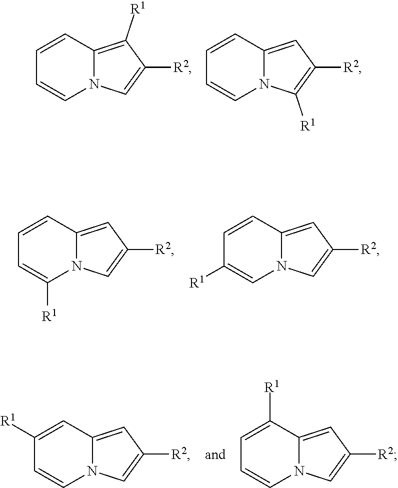

or a pharmaceutically acceptable salt or salt mixture thereof;
wherein R² is selected from

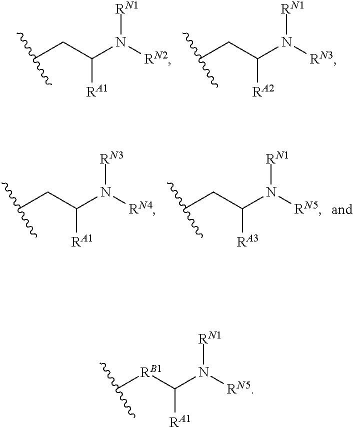

In certain embodiments the compound of the present invention is of Formula:

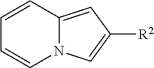

or a pharmaceutically acceptable salt or salt mixture thereof;
wherein R² is selected from

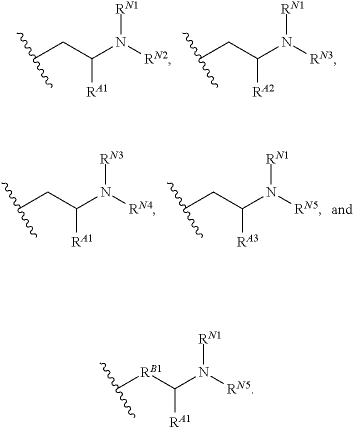

Additional exemplary compounds of the present invention include

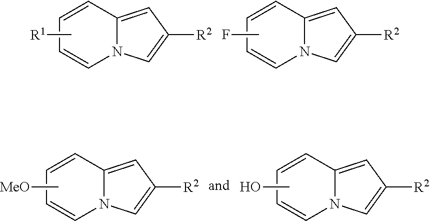

or a pharmaceutically acceptable salt or salt mixture thereof.

Additional exemplary compounds of the present invention include

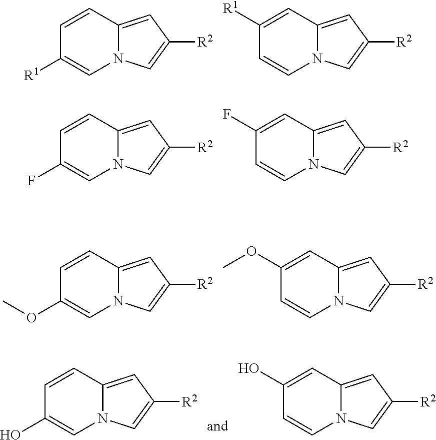

or a pharmaceutically acceptable salt or salt mixture thereof.

Further exemplary compounds of the present invention include

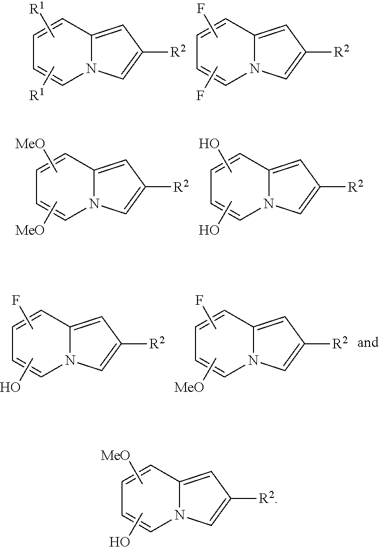

or a pharmaceutically acceptable salt or salt mixture thereof.

In certain embodiments the compound of the present invention is of Formula:

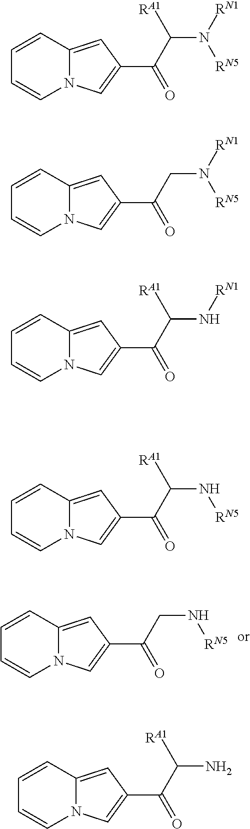

or a pharmaceutically acceptable salt or salt mixture thereof.

In certain embodiments the compound of the present invention is of Formula:

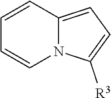

or a pharmaceutically acceptable salt or salt mixture thereof;
wherein R³ is selected from

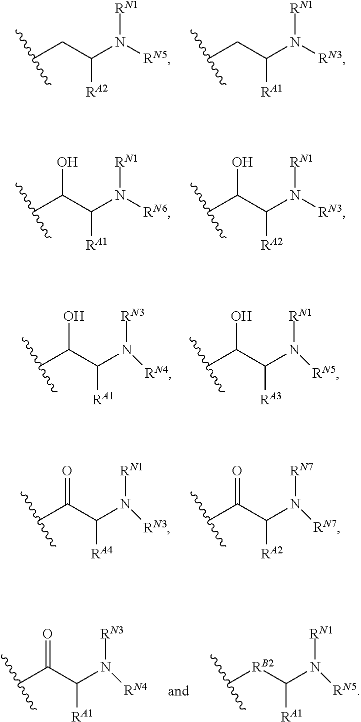

Additional exemplary compounds of the present invention include

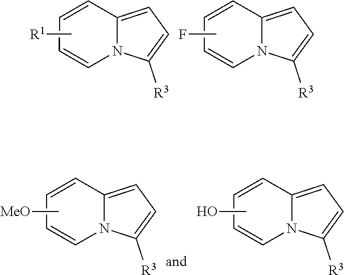

or a pharmaceutically acceptable salt or salt mixture thereof.

Additional exemplary compounds of the present invention include

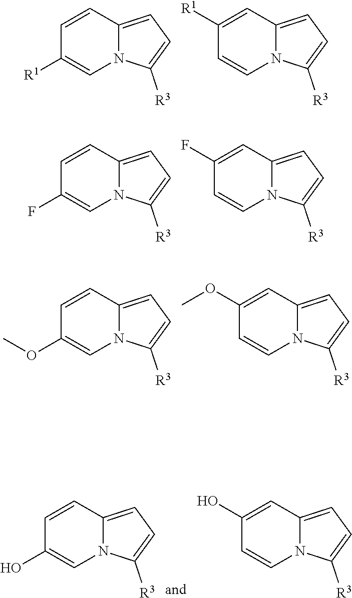

or a pharmaceutically acceptable salt or salt mixture thereof.

Further exemplary compounds of the present invention include

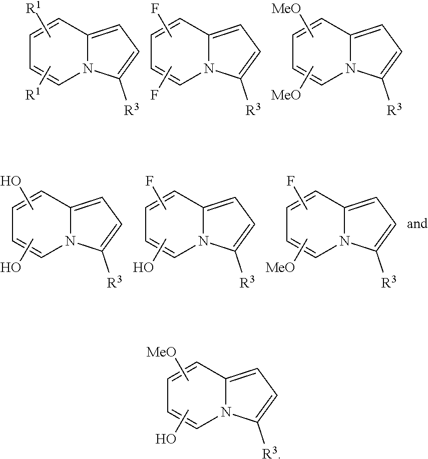

or a pharmaceutically acceptable salt or salt mixture thereof.

In certain embodiments the compound of the present invention is of Formula:

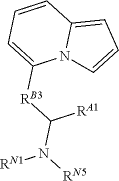

or a pharmaceutically acceptable salt or salt mixture thereof.

Additional exemplary compounds of the present invention include

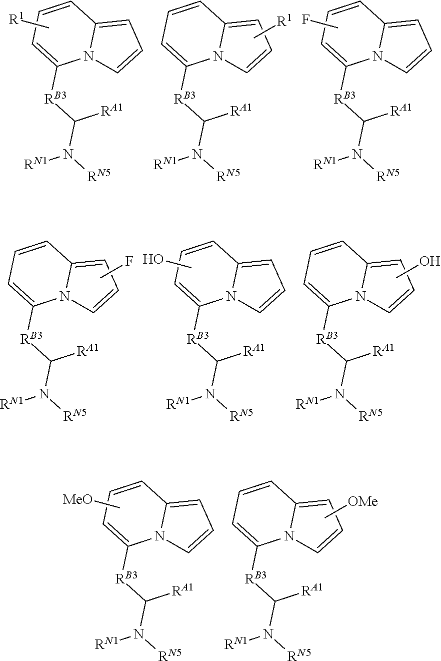

or a pharmaceutically acceptable salt or salt mixture thereof.

Additional exemplary compounds of the present invention include

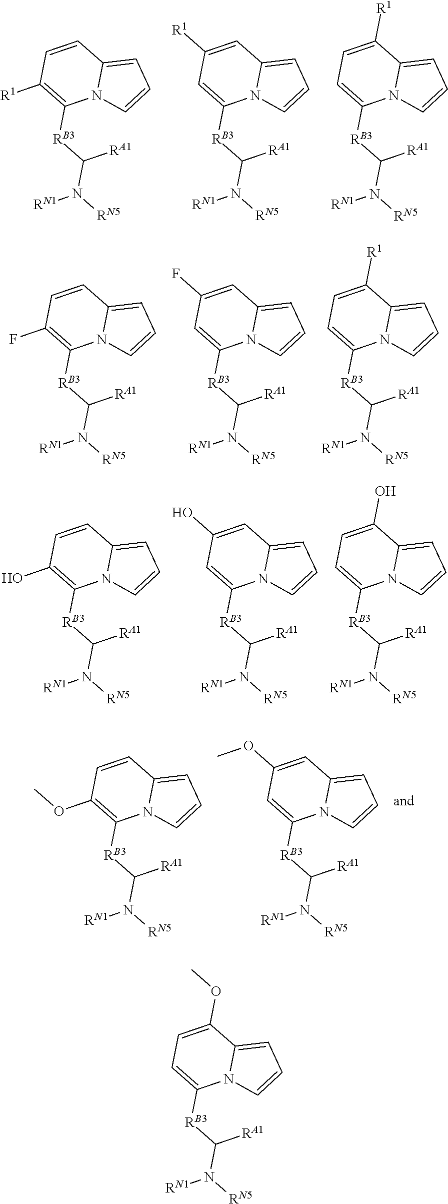

or a pharmaceutically acceptable salt or salt mixture thereof.

Further exemplary compounds of the present invention include

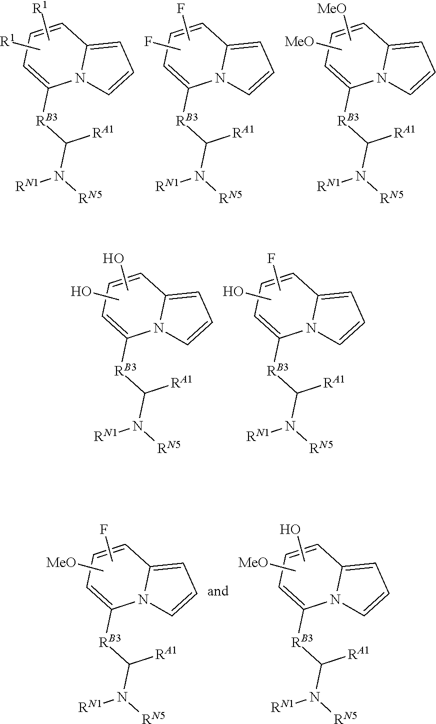

or a pharmaceutically acceptable salt or salt mixture thereof.

In certain embodiments the compound of the present invention is of Formula:

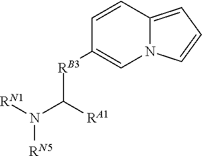

or a pharmaceutically acceptable salt or salt mixture thereof.

Additional exemplary compounds of the present invention include

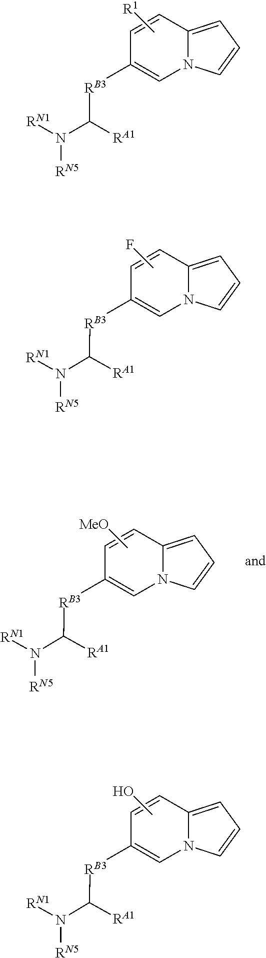

or a pharmaceutically acceptable salt or salt mixture thereof.

Additional exemplary compounds of the present invention include

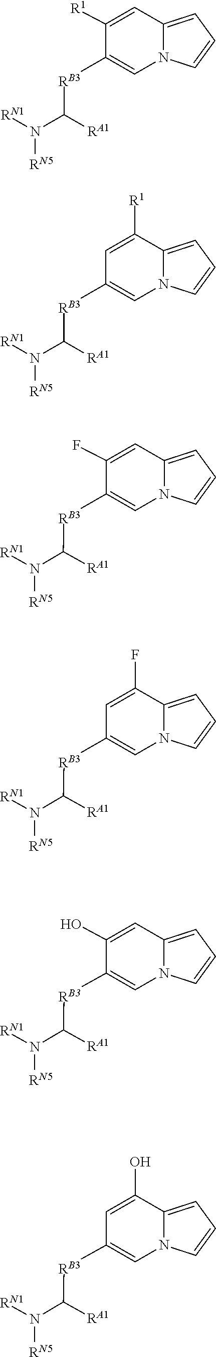

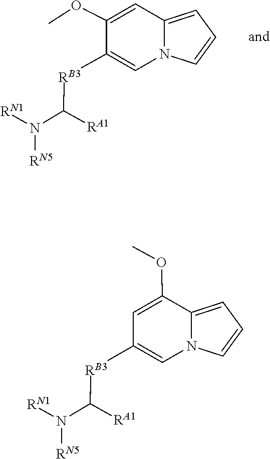

or a pharmaceutically acceptable salt or salt mixture thereof.

Further exemplary compounds of the present invention include

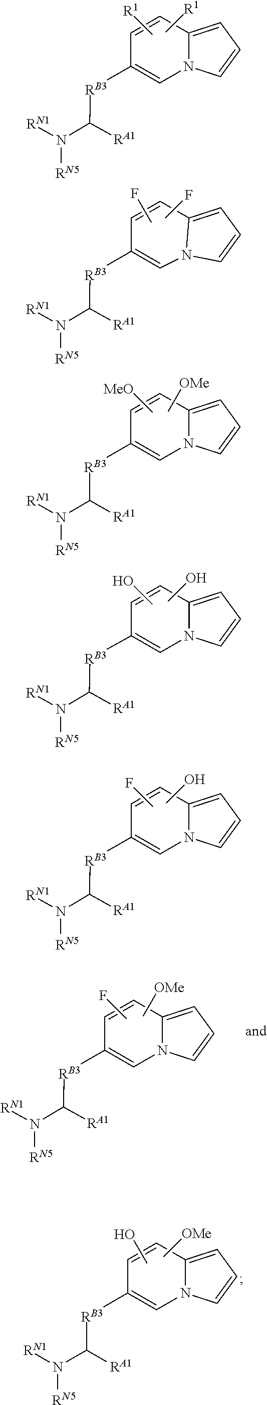

or a pharmaceutically acceptable salt or salt mixture thereof.

In certain embodiments the compound of the present invention is of Formula:

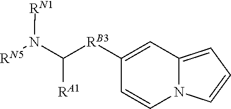

or a pharmaceutically acceptable salt or salt mixture thereof.

Additional exemplary compounds of the present invention include

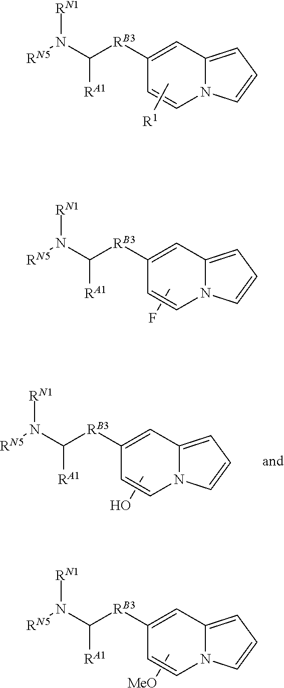

or a pharmaceutically acceptable salt or salt mixture thereof.

Additional exemplary compounds of the present invention include

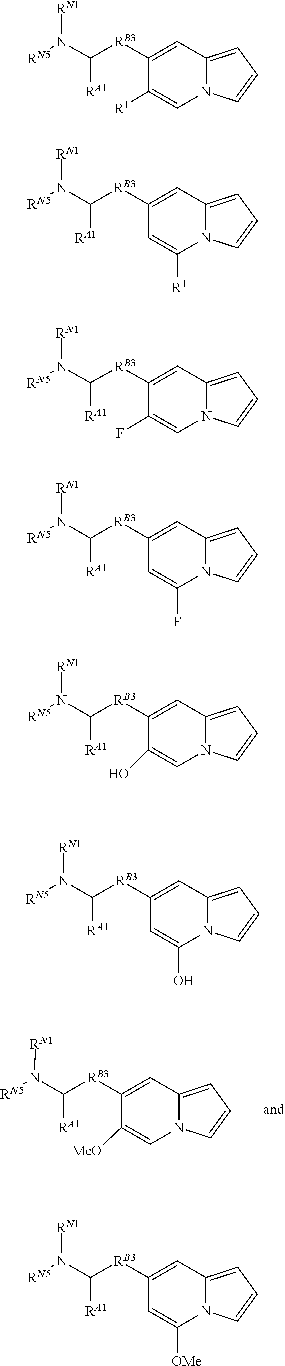

or a pharmaceutically acceptable salt or salt mixture thereof.

Further exemplary compounds of the present invention include

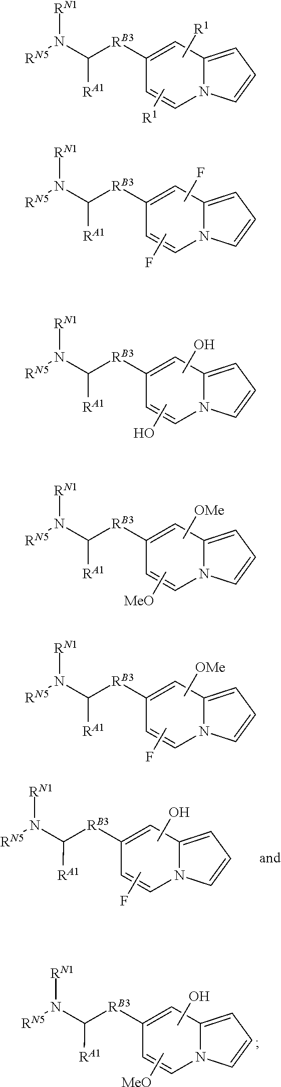

or a pharmaceutically acceptable salt or salt mixture thereof.

In certain embodiments the compound of the present invention is of Formula:

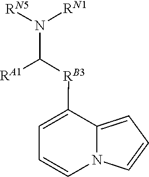

or a pharmaceutically acceptable salt or salt mixture thereof.

Additional exemplary compounds of the present invention include

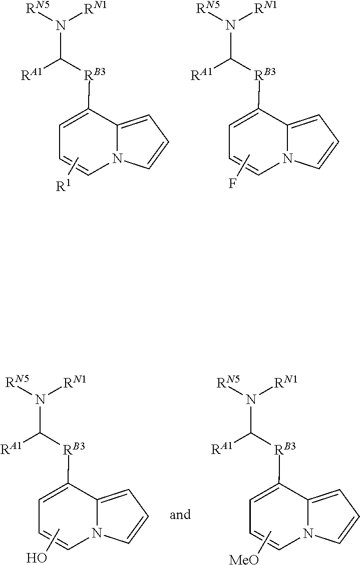

or a pharmaceutically acceptable salt or salt mixture thereof.

Additional exemplary compounds of the present invention include

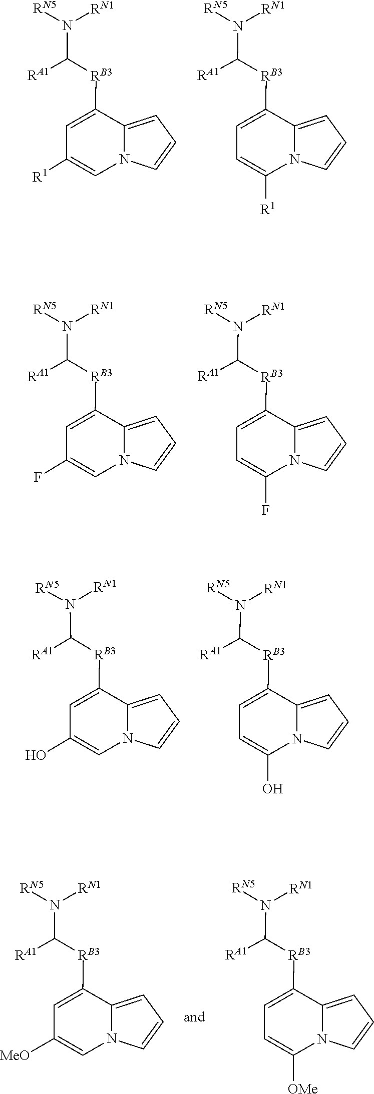

or a pharmaceutically acceptable salt or salt mixture thereof.

Further exemplary compounds of the present invention include

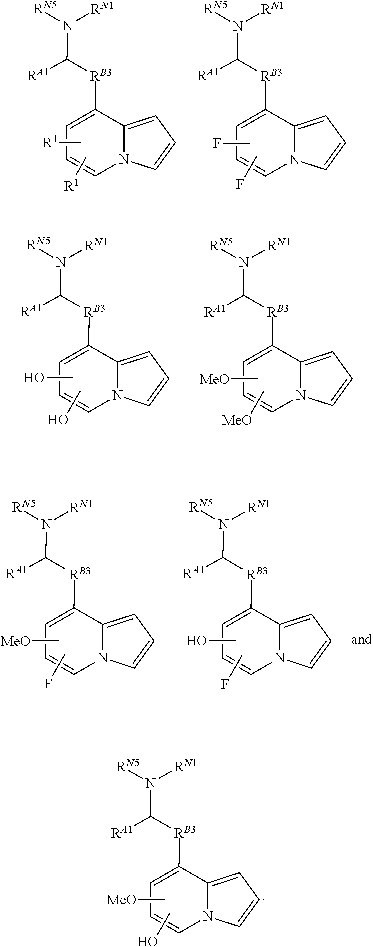

### Embodiments of “Alkyl”

In certain embodiments “alkyl” is a branched, straight chain, or cyclic saturated aliphatic hydrocarbon group. In certain embodiments, the alkyl from 1 to about 6 carbon atoms, from 1 to about 4 carbon atoms, or from 1 to 3 carbon atoms. In certain embodiments, the alkyl contains from 1 to about 8 carbon atoms. In certain embodiments, the alkyl is C₁-C₂, C₁-C₃, C₁-C₄, C₁-C₅ or C₁-C₆. The specified ranges as used herein indicate an alkyl group which is considered to explicitly disclose as individual species each member of the range described as a unique species. For example, the term C₁-C₆ alkyl as used herein indicates a straight or branched alkyl group having 1, 2, 3, 4, 5, or 6 carbon atoms and also a carbocyclic alkyl group of 3, 4, 5, or 6 carbon atoms and is intended to mean that each of these is described as an independent species. For example, the term C₁-C₄alkyl as used herein indicates a straight or branched alkyl group having 1, 2, 3, or 4 carbon atoms and is intended to mean that each of these is described as an independent species. Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, 2,3-dimethylbutane, and hexyl.

In certain embodiments “alkyl” is a C₁-C₆alkyl, C₁-C₅alkyl, C₁-C₄alkyl, C₁-C₃alkyl, or C₁-C₂alkyl.

In certain embodiments “alkyl” has one carbon.

In certain embodiments “alkyl” has two carbons.

In certain embodiments “alkyl” has three carbons.

In certain embodiments “alkyl” has four carbons.

In certain embodiments “alkyl” has five carbons.

In certain embodiments “alkyl” has six carbons.

Non-limiting examples of “alkyl” include: methyl, ethyl, propyl, butyl, pentyl, and hexyl.

Additional non-limiting examples of “alkyl” include: isopropyl, isobutyl, isopentyl, and isohexyl.

Additional non-limiting examples of “alkyl” include: sec-butyl, sec-pentyl, and sec-hexyl.

Additional non-limiting examples of “alkyl” include: tert-butyl, tert-pentyl, and tert-hexyl.

Additional non-limiting examples of “alkyl” include: neopentyl, 3-pentyl, and active pentyl.

In certain embodiments when a term is used that includes “alk” it should be understood that “cycloalkyl” or “carbocyclic” can be considered part of the definition, unless unambiguously excluded by the context. For example, and without limitation, the terms alkyl, alkenyl, alkynyl, alkoxy, alkanoyl, alkenloxy, haloalkyl, etc. can all be considered to include the cyclic forms of alkyl, unless unambiguously excluded by context.

### Embodiments of “Haloalkyl”

In certain embodiments “haloalkyl” indicates both branched and straight-chain alkyl groups substituted with one or more halogen atoms, up to the maximum allowable number of halogen atoms. Examples of haloalkyl include, but are not limited to, trifluoromethyl, monofluoromethyl, difluoromethyl, 2-fluoroethyl, and penta-fluoroethyl.

In certain embodiments “haloalkyl” is a C₁-C₆haloalkyl, C₁-C₅haloalkyl, C₁-C₄haloalkyl, C₁-C₃haloalkyl, and C₁-C₂haloalkyl.

In certain embodiments “haloalkyl” has one carbon.

In certain embodiments “haloalkyl” has one carbon and one halogen.

In certain embodiments “haloalkyl” has one carbon and two halogens.

In certain embodiments “haloalkyl” has one carbon and three halogens.

In certain embodiments “haloalkyl” has two carbons.

In certain embodiments “haloalkyl” has three carbons.

In certain embodiments “haloalkyl” has four carbons.

In certain embodiments “haloalkyl” has five carbons.

In certain embodiments “haloalkyl” has six carbons.

Non-limiting examples of “haloalkyl” include:

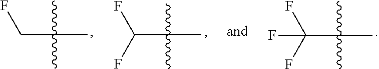

Additional non-limiting examples of “haloalkyl” include:

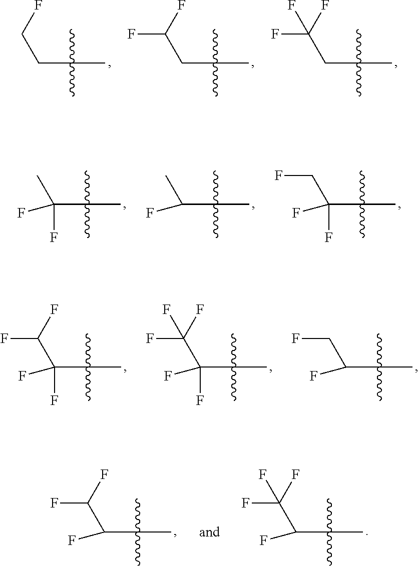

Additional non-limiting examples of “haloalkyl” include:

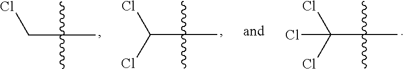

Additional non-limiting examples of “haloalkyl” include:

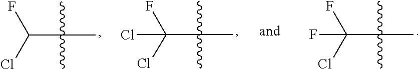

### Embodiments of R¹

In certain embodiments every R¹ is hydrogen.

In certain embodiments two R¹ groups are hydrogen.

In certain embodiments three R¹ groups are hydrogen.

In certain embodiments four R¹ groups are hydrogen.

In certain embodiments five R¹ groups are hydrogen.

In certain embodiments one R¹ is halogen.

In certain embodiments one R¹ is —F.

In certain embodiments one R¹ is —Cl.

In certain embodiments one R¹ is —Br.

In certain embodiments one R¹ is —I.

In certain embodiments one R¹ is alkyl.

In certain embodiments one R¹ is methyl.

In certain embodiments one R¹ is ethyl.

In certain embodiments one R¹ is n-propyl.

In certain embodiments one R¹ is isopropyl.

In certain embodiments one R¹ is haloalkyl.

In certain embodiments one R¹ is —CF₃.

In certain embodiments one R¹ is —OP(O)(OR⁹)₂.

In certain embodiments one R¹ is —OP(O)(OH)₂.

In certain embodiments one R¹ is —SR⁹.

In certain embodiments one R¹ is —SH.

In certain embodiments one R¹ is —SCF₃.

In certain embodiments one R¹ is —SMe.

In certain embodiments one R¹ is —NR⁹R¹⁰.

In certain embodiments one R¹ is —NHR¹⁰.

In certain embodiments one R¹ is —NH₂.

In certain embodiments one R¹ is —NHMe.

In certain embodiments one R¹ is —N(Me)₂.

In certain embodiments one R¹ is —OR⁹.

In certain embodiments one R¹ is —OH.

In certain embodiments one R¹ is —OCF₃.

In certain embodiments one R¹ is —OCH₃.

In certain embodiments two R¹ groups are —F.

In certain embodiments two R¹ groups are —CH₃.

certain embodiments two R¹ groups are —OCH₃.

In certain embodiments three R¹ groups are —F.

In certain embodiments three R¹ groups are —CH₃.

certain embodiments three R¹ groups are —OCH₃.

In certain embodiments R¹ is selected from hydrogen, F, CH₃, and —OMe.

### Embodiments of R²

In certain embodiments R² is not R¹.

In certain embodiments R² is hydrogen.

In certain embodiments R² is

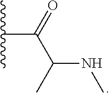 [SMILES: *C(=O)C(C)NC]

In certain embodiments R² is

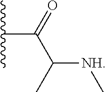 [SMILES: *C(=O)C(C)NC]

In certain embodiments R² is

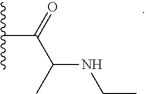 [SMILES: *C(=O)C(C)NCC]

In certain embodiments R² is

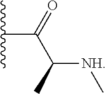 [SMILES: *C(=O)[C@H](C)NC]

In certain embodiments R² is

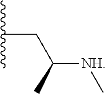 [SMILES: *C[C@H](C)NC]

In certain embodiments R² is

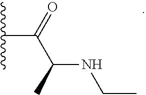 [SMILES: *C(=O)[C@H](C)NCC]

In certain embodiments R² is

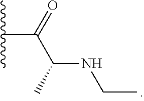 [SMILES: *C(=O)[C@@H](C)NCC]

In certain embodiments R² is

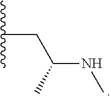 [SMILES: *C[C@@H](C)NC]

In certain embodiments R² is

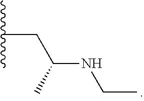 [SMILES: *C[C@@H](C)NCC]

In certain embodiments R² is

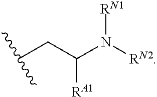

In certain embodiments R² is

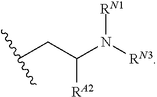

In certain embodiments R² is

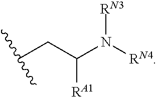

In certain embodiments R² is

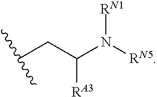

In certain embodiments R² is

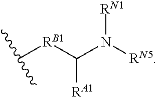

In certain embodiments R² is any one of the embodiments above and R¹, R³, R⁵, R⁶, R⁷ and R⁸ are each hydrogen.

### Embodiments of R³

In certain embodiments R³ is not R¹.

In certain embodiments R³ is hydrogen.

In certain embodiments R³ is

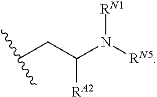

In certain embodiments R³ is

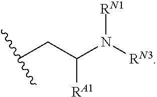

In certain embodiments R³ is

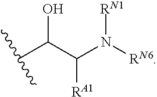

In certain embodiments R³ is

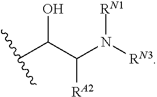

In certain embodiments R³ is

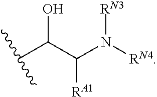

In certain embodiments R³ is

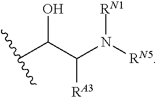

In certain embodiments R³ is

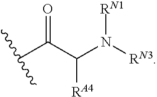

In certain embodiments R³ is

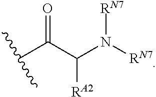

In certain embodiments R³ is

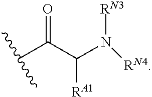

In certain embodiments R³ is

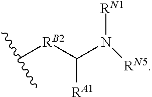

In certain embodiments R³ is any one of the embodiments above and R¹, R², R⁵, R⁶, R⁷ and R⁸ are each hydrogen.

### Embodiments of R⁵

In certain embodiments R⁵ is not R¹.

In certain embodiments R⁵ is hydrogen.

In certain embodiments R⁵ is

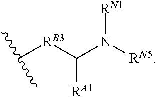

In certain embodiments R⁵ is

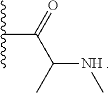 [SMILES: *C(=O)C(C)NC]

In certain embodiments R⁵ is

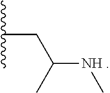 [SMILES: *CC(C)NC]

In certain embodiments R⁵ is

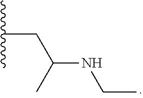 [SMILES: *CC(C)NCC]

In certain embodiments R⁵ is

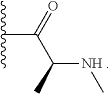 [SMILES: *C(=O)[C@H](C)NC]

In certain embodiments R⁵ is

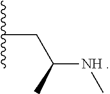 [SMILES: *C[C@H](C)NC]

In certain embodiments R⁵ is

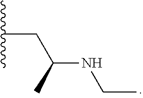 [SMILES: *C[C@H](C)NCC]

In certain embodiments R⁵ is

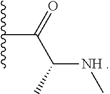 [SMILES: *C(=O)[C@@H](C)NC]

In certain embodiments R⁵ is

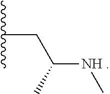 [SMILES: *C[C@@H](C)NC]

In certain embodiments R⁵ is

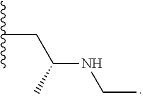 [SMILES: *C[C@@H](C)NCC]

In certain embodiments R⁵ is any one of the embodiments above and R¹, R², R³, R⁶, R⁷ and R⁸ are each hydrogen.

### Embodiments of R⁶

In certain embodiments R⁶ is not R¹.

In certain embodiments R⁶ is hydrogen.

In certain embodiments R⁶ is

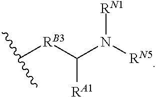

In certain embodiments R⁶ is

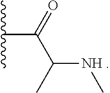 [SMILES: *C(=O)C(C)NC]

In certain embodiments R⁶ is

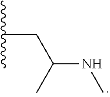 [SMILES: *CC(C)NC]

In certain embodiments R⁶ is

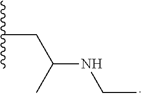 [SMILES: *CC(C)NCC]

In certain embodiments R⁶ is

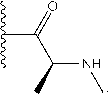 [SMILES: *C(=O)[C@H](C)NC]

In certain embodiments R⁶ is

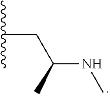 [SMILES: *C[C@H](C)NC]

In certain embodiments R⁶ is

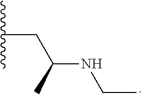 [SMILES: *C[C@H](C)NCC]

In certain embodiments R⁶ is

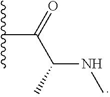 [SMILES: *C(=O)[C@@H](C)NC]

In certain embodiments R⁶ is

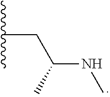 [SMILES: *C[C@@H](C)NC]

In certain embodiments R⁶ is

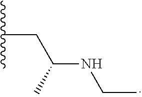 [SMILES: *C[C@@H](C)NCC]

In certain embodiments R⁶ is any one of the embodiments above and R¹, R², R³, R⁵, R⁷, and R⁸ are each hydrogen.

### Embodiments of R⁷

In certain embodiments R⁷ is not R¹.

In certain embodiments R⁷ is hydrogen.

In certain embodiments R⁷ is

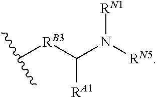

In certain embodiments R⁷ is

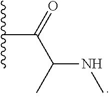 [SMILES: *C(=O)C(C)NC]

In certain embodiments R⁷ is

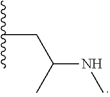 [SMILES: *CC(C)NC]

In certain embodiments R⁷ is

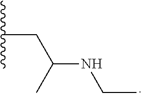 [SMILES: *CC(C)NCC]

In certain embodiments R⁷ is

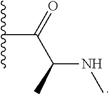 [SMILES: *C(=O)[C@H](C)NC]

In certain embodiments R⁷ is

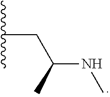 [SMILES: *C[C@H](C)NC]

In certain embodiments R⁷ is

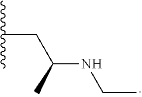 [SMILES: *C[C@H](C)NCC]

In certain embodiments R⁷ is

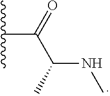 [SMILES: *C(=O)[C@@H](C)NC]

In certain embodiments R⁷ is

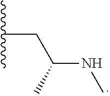 [SMILES: *C[C@@H](C)NC]

In certain embodiments R⁷ is

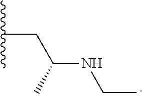 [SMILES: *C[C@@H](C)NCC]

In certain embodiments R⁷ is any one of the embodiments above and R¹, R², R³, R⁵, R⁶ and R⁸ are each hydrogen.

### Embodiments of R⁸

In certain embodiments R⁸ is not R¹.

In certain embodiments R⁸ is hydrogen.

In certain embodiments R⁸ is

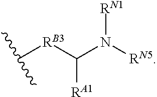

In certain embodiments R⁹ is

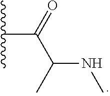 [SMILES: *C(=O)C(C)NC]

In certain embodiments R⁹ is

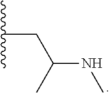 [SMILES: *CC(C)NC]

In certain embodiments R⁹ is

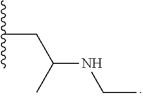 [SMILES: *CC(C)NCC]

In certain embodiments R⁸ is

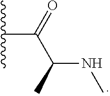 [SMILES: *C(=O)[C@H](C)NC]

In certain embodiments R⁸ is

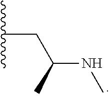 [SMILES: *C[C@H](C)NC]

In certain embodiments R⁸ is

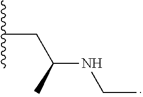 [SMILES: *C[C@H](C)NCC]

In certain embodiments R⁸ is

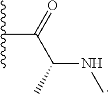 [SMILES: *C(=O)[C@@H](C)NC]

In certain embodiments R⁸ is

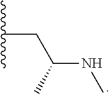 [SMILES: *C[C@@H](C)NC]

In certain embodiments R⁸ is

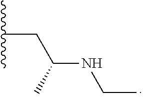 [SMILES: *C[C@@H](C)NCC]

In certain embodiments R⁸ is any one of the embodiments above and R¹, R², R³, R⁵, R⁶ and R⁷ are each hydrogen.

### Embodiments of R^{A1}

In certain embodiments R^{A1} is hydrogen.

In certain embodiments R^{A1} is —CH₃.

In certain embodiments R^{A1} is —CH₂X.

In certain embodiments R^{A1} is —CHX₂.

In certain embodiments R^{A1} is —CX₃.

In certain embodiments R^{A1} is —CH₂CH₃.

In certain embodiments R^{A1} is —CH₂CH₂X.

In certain embodiments R^{A1} is —CH₂CHX₂.

In certain embodiments R^{A1} is —CH₂CX₃.

In certain embodiments R^{A1} is —CH₂OH.

In certain embodiments R^{A1} is —CH₂CH₂OH.

### Embodiments of R^{A2}

In certain embodiments R^{A2} is —CH₃.

In certain embodiments R^{A2} is —CH₂X.

In certain embodiments R^{A2} is —CHX₂.

In certain embodiments R^{A2} is —CX₃.

In certain embodiments R^{A2} is —CH₂CH₃.

In certain embodiments R^{A2} is —CH₂CH₂X.

In certain embodiments R^{A2} is —CH₂CHX₂.

In certain embodiments R^{A2} is —CH₂CX₃.

In certain embodiments R^{A2} is —CH₂OH.

In certain embodiments R^{A2} is —CH₂CH₂OH.

### Embodiments of R^{A3}

In certain embodiments R^{A3} is —CH₂X.

In certain embodiments R^{A3} is —CHX₂.

In certain embodiments R^{A3} is —CX₃.

In certain embodiments R^{A3} is —CH₂CH₃.

In certain embodiments R^{A3} is —CH₂CH₂X.

In certain embodiments R^{A3} is —CH₂CHX₂.

In certain embodiments R^{A3} is —CH₂CX₃.

In certain embodiments R^{A3} is —CH₂OH.

In certain embodiments R^{A3} is —CH₂CH₂OH.

### Embodiments of R^{A4}

In certain embodiments R^{A4} is hydrogen.

In certain embodiments R^{A4} is —CH₂X.

In certain embodiments R^{A4} is —CHX₂.

In certain embodiments R^{A4} is —CX₃.

In certain embodiments R^{A4} is —CH₂CH₃.

In certain embodiments R^{A4} is —CH₂CH₂X.

In certain embodiments R^{A4} is —CH₂CHX₂.

In certain embodiments R^{A4} is —CH₂CX₃.

In certain embodiments R^{A4} is —CH₂OH.

In certain embodiments R^{A4} is —CH₂CH₂OH.

### Embodiments of R^{B1}

In certain embodiments R^{B1} is

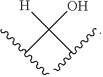 [SMILES: *C(*)O]

In certain embodiments R^{B1} is

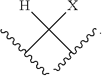

In certain embodiments R^{B1} is

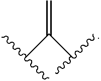 [SMILES: *C(*)=C]

In certain embodiments R^{B1} is

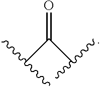 [SMILES: *C(*)=O]

### Embodiments of R^{B2}

In certain embodiments R^{B2} is

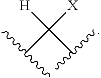

In certain embodiments R^{B2} is

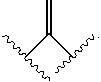 [SMILES: *C(*)=C]

### Embodiments of R^{B3}

In certain embodiments R^{B3} is

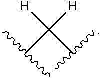 [SMILES: *C*]

In certain embodiments R^{B3} is

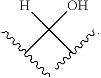 [SMILES: *C(*)O]

In certain embodiments R^{B3} is

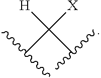

In certain embodiments R^{B3} is

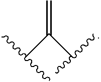 [SMILES: *C(*)=C]

In certain embodiments R^{B3} is

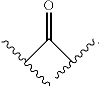 [SMILES: *C(*)=O]

### Embodiments of R^{N1}

In certain embodiments R^{N1} is hydrogen.

In certain embodiments R^{N1} is —(C₁-C₆)alkyl.

In certain embodiments R^{N1} is —CH₂CH₂X.

In certain embodiments R^{N1} is —CH₂CHX₂.

In certain embodiments R^{N1} is —CH₂CX₃.

In certain embodiments R^{N1} is —CH₂CH₂OH.

### Embodiments of R^{N2}

In certain embodiments R^{N2} is —(C₃-C₆)alkyl.

In certain embodiments R^{N2} is —CH₂CH₂X.

In certain embodiments R^{N2} is —CH₂CHX₂.

In certain embodiments R^{N2} is —CH₂CX₃.

In certain embodiments R^{N2} is —CH₂CH₂OH.

In certain embodiments R^{N2} is hydroxy.

### Embodiments of R^{N3}

In certain embodiments R^{N3} is —(C₁-C₆)alkyl.

In certain embodiments R^{N3} is —CH₂CH₂X.

In certain embodiments R^{N3} is —CH₂CHX₂.

In certain embodiments R^{N3} is —CH₂CX₃.

In certain embodiments R^{N3} is —CH₂CH₂OH.

In certain embodiments R^{N3} is and hydroxy.

### Embodiments of R^{N4}

In certain embodiments R^{N4} is —(C₁-C₆)alkyl.

In certain embodiments R^{N4} is —CH₂CH₂X.

In certain embodiments R^{N4} is —CH₂CHX₂.

In certain embodiments R^{N4} is —CH₂CX₃.

In certain embodiments R^{N4} is —CH₂CH₂OH.

### Embodiments of R^{N5}

In certain embodiments R^{N5} is hydrogen.

In certain embodiments R^{N5} is —(C₁-C₆)alkyl.

In certain embodiments R^{N5} is —CH₂CH₂X.

In certain embodiments R^{N5} is —CH₂CHX₂.

In certain embodiments R^{N5} is —CH₂CX₃.

In certain embodiments R^{N5} is —CH₂CH₂OH.

In certain embodiments R^{N5} is hydroxy.

### Embodiments of R^{N6}

In certain embodiments R^{N6} is —(C₂-C₆)alkyl.

In certain embodiments R^{N6} is —CH₂CH₂X.

In certain embodiments R^{N6} is —CH₂CHX₂.

In certain embodiments R^{N6} is —CH₂CX₃.

In certain embodiments R^{N6} is —CH₂CH₂OH.

In certain embodiments R^{N6} is hydroxy.

### Embodiments of R^{N7}

In certain embodiments each R^{N7} is hydrogen.

In certain embodiments each R^{N7} is —(C₂-C₆)alkyl.

In certain embodiments each R^{N7} is —CH₂CH₂X.

In certain embodiments each R^{N7} is —CH₂CHX₂.

In certain embodiments each R^{N7} is —CH₂CX₃.

In certain embodiments each R^{N7} is —CH₂CH₂OH.

In certain embodiments one R^{N7} is and hydroxy.

In certain embodiments one R^{N7} is hydrogen.

In certain embodiments one R^{N7} is —(C₂-C₆)alkyl.

In certain embodiments one R^{N7} is —CH₂CH₂X.

In certain embodiments one R^{N7} is —CH₂CHX₂.

In certain embodiments one R^{N7} is —CH₂CX₃.

In certain embodiments one R^{N7} is —CH₂CH₂OH.

### Embodiments of X

In certain embodiments X is —F.

In certain embodiments X is —Cl.

In certain embodiments X is —Br.

### Preparation of Enantiomeric Compounds

Various methods are known in the art for preparing optically active forms and determining activity. Such methods include standard tests described herein and other similar tests which are well known in the art. Examples of methods that can be used to obtain optical isomers of a compound according to the present disclosure include the following:
i) physical separation of crystals whereby macroscopic crystals of the individual enantiomers are manually separated. This technique may particularly be used if crystals of the separate enantiomers exist (i.e., the material is a conglomerate), and the crystals are visually distinct;
ii) simultaneous crystallization whereby the individual enantiomers are separately crystallized from a solution of the racemate, possible only if the latter is a conglomerate in the solid state;
iii) enzymatic resolutions whereby partial or complete separation of a racemate by virtue of differing rates of reaction for the enantiomers with an enzyme;
iv) enzymatic asymmetric synthesis, a synthetic technique whereby at least one step of the synthesis uses an enzymatic reaction to obtain an enantiomerically pure or enriched synthetic precursor of the desired enantiomer;
v) chemical asymmetric synthesis whereby the desired enantiomer is synthesized from an achiral precursor under conditions that produce asymmetry (i.e., chirality) in the product, which may be achieved using chiral catalysts or chiral auxiliaries;
vi) diastereomer separations whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomers;
viii) kinetic resolutions comprising partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors whereby the desired enantiomer is obtained from non-chiral starting materials and where the stereochemical integrity is not or is only minimally compromised over the course of the synthesis;
x) chiral liquid chromatography whereby the enantiomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography whereby the racemate is volatilized and enantiomers are separated by virtue of their differing interactions in the gaseous mobile phase with a column containing a fixed non-racemic chiral adsorbent phase;
xii) extraction with chiral solvents whereby the enantiomers are separated by virtue of preferential dissolution of one enantiomer into a particular chiral solvent; and
xiii) transport across chiral membranes whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane, which allows only one enantiomer of the racemate to pass through.

### Enantiomerically Enriched Pharmaceutical Compositions

A chiral compound of the invention may be prepared by chiral chromatography from the racemic or stereoisomerically enriched free amine. Pharmaceutically acceptable salts of a chiral compound may be prepared from fractional crystallization of salts from a racemic or an enantiomerically enriched free amine and a chiral acid. Alternatively, the free amine may be reacted with a chiral auxiliary and the enantiomers separated by chromatography followed by removal of the chiral auxiliary to regenerate the free amine. Furthermore, separation of enantiomers may be performed at any convenient point in the synthesis of a compound of the invention. A compound of the invention may also be prepared using a chiral synthesis.

An enantiomerically enriched mixture is a mixture that contains one enantiomer in a greater amount than the other. An enantiomerically enriched mixture of an S-enantiomer contains at least 55% of the S-enantiomer, and more typically at least about 60%, 65%, 70%, 75%, 80%, 85%, 90%, 95% of the S-enantiomer. An enantiomerically enriched mixture of an R-enantiomer contains at least 55% of the R-enantiomer, more typically at least about 55%, 60%, 65%, 70%, 75%, 80%, 85%, 90%, 95% of the R-enantiomer.

Any one of Structures I-XLV or a compound, pure enantiomer or enantiomerically enriched mixture of Formula I, II, or III optionally may be provided in a composition that is enantiomerically enriched, such as a mixture of enantiomers in which one enantiomer is present in excess, in particular to the extent of 60% or more, 70% or more, 75% or more, 80% or more, 90% or more, 95% or more, or 98% or more, including 100%.

### Methods to Treat CNS Disorders Including Mental Disorders and for Mental Enhancement

The present invention also provides methods for modulating the CNS in a human by administering a pharmaceutically effective amount of the compound(s) of the present invention, for example, Structures I or II.

In other aspects the present invention provides a method for modulating the CNS of a non-human domesticated mammal comprising administering a pharmaceutically effective amount of the compound(s) of the present invention, for example, Structures I or II. Non-limiting examples of non-human domesticated mammals include: cat, dog, goat, sheep, horse, and cow.

The present invention additionally provides methods for modulating the CNS in mammals for example a human by administering a pharmaceutically effective amount of the compound(s) of Formula I, Formula II, or Formula III.

Structures I-XLV and a compound, pure enantiomer or enantiomerically enriched mixture of Formula I, II, or III disclosed herein are useful in methods for treating a variety of diseases or disorders linked to inadequate functioning of neurotransmission in the CNS of mammals. Included among such disorders are depression, dysthymia, anxiety and phobia disorders (including generalized anxiety, social anxiety, panic, post-traumatic stress and adjustment disorders), feeding and eating disorders (including binge eating, bulimia, and anorexia nervosa), other binge behaviors, body dysmorphic syndromes, alcoholism, tobacco abuse, drug abuse or dependence disorders, disruptive behavior disorders, impulse control disorders, gaming disorders, gambling disorders, memory loss, dementia of aging, attention deficit hyperactivity disorder, personality disorders (including antisocial, avoidant, borderline, histrionic, narcissistic, obsessive compulsive, paranoid, schizoid and schizotypal personality disorders), attachment disorders, autism, and dissociative disorders.

In addition to treating various diseases and disorders, the employed methods of modulating activity of the serotonergic system in particular can be used to improve CNS functioning in non-disease states, such as reducing neuroticism and psychological defensiveness, increasing openness to experience, increasing creativity, and aiding decision-making. Any of these methods can employ a compound, pure enantiomer or enantiomerically enriched mixture of Formula I, II, or III or any one of Structures I-XLV, either as a racemate, an individual enantiomer, an enantiomerically enriched mixture, or with deuterium-substitution, or more than one of these in combination. When referring to Structures herein, the terms accordingly should be understood to refer not only to the racemates of those structures, but also to single enantiomers, enantiomerically enriched mixtures, and structures with deuterium-substitution(s) or other modifications, as the context indicates and supports.

This invention also provides the use of a compound, pure enantiomer or enantiomerically enriched mixture of Formula I, II, or III, or any of Structures I-XLV for the manufacture of a medicament for the treatment of maladaptive response to perceived psychological threats. Additionally, this invention provides a pharmaceutical formulation adapted for the treatment of maladaptive response to perceived psychological threats containing a compound, pure enantiomer or enantiomerically enriched mixture of Formula I, II, or III, or any one of Structures I-XLV. Furthermore, this invention includes a method for the treatment of maladaptive response to perceived psychological threats that comprises administering an effective amount of a compound, pure enantiomer or enantiomerically enriched mixture of Formula I, II, or III, or any one of Structures I-XLV, given either in the context of psychotherapy or as a stand-alone treatment.

### Methods to Treat Headache Disorders

In certain embodiments, a method of treating a patient with primary or secondary headaches is provided, comprising administering an effective amount of a compound, pure enantiomer, or enantiomerically enriched mixture of Structures I-XLV, or a pharmaceutically acceptable salt thereof.

In other embodiments, a method of treating a patient with primary or secondary headaches is provided, comprising administering an effective amount of a compound, pure enantiomer, or enantiomerically enriched mixture of Formula I, II, or III, or a pharmaceutically acceptable salt thereof.

As used herein, primary headaches include, but are not limited to migraine, migraine signs and symptoms without cephalgia, tension-type headaches, cluster headaches and other trigeminal autonomic cephalalgias, new daily persistent headache, hypnic headaches, stabbing headaches, and other primary headache disorders. Secondary headaches referred to herein can refer to those due to trauma or injury, cranial or cervical vascular disorder, non-vascular intracranial disorder, headaches due to substance use or substance withdrawal, and other secondary headaches.

### Non-Limiting Examples of Pharmacotherapeutic Counseling Use

Psychotherapy, cognitive enhancement, or life coaching conducted with a compound or pharmaceutically acceptable salt as described herein employed as an adjunct (hereafter, “pharmacotherapy” or “pharmacotherapy counseling”) is typically conducted in widely spaced sessions with one, two, or rarely three or more administrations of an entactogen per session. These sessions can be as frequent as weekly but are more often approximately monthly or even less frequently. In most cases, a small number of pharmacotherapy counseling sessions, on the order of one to three, is needed for the patient to experience significant clinical progress, as indicated, for example, by a reduction in signs and symptoms of mental distress, by improvement in functioning in some domain of life, by arrival at a satisfactory solution to some problem, or by increased feelings of closeness to and understanding of some other person. In some embodiments, the psychotherapy, cognitive enhancement, or life coaching is conducted with an effective amount of enantiomerically enriched compound of Formula I, II, or III, or any one of Structures I-XLV or a pharmaceutically acceptable salt thereof.

The following sections provide detailed examples of pharmacotherapy. While common procedures are described, these are intended as illustrative, non-restrictive examples. It is anticipated that the prescribing physician and therapy team may wish to specify different procedures than those described here based on their clinical judgment concerning the needs of the patient.

The example methods of treatment can also be modified with very minor changes to treat multiple patients at once, including couples or families. Hence, “patient” should be understood to mean one or more individuals.

Use of a Compound or Composition of the Present Invention in Conjunction with Conventional Psychotherapy or Coaching

In certain embodiments, the use of a described indolizine compound or composition of the present invention as pharmacotherapy is integrated into the patient's ongoing psychotherapy or coaching (hereafter abbreviated as “psychotherapy”). If a patient in need of the pharmacotherapy is not in ongoing psychotherapy, then psychotherapy may be initiated and the pharmacotherapy counseling added later, after the prescribing physician and treating psychotherapist, physician, coach, member of the clergy, or other similar professional or someone acting under the supervision of such a professional (hereafter, “therapist”) agree that the pharmacotherapy counseling is indicated and that there have been sufficient meetings between the patient and therapist to establish an effective therapeutic alliance.

If the patient is not experienced with the pharmacotherapy, a conversation typically occurs in which the therapist or other members of the therapy team addresses the patient's questions and concerns about the medicine and familiarizes the patient with the logistics of pharmacotherapy-assisted session. The therapist describes the kinds of experience that can be expected during the pharmacotherapy session. Optionally, parts of this conversation employ written, recorded, or interactive digital explanations, as might be used in the informed consent process in a clinical trial. The therapist may additionally make commitments to support the participant's healthcare and wellness process. In turn, the patient may be asked to make commitments of their own (such as not to hurt themselves or others and to abstain from contra-indicated medicines or drugs for an adequate period before and after the pharmacotherapy).

A compound or composition of the invention (or alternately herein for convenience, the “medicine”) is administered shortly before or during a scheduled psychotherapy session, with timing optionally selected so that therapeutic effects begin by the time the psychotherapy session begins. It is to be understood that references to administering the medicine “during” a psychotherapeutic or other session are intended to refer to timing the administration of the medicine such that the therapeutic effects of the medicine at least partly temporally overlap with the therapeutic effects of the session. Either shortly before or after administration of the medicine, it is common for the therapist to provide some reminder of their mutual commitments and expected events during the session.

The psychotherapy session is carried out by the therapist, who, optionally, may be remote and in communication with the patient using a communication means suitable for telehealth or telemedicine, such as a phone, video, or other remote two-way communication method. Optionally, video or other monitoring of the patient's response or behavior is used to document or measure the session. The therapist uses their clinical judgment and available data to adjust the session to the needs of the patient. Many therapists view their responsibility as being to facilitate rather than direct the patient's experience. This may sometimes involve silent empathic listening, while other times it may include more active support to help the patient arrive at new perspectives on their life.

It is anticipated that the therapeutic effects of the medicine will allow the patient to make more rapid therapeutic progress than would normally be possible. These effects include decreased neuroticism and increased feelings of authenticity. Patients are often able to calmly contemplate actual or possible experiences that would normally be upsetting or even overwhelming. This can facilitate decision making and creativity in addition to mental wellness.

Optionally, the prescribing physician may allow a second or even third administration of the medicine or another psychotherapeutic agent in order to extend the therapeutic effects. Optionally, a pharmaceutical preparation with modified release is employed to make this unnecessary.

Because the duration of the scheduled psychotherapy session may be shorter than the therapeutic effects of the medicine, the therapist may suggest to the patient activities to support further psychotherapeutic progress after the psychotherapy session has ended. Alternatively, the therapist may continue to work with the patient until the therapeutic effects of the medicine have become clinically minimal.

In a subsequent non-pharmacological psychotherapy session, the therapist and patient will typically discuss the patient's experiences from the pharmacotherapy session and the therapist will often aid the patient in recalling the therapeutic effects and help them to incorporate the experiences into their everyday lives.

Pharmacotherapy sessions may be repeated as needed, based on the judgment of the treating physician and therapy team regarding the needs of the patient.

### Use of a Compound or Composition of the Present Invention Outside of Conventional Psychotherapy

In certain embodiments, a compound or composition of the present invention is administered outside of a conventional psychotherapy. This method is a broader, more flexible approach to pharmacotherapy that is not centered on supervision by a therapist. These pharmacotherapy sessions can take place in many different quiet and safe settings, including the patient's home. The setting is typically chosen to offer a quiet setting, with minimal disruptions, where the patient feels psychologically safe and emotionally relaxed. The setting may be the patient's home but may alternatively be a clinic, retreat center, or hotel room.

Optionally, a checklist may be followed to prepare the immediate environment to minimize distractions and maximize therapeutic or decision-making benefits. This checklist can include items such as silencing phones and other communications devices, cleaning and tidying the environment, preparing light refreshments, preparing playlists of appropriate music, and pre-arranging end-of-session transportation if the patient is not undergoing pharmacotherapy at home.

Before the pharmacotherapy session, there may be an initial determination of the therapeutic or other life-related goals (for example, decision-making, increasing creativity, or simply appreciation of life) that will be a focus of the session. These goals can optionally be determined in advance with support from a therapist.

Optionally, the therapist may help the patient select stimuli, such as photographs, videos, augmented or virtual reality scenes, or small objects such as personal possessions, that will help focus the patient's attention on the goals of the session or on the patient's broader life journey. As examples that are intended to be illustrative and not restrictive, these stimuli can include photographs of the patient from when they were young, which can increase self-compassion, or can include stimuli relating to traumatic events or phobias experienced by the patient, which can help the patient reevaluate and change their response to such stimuli. Optionally, the patient selects these stimuli without assistance (for example, without the involvement of the therapist) or does not employ any stimuli. Optionally, stimuli are selected in real time by the therapist or an algorithm based on the events of the session with the goal of maximizing benefits to the patient.

If the patient is not experienced with the pharmacotherapy, a conversation occurs in which the therapist addresses the patient's questions and concerns about the medicine and familiarizes the patient with the logistics of a pharmacotherapy-assisted session. The therapist describes the kinds of experience that can be expected during the pharmacotherapy-assisted session. Optionally, parts of this conversation employ written, recorded, or interactive digital explanations, as might be used in the informed consent process in a clinical trial. The therapist may additionally make commitments to support the participant's healthcare and wellness process. In turn, the patient may be asked to make commitments of their own (such as not to hurt themselves or others and to abstain from contraindicated medicines or drugs for an adequate period before and after the pharmacotherapy).

Selected session goals and any commitments or other agreements regarding conduct between the patient and therapy team are reviewed immediately before administration of the medicine. Depending on the pharmaceutical preparation and route of administration, the therapeutic effects of the medicine usually begin within one hour. Typical therapeutic effects include decreased neuroticism and increased feelings of authenticity. Patients are often able to calmly contemplate experiences or possible experiences that would normally be upsetting or even overwhelming. This can facilitate decision making and creativity in addition to mental wellness.

Optionally, sleep shades and earphones with music or soothing noise may be used to reduce distractions from the environment. Optionally, a virtual reality or immersive reality system may be used to provide stimuli that support the therapeutic process. Optionally, these stimuli are preselected; optionally, they are selected in real time by a person, or an algorithm based on events in the session with the goal of maximizing benefits to the patient. Optionally, a therapist or other person well-known to the patient is present or available nearby or via phone, video, or other communication method in case the patient wishes to talk, however the patient may optionally undergo a session without the assistance of a therapist. Optionally, the patient may write or create artwork relevant to the selected session goals. Optionally, the patient may practice stretches or other beneficial body movements, such as yoga (“movement activity”).

Optionally, in other embodiments the patient may practice movement activity that includes more vigorous body movements, such as dance or other aerobic activity. Movement activity also may make use of exercise equipment such as a treadmill or bicycle.

In some additional embodiments, the patient may be presented with music, video, auditory messages, or other perceptual stimuli. Optionally, these stimuli may be adjusted based on the movements or other measurable aspects of the patient. Such adjustment may be done by the therapist with or without the aid of a computer, or by a computer alone in response to the patient aspects, including by an algorithm or artificial intelligence, and “computer” broadly meaning any electronic tool suitable for such purposes, whether worn or attached to a patient (for example, watches, fitness trackers, “wearables,” and other personal devices; biosensors or medical sensors; medical devices), whether directly coupled or wired to a patient or wirelessly connected (and including desktop, laptop, and notebook computers; tablets, smartphones, and other mobile devices; and the like), and whether within the therapy room or remote (for example, cloud-based systems).

For example, measurable aspects of a patient (for example, facial expression, eye movements, respiration rate, pulse rate, skin color change, patient voice quality or content, patient responses to questions) from these tools may be individually transformed into scores on standardized scales by subtracting a typical value and then multiplying by a constant and these scores may be further multiplied by constants and added together to create an overall score that can optionally be transformed by multiplication with a link function, such as the logit function, to create an overall score. This score may be used to select or adjust stimuli such as selecting music with higher or lower beats-per-minute or with faster or slower notes, selecting images, audio, or videos with different emotionality or autobiographical meaning, or selecting activities for the patient to engage in (such as specific movements, journaling prompts, or meditation mantras).

It should be readily appreciated that a patient can participate in numerous therapeutically beneficial activities, where such participation follows or is in conjunction with the administration of a compound or composition of the invention, including writing about a preselected topic, engaging in yoga or other movement activity, meditating, creating art, viewing of photographs or videos or emotionally evocative objects, using a virtual reality or augmented reality system, talking with a person, and thinking about a preselected problem or topic, and it should be understood that such participation can occur with or without the participation or guidance of a therapist.

Optionally, the prescribing physician may allow a second or even third administration of the medicine or another psychotherapeutic agent in order to extend the therapeutic effects. Optionally, a pharmaceutical preparation with modified release is employed to make this unnecessary.

The patient typically remains in the immediate environment until the acute therapeutic effects of the medicine are clinically minimal, usually within eight hours. After this point, the session is considered finished.

The treatment plan will often include a follow-up session with a therapist. This follow-up session occurs after the pharmacotherapy counseling session has ended, often the next day but sometimes several days later. In this session, the patient discusses their experiences from the pharmacotherapy counseling session with the therapist, who can aid them in recalling the therapeutic effects and help them to incorporate the experiences into their everyday lives.

Pharmacotherapy counseling sessions may be repeated as needed, based on the judgment of the treating physician and therapy team regarding the needs of the patient.

### Pharmaceutical Compositions and Salts

While it is possible to administer a compound employed in the methods of this invention directly without any formulation, a compound is usually administered in the form of pharmaceutical compositions comprising a pharmaceutically acceptable carrier, diluent, or excipient, and at least one active ingredient. “Pharmaceutically acceptable” as used in connection with an excipient, carrier, or diluent means an excipient, carrier, or diluent that is useful in preparing a pharmaceutical composition that is generally safe, non-toxic, and neither biologically nor otherwise undesirable for veterinary use and/or human pharmaceutical use. These compositions can be administered by a variety of routes including systemic, topical, parenteral, oral, mucosal (for example, buccal, sublingual), rectal, transdermal, subcutaneous, intravenous, intramuscular, inhaled, and intranasal. Such compositions are prepared in a manner well known in the pharmaceutical art and comprise at least one active compound. (See, for example, Remington, 2005, Remington: The science and practice of pharmacy, 21st ed., Lippincott Williams & Wilkins.)

The pharmaceutical composition may be formulated as any pharmaceutically useful form, for example, a solid dosage form, a liquid, an aerosol, a cream, a gel, a pill, an injection or infusion solution, a capsule, a tablet, a syrup, a transdermal patch, a subcutaneous patch, a dry powder, an inhalation formulation, a suppository, a buccal or sublingual formulation, a parenteral formulation, an ophthalmic solution, or in a medical device. Some dosage forms, such as tablets and capsules, are subdivided into suitably sized unit doses containing appropriate quantities of the active components, for example, an effective amount to achieve the desired purpose.

A “pharmaceutically acceptable composition” thus refers to at least one compound (which may be a mixture of enantiomers or diastereomers, as fully described herein) of the invention and a pharmaceutically acceptable vehicle, excipient, diluent or other carrier in an effective amount to treat a host, typically a human, who may be a patient.

In certain nonlimiting embodiments the pharmaceutical composition is a dosage form that contains from about 0.1 mg to about 1500 mg, from about 10 mg to about 1000 mg, from about 100 mg to about 800 mg, or from about 200 mg to about 600 mg of the active compound and optionally from about 0.1 mg to about 1500 mg, from about 10 mg to about 1000 mg, from about 100 mg to about 800 mg, or from about 200 mg to about 600 mg of an additional active agent in a unit dosage form. Examples are dosage forms with at least 0.1, 1, 5, 10, 20, 25, 40, 50, 100, 125, 150, 200, 250, 300, 400, 500, 600, 700, or 750 mg of active compound, or its salt or salt mixture.

In certain nonlimiting embodiments the pharmaceutical composition is a dosage form that contains at least about 0.1 mg, at least about 10 mg, at least about 100 mg, at least about 200 mg, no more than about 1000 mg, not more than about 800 mg, or nor more than about 600 mg of an additional active agent in a unit dosage form. Examples are dosage forms with about 0.1, 1, 5, 10, 20, 25, 40, 50, 100, 125, 150, 200, 250, 300, 400, 500, 600, 700, or 750 mg of active compound, or its salt or salt mixture.

The pharmaceutical compositions described herein can be formulated into any suitable dosage form, including aqueous oral dispersions, aqueous oral suspensions, solid dosage forms including oral solid dosage forms, aerosols, controlled release formulations, fast melt formulations, effervescent formulations, self-emulsifying dispersions, solid solutions, liposomal dispersions, lyophilized formulations, tablets, capsules, pills, powders, delayed-release formulations, immediate-release formulations, modified release formulations, extended-release formulations, pulsatile release formulations, multi particulate formulations, and mixed immediate release and controlled release formulations. Generally speaking, one will desire to administer an amount of the active agents of the present invention that is effective to achieve a plasma level commensurate with the concentrations found to be effective in vivo for a period of time effective to elicit a desired therapeutic effect without abuse liability.

In making the compositions employed in the present invention the active ingredient is usually mixed with an excipient, diluted by an excipient, or enclosed within such a carrier which can be in the form of a capsule, sachet, paper or other container. When the excipient serves as a diluent, it can be a solid, semi-solid, or liquid material, which acts as a vehicle, carrier, or medium for the active ingredient. Thus, the compositions can be in the form of tablets (including orally disintegrating, swallowable, sublingual, buccal, and chewable tablets), pills, powders, lozenges, troches, oral films, thin strips, sachets, cachets, elixirs, suspensions, emulsions, solutions, slurries, syrups, aerosols (as a solid or in a liquid medium), ointments containing for example up to 10% by weight of the active compound, soft and hard gelatin capsules, suppositories, dry powders for inhalation, liquid preparations for vaporization and inhalation, topical preparations, transdermal patches, sterile injectable solutions, and sterile packaged powders. Compositions may be formulated as immediate release, controlled release, sustained (extended) release or modified release formulations.

Other embodiments of the invention include multiple routes of administration, which may differ in different patients according to their preference, co-morbidities, side effect profile, and other factors (IV, PO, transdermal, etc.). Other embodiments of the invention include the presence of other substances with the active drugs, known to those skilled in the art, such as fillers, carriers, gels, skin patches, lozenges, or other modifications in the preparation to facilitate absorption through various routes (such as gastrointestinal, transdermal, etc.) and/or to extend the effect of the drugs, and/or to attain higher or more stable serum levels or to enhance the therapeutic effect of the active drugs in the combination.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, for example, about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxybenzoates; sweetening agents; and flavoring agents. The compositions of the invention can be formulated so as to provide quick, sustained or delayed release of the active ingredient after administration to the patient by employing procedures known in the art.

The compositions in certain non-limiting embodiments formulated in a unit dosage form, each dosage containing from about 0.05 to about 350 mg, more typically about 1.0 to about 180 mg, of the active ingredients. The term “unit dosage form” refers to physically discrete units suitable as unitary dosages for human subjects and other mammals, each unit containing a predetermined quantity of active material calculated to produce the desired therapeutic effect, in association with a suitable pharmaceutical carrier, diluent, or excipient.

For example, some dosages fall within the range of at least about 0.007 to about 4 mg/kg or less. In the treatment of adult humans, the range of at least about 0.1 to about 3 mg/kg or less, in single dose may be useful.

It will be understood that the amount of the compound actually administered will be determined by a physician, in light of the relevant circumstances, including the condition to be treated, the chosen route of administration, the actual compound or compounds administered, the age, weight, and response of the individual patient, and the severity of the patient's symptoms, and therefore the above dosage ranges are not intended to limit the scope of the invention in any way.

In some instances, dosage levels below the lower limit of the aforesaid range may be more than adequate, while in other cases still larger doses may be employed without causing any harmful side effects, provided for instance that such larger doses may be first divided into several smaller doses for administration.

Generally, the pharmaceutical compositions of the invention may be administered and dosed in accordance with good medical practice, taking into account the method and scheduling of administration, prior and concomitant medications and medical supplements, the clinical condition of the individual patient and the severity of the underlying disease, the patient's age, sex, body weight, and other such factors relevant to medical practitioners, and knowledge of the particular compound(s) used. Starting and maintenance dosage levels thus may differ from patient to patient, for individual patients across time, and for different pharmaceutical compositions, but shall be able to be determined with ordinary skill.

In other embodiments, a powder comprising the active agents of the present invention formulations described herein may be formulated to comprise one or more pharmaceutical excipients and flavors. Such a powder may be prepared, for example, by mixing the active agents of the present invention formulation and optional pharmaceutical excipients to form a bulk blend composition. Additional embodiments also comprise a suspending agent and/or a wetting agent. This bulk blend is uniformly subdivided into unit dosage packaging or multi-dosage packaging units. The term “uniform” means the homogeneity of the bulk blend is substantially maintained during the packaging process.

### Oral Formulations

In certain embodiments, a compound, pure enantiomer or enantiomerically enriched mixture of Formula I, II, or III, or any one of Structures I-XLV may be formulated in a pharmaceutically acceptable oral dosage form. Oral dosage forms may include but are not limited to, oral solid dosage forms and oral liquid dosage forms. Oral solid dosage forms may include but are not limited to, tablets, capsules, caplets, powders, pellets, multiparticulates, beads, spheres and/or any combinations thereof. These oral solid dosage forms may be formulated as immediate release, controlled release, sustained (extended) release or modified release formulations.

The oral solid dosage forms of the present invention may also contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof.

In some embodiments, the solid dosage forms of the present invention may be in the form of a tablet (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, for example, capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including a fast-melt tablet. Additionally, pharmaceutical formulations of the present invention may be administered as a single capsule or in multiple capsule dosage form. In some embodiments, the pharmaceutical formulation is administered in two, or three, or four, capsules or tablets.

The pharmaceutical solid dosage forms described herein can comprise the active agents of the present invention compositions described herein and one or more pharmaceutically acceptable additives such as a compatible carrier, binder, complexing agent, ionic dispersion modulator, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

In still other aspects, using standard coating procedures, such as those described in Remington's Pharmaceutical Sciences, 20th Edition (2000), a film coating is provided around the active agent of the present invention formulation. In certain embodiments, some or all of the active agent of the present invention particles are coated. In another embodiment, some or all of the active agent of the present invention particles are microencapsulated. In yet another embodiment, some or all of the active agent of the present invention is amorphous material coated and/or microencapsulated with inert excipients. In still another embodiment, the active agent of the present invention particles are not microencapsulated and are uncoated.

Suitable carriers for use in the solid dosage forms described herein include acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include lactose, calcium carbonate, calcium phosphate, dibasic calcium phosphate, calcium sulfate, microcrystalline cellulose (for example, Avicel®, Avicel® PH101, Avicel® PH102, Avicel® PH105, etc.), cellulose powder, dextrose, dextrates, dextrose, dextran, starches, pregelatinized starch, hydroxypropylmethylcellulose (HPMC), hydroxypropylmethylcellulose phthalate, hydroxypropylmethylcellulose acetate stearate (HPMCAS), sucrose, xylitol, lactitol, mannitol, sorbitol, sodium chloride, polyethylene glycol, and the like.

If needed, suitable disintegrants for use in the solid dosage forms described herein include natural starch such as corn starch or potato starch, a pregelatinized starch such as National 1551 or Amijel®, or a sodium starch glycolate such as Promogel® or Explotab®, a cellulose such as a wood product, microcrystalline cellulose, for example, Avicel®, Avicel® PH101, Avicel® PH102, Avicel® PH105, Elcema® P100, Emcocel®, Vivacel®, Ming Tia®, and Solka-Floc®, Ac-Di-Sol, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder-filled capsule formulation, they aid in plug formation that can be filled into soft- or hard-shell capsules and in tablet formulation, binders ensure that the tablet remains intact after compression and help assure blend uniformity prior to a compression or fill step. Materials suitable for use as binders in the solid dosage forms described herein include carboxymethylcellulose, methylcellulose (for example, Methocel®), hydroxypropylmethylcellulose (for example, Hypromellose USP Pharmacoat-603, hydroxypropylmethylcellulose acetate stearate (Agoate HS-LF and HS), hydroxyethylcellulose, hydroxypropylcellulose (for example, Klucel®), ethylcellulose (for example, Ethocel®), and microcrystalline cellulose (for example, Avicel®), microcrystalline dextrose, amylose, magnesium aluminum silicate, polysaccharide acids, bentonites, gelatin, polyvinylpyrrolidone/vinyl acetate copolymer, crosspovidone, povidone, starch, pregelatinized starch, tragacanth, dextrin, a sugar, such as sucrose (for example, Dipac®), glucose, dextrose, molasses, mannitol, sorbitol, xylitol (for example, Xylitab®), lactose, a natural or synthetic gum such as acacia, tragacanth, ghatti gum, mucilage of isapol husks, starch, polyvinylpyrrolidone (for example, Povidone® CL, Kollidon® CL, Polyplasdone® XL-10, and Povidone® K-12), larch arabogalactan, Veegum®, polyethylene glycol, waxes, sodium alginate, and the like. In general, binder levels of 20-70% are typically used in powder-filled gelatin capsule formulations. Binder usage level in tablet formulations is a function of whether direct compression, wet granulation, roller compaction, or usage of other excipients such as fillers which itself can act as moderate binders are used. Formulators skilled in the art can determine the binder level for the formulations, but binder usage level of up to 70% in tablet formulations is common.

Suitable lubricants or glidants for use in the solid dosage forms described herein include stearic acid, calcium hydroxide, talc, corn starch, sodium stearyl fumarate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include sugars (including lactose, sucrose, and dextrose), polysaccharides (including dextrates and maltodextrin), polyols (including mannitol, xylitol, and sorbitol), cyclodextrins and the like.

Non-water-soluble diluents are compounds typically used in the formulation of pharmaceuticals, such as calcium phosphate, calcium sulfate, starches, modified starches and microcrystalline cellulose, and micro cellulose (for example, having a density of about 0.45 g/cm3, for example Avicel®, powdered cellulose), and talc.

Suitable wetting agents for use in the solid dosage forms described herein include oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (for example, Polyquat 10®), sodium oleate, sodium lauryl sulfate, magnesium stearate, sodium docusate, triacetin, vitamin E TPGS and the like. Wetting agents include surfactants.

Suitable surfactants for use in the solid dosage forms described herein include docusate and its pharmaceutically acceptable salts, sodium lauryl sulfate, sorbitan monooleate, polyoxyethylene sorbitan monooleate, polysorbates, poloxamers, bile salts, glyceryl monostearate, copolymers of ethylene oxide and propylene oxide, for example, Pluronic® (BASF), and the like.

Suitable suspending agents for use in the solid dosage forms described here include polyvinylpyrrolidone, for example, polyvinylpyrrolidone K12, polyvinylpyrrolidone K17, polyvinylpyrrolidone K25, or polyvinylpyrrolidone K30, polyethylene glycol, for example, the polyethylene glycol can have a molecular weight of about 300 to about 6000, or about 3350 to about 4000, or about 7000 to about 18000, vinylpyrrolidone/vinyl acetate copolymer (S630), sodium alginate, gums, such as, for example, gum tragacanth and gum acacia, guar gum, xanthans, including xanthan gum, sugars, cellulosic, such as, for example, sodium carboxymethylcellulose, methylcellulose, sodium carboxymethylcellulose, hydroxypropylmethylcellulose, hydroxyethylcellulose, polysorbate-80, polyethoxylated sorbitan monolaurate, polyethoxylated sorbitan monolaurate, povidone and the like.

Suitable antioxidants for use in the solid dosage forms described herein include, for example, butylated hydroxytoluene (BHT), butyl hydroxyanisole (BHA), sodium ascorbate, Vitamin E TPGS, ascorbic acid, sorbic acid and tocopherol.

Immediate-release formulations may be prepared by combining superdisintegrants such as Croscarmellose sodium and different grades of microcrystalline cellulose in different ratios. To aid disintegration, sodium starch glycolate will be added.

The above-listed additives should be taken as merely examples and not limiting, of the types of additives that can be included in solid dosage forms of the present invention. The amounts of such additives can be readily determined by one skilled in the art, according to the particular properties desired.

Oral liquid dosage forms include solutions, emulsions, suspensions, and syrups. These oral liquid dosage forms may be formulated with any pharmaceutically acceptable excipient known to those of skill in the art for the preparation of liquid dosage forms. For example, water, glycerin, simple syrup, alcohol, and combinations thereof.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as but not limited to, an oil, water, an alcohol, and combinations of these pharmaceutically suitable surfactants, suspending agents, emulsifying agents, may be added for oral or parenteral administration. Suspensions may include oils. Such oils include peanut oil, sesame oil, cottonseed oil, corn oil, and olive oil. Suspension preparation may also contain esters of fatty acids such as ethyl oleate, isopropyl myristate, fatty acid glycerides, and acetylated fatty acid glycerides. Suspension formulations may include alcohols, such as ethanol, isopropyl alcohol, hexadecyl alcohol, glycerol, and propylene glycol. Ethers, such as poly(ethylene glycol), petroleum hydrocarbons such as mineral oil and petrolatum, and water may also be used in suspension formulations.

In some embodiments, formulations are provided comprising particles of a compound, pure enantiomer or enantiomerically enriched mixture of Formula I, II, or III, or any one of Structures I-XLV and at least one dispersing agent or suspending agent for oral administration to a subject. The formulation may be a powder and/or granules for suspension, and upon admixture with water, a substantially uniform suspension is obtained. As described herein, the aqueous dispersion can comprise amorphous and non-amorphous particles consisting of multiple effective particle sizes such that the drug is absorbed in a controlled manner over time. In certain embodiments, the aqueous dispersion or suspension is an immediate-release formulation. In another embodiment, an aqueous dispersion comprising amorphous particles is formulated such that a portion of the particles of the present invention are absorbed within, for example, about 0.75 hours after administration and the remaining particles are absorbed 2 to 4 hours after absorption of the earlier particles.

In other embodiments, addition of a complexing agent to the aqueous dispersion results in a larger span of the particles to extend the drug absorption phase of the active agents such that 50-80% of the particles are absorbed in the first hour and about 90% are absorbed by about 4 hours. Dosage forms for oral administration can be aqueous suspensions selected from the group including pharmaceutically acceptable aqueous oral dispersions, emulsions, solutions, and syrups. See, for example, Singh et al., Encyclopedia of Pharm. Tech., 2nd Ed., 754-757 (2002). In addition to the active agents of the present invention particles, the liquid dosage forms may comprise additives, such as (a) disintegrating agents; (b) dispersing agents; (c) wetting agents; (d) at least one preservative; (e) viscosity enhancing agents; (f) at least one sweetening agent; and (g) at least one flavoring agent.

Examples of disintegrating agents for use in the aqueous suspensions and dispersions include a starch, for example, a natural starch such as corn starch or potato starch, a pregelatinized starch such as National 1551 or Amijel®, or sodium starch glycolate such as Promogel® or Explotab®; a cellulose such as a wood product, microcrystalline cellulose, for example, Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crosspovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropylcellulose ethers (for example, HPC, HPC-SL, and HPC-L), hydroxypropylmethylcellulose and hydroxypropylmethylcellulose ethers (for example, HPMC K100, IPMC K4M, IPMC K15M, and IPMC K100M), carboxymethylcellulose sodium, methylcellulose, hydroxyethylcellulose, hydroxypropylmethylcellulose phthalate, hydroxypropylmethylcellulose acetate stearate, noncrystalline cellulose, magnesium aluminum silicate, triethanolamine, polyvinyl alcohol (PVA), polyvinylpyrrolidone/vinyl acetate copolymer (Plasdone®, for example, S-630), 4-(1,1,3,3-tetramethylbutyl)-phenol polymer with ethylene oxide and formaldehyde (also known as tyloxapol), poloxamers (for example, Pluronics F68®, F88®, and F108®, which are block copolymers of ethylene oxide and propylene oxide); and poloxamines (for example, Tetronic 908®, also known as Poloxamine 908®, which is a tetrafunctional block copolymer derived from sequential addition of propylene oxide and ethylene oxide to ethylenediamine (BASF Corp., Parsippany, N.J.)).

In other embodiments, the dispersing agent is selected from a group not comprising one of the following agents: hydrophilic polymers; electrolytes; Tween® 60 or 80; PEG; polyvinylpyrrolidone (PVP); hydroxypropyl cellulose and hydroxypropyl cellulose ethers (for example, HPC, HPC-SL, and HPC-L); hydroxypropyl methylcellulose and hydroxypropyl methylcellulose ethers (for example, HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethylcellulose phthalate; hydroxypropylmethylcellulose acetate stearate; non-crystalline cellulose; magnesium aluminum silicate; triethanolamine; polyvinyl alcohol (PVA); 4-(1,1,3,3-tetramethyl butyl)-phenol polymer with ethylene oxide and formaldehyde; poloxamers (for example, Pluronics F68®, F88®, and F108®, which are block copolymers of ethylene oxide and propylene oxide); or poloxamines (for example, Tetronic 908® or Poloxamine 908®).

Wetting agents (including surfactants) suitable for the aqueous suspensions and dispersions described herein are known in the art and include acetyl alcohol, glycerol monostearate, polyoxyethylene sorbitan fatty acid esters (for example, the commercially available Tweens® such as for example, Tween 20® and Tween 80® (ICI Specialty Chemicals)), and polyethylene glycols (for example, Carbowaxs 3350® and 1450®, and Carpool 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphatidylcholine and the like.

Suitable preservatives for the aqueous suspensions or dispersions described herein include potassium sorbate, parabens (for example, methylparaben and propylparaben) and their salts, benzoic acid and its salts, other esters of para hydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

In some embodiments, the aqueous liquid dispersion can comprise methylparaben and propylparaben in a concentration ranging from about 0.01% to about 0.3% methylparaben by weight to the weight of the aqueous dispersion and about 0.005% to about 0.03% propylparaben by weight to the total aqueous dispersion weight. In yet another embodiment, the aqueous liquid dispersion can comprise methylparaben from about 0.05 to about 0.1 weight % and propylparaben from about 0.01 to about 0.02 weight % of the aqueous dispersion.

Suitable viscosity enhancing agents for the aqueous suspensions or dispersions described herein include methyl cellulose, xanthan gum, carboxymethylcellulose, hydroxypropyl cellulose, hydroxypropylmethyl cellulose, Plasdone® S-630, carbomer, polyvinyl alcohol, alginates, acacia, chitosans and combinations thereof. The concentration of the viscosity-enhancing agent will depend upon the agent selected and the viscosity desired.

In addition to the additives listed above, the liquid active agents of the present invention formulations can also comprise inert diluents commonly used in the art, such as water or other solvents, solubilizing agents, emulsifiers, and/or sweeteners.

In still other embodiments, effervescent powders containing a compound, pure enantiomer or enantiomerically enriched mixture of Formula I, II, or III, or any one of Structures I-XLV. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the present invention are added to water, the acids and the base react to liberate carbon dioxide gas, thereby causing “effervescence.” Examples of effervescent salts include sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

Tablets of the invention described here can be prepared by methods well known in the art. Various methods for the preparation of the immediate release, modified release, controlled release, and extended-release dosage forms (for example, as matrix tablets, tablets having one or more modified, controlled, or extended-release layers, etc.) and the vehicles therein are well known in the art. Generally recognized compendia of methods include: Remington: The Science and Practice of Pharmacy, Alfonso R. Gennaro, Editor, 20th Edition, Lippincott Williams & Wilkins, Philadelphia, PA; and Sheth et al. (1980), Compressed tablets, in Pharmaceutical dosage forms, Vol. 1, edited by Lieberman and Lachtman, Dekker, NY.

In certain embodiments, solid dosage forms, for example, tablets, effervescent tablets, and capsules, are prepared by mixing the active agents of the present invention particles with one or more pharmaceutical excipients to form a bulk blend composition. When referring to these bulk blend compositions as homogeneous, it is meant that the active agents of the present invention particles are dispersed evenly throughout the composition so that the composition may be readily subdivided into equally effective unit dosage forms, such as tablets, pills, and capsules. The individual unit dosages may also comprise film coatings, which disintegrate upon oral ingestion or upon contact with diluents. These the active agents of the present invention formulations can be manufactured by conventional pharmaceutical techniques.

Conventional pharmaceutical techniques for preparation of solid dosage forms include, for example, one or a combination of methods: (1) dry mixing, (2) direct compression, (3) milling, (4) dry or non-aqueous granulation, (5) wet granulation, or (6) fusion. See, for example, Lachman et al., Theory and Practice of Industrial Pharmacy (1986). Other methods include, for example, spray drying, pan coating, melt granulation, granulation, fluidized bed spray drying or coating (for example, Wurster coating), tangential coating, top spraying, tableting, extruding and the like.

Compressed tablets are solid dosage forms prepared by compacting the bulk blend the active agents of the present invention formulations described above. In various embodiments, compressed tablets which are designed to dissolve in the mouth will comprise one or more flavoring agents. In other embodiments, the compressed tablets will comprise a film surrounding a final compressed tablet. In some embodiments, the film coating can provide a delayed release of the active agents of the present invention formulation. In other embodiments, the film coating aids in patient compliance (for example, Opadry® coatings or sugar coating). Film coatings comprising Opadry® typically range from about 1% to about 3% of the tablet weight. Film coatings for delayed-release usually comprise 2-6% of a tablet weight or 7-15% of a spray-layered bead weight. In other embodiments, the compressed tablets comprise one or more excipients.

A capsule may be prepared, for example, by placing the bulk blend the active agents of the present invention formulation, described above, inside of a capsule. In some embodiments, the active agents of the present invention formulations (non-aqueous suspensions and solutions) are placed in a soft gelatin capsule. In other embodiments, the active agents of the present invention formulations are placed in standard gelatin capsules or non-gelatin capsules such as capsules comprising HPMC. In other embodiments, the active agents of the present invention formulations are placed in a sprinkle capsule, wherein the capsule may be swallowed whole or the capsule may be opened and the contents sprinkled on food prior to eating. In some embodiments of the present invention, the therapeutic dose is split into multiple (for example, two, three, or four) capsules. In some embodiments, the entire dose of the active agents of the present invention formulation is delivered in a capsule form.

In certain embodiments, ingredients (including or not including the active agents) of the invention are wet granulated. The individual steps in the wet granulation process of tablet preparation include milling and sieving of the ingredients, dry powder mixing, wet massing, granulation, drying, and final grinding. In various embodiments, the active agents of the present invention composition are added to the other excipients of the pharmaceutical formulation after they have been wet granulated. Alternatively, the ingredients may be subjected to dry granulation, for example, via compressing a powder mixture into a rough tablet or “slug” on a heavy-duty rotary tablet press. The slugs are then broken up into granular particles by a grinding operation, usually by passage through an oscillation granulator. The individual steps include mixing of the powders, compressing (slugging) and grinding (slug reduction or granulation). No wet binder or moisture is involved in any of the steps.

In some embodiments, the active agents of the present invention formulation are dry granulated with other excipients in the pharmaceutical formulation. In other embodiments, the active agents of the present invention formulation are added to other excipients of the pharmaceutical formulation after they have been dry granulated.

In other embodiments, the formulation of the present invention formulations described herein is a solid dispersion. Methods of producing such solid dispersions are known in the art and include U.S. Pat. Nos. 4,343,789, 5,340,591, 5,456,923, 5,700,485, 5,723,269, and U.S. Pub. No. 2004/0013734. In some embodiments, the solid dispersions of the invention comprise both amorphous and non-amorphous active agents of the present invention and can have enhanced bioavailability as compared to conventional active agents of the present invention formulations. In still other embodiments, the active agents of the present invention formulations described herein are solid solutions. Solid solutions incorporate a substance together with the active agents and other excipients such that heating the mixture results in the dissolution of the drug and the resulting composition is then cooled to provide a solid blend that can be further formulated or directly added to a capsule or compressed into a tablet.

### Non-Limiting Examples of Formulations for Oral Delivery

The examples below provide non-limiting embodiments of formulations for oral delivery, which can be used to deliver any of a compound described herein in enantiomerically enriched form, pure form or even a racemic mixture. Therefore, while the compounds below are specified, any desired purity form or compound can be used if it achieves the desired goal of treatment.

In one non-limiting embodiment, hard gelatin capsules comprising the following ingredients are prepared by mixing the ingredients and filling into hard gelatin capsules in 340 mg quantities.

Hard gelatin capsules containing the following ingredients are prepared:

| Ingredient | Quantity (mg/capsule) |
|---|---|
| Structure II (R-enantiomer or S- | 30.0 |
| enantiomer) | |
| Starch | 205.0 |
| Alpha lipoic acid | 100.0 |
| Magnesium stearate | 5.0 |

The above ingredients are mixed and filled into hard gelatin capsules in 340 mg quantities.

A tablet formula is prepared using the ingredients below:

| Ingredient | Quantity (mg/tablet) |
|---|---|
| Structure II (70% R-enantiomer, 30% S- | 25.0 |
| enantiomer; or 70% S-enantiomer, 30% R | |
| enantiomer) | |
| Cellulose, microcrystalline | 200.0 |
| Colloidal silicon dioxide | 10.0 |
| Stearic acid | 5.0 |

The components are blended and compressed to form tablets, each weighing 240 mg.

Tablets, each containing 30 mg of active ingredient, are prepared as follows:

| Ingredient | Quantity (mg/tablet) |
|---|---|
| Structure I (R-enantiomer or S- | 20.0 |
| enantiomer) | |
| Structure II (Racemic) | 10.0 |
| Starch | 45.0 |
| Microcrystalline cellulose | 35.0 |
| Polyvinylpyrrolidone (as 10% solution in | 4.0 |
| water) | |
| Sodium carboxymethyl starch | 4.5 |
| Magnesium stearate | 0.5 |
| Talc | 1.0 |

The active ingredients, starch and cellulose are passed through a No. 20 mesh U.S. sieve an mixed thoroughly. The solution of polyvinylpyrrolidone is mixed with the resultant powders, which are then passed through a 16 mesh U.S. sieve. The granules so produced are dried at 50-60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

Capsules, each containing 40 mg of active ingredients are made as follows:

| Ingredient | Quantity (mg/capsule) |
|---|---|
| Structure I (racemic) | 10.0 |
| Structure II (R-enantiomer or S- | 30.0 |
| enantiomer) | |
| Starch | 109.0 |
| Magnesium stearate | 1.0 |

The active ingredients, cellulose, starch, and magnesium stearate are blended, passed through a No. 20 mesh U.S. sieve, and filled into hard gelatin capsules in 150 mg quantities.

Capsules, each containing 100 mg of active ingredient, are made as follows:

| Ingredient | Amount (mg/capsule) |
|---|---|
| Structure I (Racemic) | 100.0 |
| Starch | 407.0 |
| Magnesium stearate | 3.0 |

The active ingredient, cellulose, starch, and magnesium stearate are blended, passed through a No. 20 mesh U.S. sieve, and filled into hard gelatin capsules in 510 mg quantities.

### Extended-Release Formulations

Depending on the desired release profile, the oral solid dosage forms of the present invention may contain a suitable amount of controlled-release agents, extended-release agents, and/or modified-release agents (for example, delayed-release agents). The pharmaceutical solid oral dosage forms comprising the active agents of the present invention described herein can be further formulated to provide a modified or controlled release of the active agents of the present invention. In some embodiments, the solid dosage forms described herein can be formulated as a delayed release dosage form such as an enteric-coated delayed release oral dosage forms, i.e., as an oral dosage form of a pharmaceutical composition as described herein which uses an enteric coating to affect release in the small intestine of the gastrointestinal tract. The enteric-coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated. Enteric coatings may also be used to prepare other controlled release dosage forms including extended-release and pulsatile release dosage forms.

In other embodiments, the active agents of the formulations described herein are delivered using a pulsatile dosage form. Pulsatile dosage forms comprising the active agents of the present invention formulations described herein may be administered using a variety of formulations known in the art. For example, such formulations include those described in U.S. Pat. Nos. 5,011,692, 5,017,381, 5,229,135, and 5,840,329. Other dosage forms suitable for use with the active agents of the present invention formulations are described in, for example, U.S. Pat. Nos. 4,871,549, 5,260,068, 5,260,069, 5,508,040, 5,567,441 and 5,837,284.

In some embodiments, the controlled release dosage form is pulsatile release solid oral dosage form comprising at least two groups of particles, each containing active agents of the present invention as described herein. The first group of particles provides a substantially immediate dose of the active agents of the present invention upon ingestion by a subject. The first group of particles can be either uncoated or comprise a coating and/or sealant. The second group of particles comprises coated particles, which may comprise from about 2% to about 75%, typically from about 2.5% to about 70%, or from about 40% to about 70%, by weight of the total dose of the active agents of the present invention in the formulation, in admixture with one or more binders.

Coatings for providing a controlled, delayed, or extended-release may be applied to a compound, pure enantiomer or enantiomerically enriched mixture of Formula I, II, or III, or any one of Structures I-XLV or to a core containing a compound, pure enantiomer or enantiomerically enriched mixture of Formula I, II, or III, or any one of Structures I-XLV. The coating may comprise a pharmaceutically acceptable ingredient in an amount sufficient, for example, to provide an extended release from, for example, about 1 hours to about 7 hours following ingestion before release of a compound, pure enantiomer or enantiomerically enriched mixture of Formula I, II, or III, or any one of Structures-VI. Suitable coatings include one or more differentially degradable coatings such as, by way of example only, pH-sensitive coatings (enteric coatings) such as acrylic resins (for example, Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, and Eudragit® NE30D, Eudragit® NE 40D®) either alone or blended with cellulose derivatives, for example, ethylcellulose, or non-enteric coatings having variable thickness to provide differential release of the active agents of the present invention formulation.

Many other types of controlled/delayed/extended-release systems known to those of ordinary skill in the art and are suitable for use with the active agents of the present invention formulations described herein. Examples of such delivery systems include polymer-based systems, such as polylactic and polyglycolic acid, polyanhydrides and polycaprolactone, cellulose derivatives (for example, ethylcellulose), porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, for example, Liberman et al., Pharmaceutical Dosage Forms, 2 Ed., Vol. 1, pp. 209-214 (1990); Singh et al., Encyclopedia of Pharmaceutical Technology, 2nd Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983.

### Systemic Formulations

The formulations of the present invention suitable for intramuscular, subcutaneous, or intravenous injection may comprise physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propylene glycol, polyethylene-glycol, glycerol, cremophor and the like), suitable mixtures thereof, vegetable oils (such as olive oil) and injectable organic esters such as ethyl oleate. Additionally, the active agents of the present invention can be dissolved at concentrations of >1 mg/ml using water-soluble beta cyclodextrins (for example, beta-sulfobutyl-cyclodextrin and 2-hydroxypropyl-betacyclodextrin. Proper fluidity can be maintained, for example, by the use of a coating such as a lecithin, by the maintenance of the required particle size in the case of dispersions, and by the use of surfactants.

The formulations of the present invention suitable for subcutaneous injection may also contain additives such as preserving, wetting, emulsifying, and dispensing agents. Prevention of the growth of microorganisms can be ensured by various antibacterial and antifungal agents, such as parabens, benzoic acid, benzyl alcohol, chlorobutanol, phenol, sorbic acid, and the like. It may also be desirable to include isotonic agents, such as sugars, sodium chloride, and the like. Prolonged drug absorption of the injectable pharmaceutical form can be brought about by the use of agents delaying absorption, such as aluminum monostearate and gelatin. The active agents of the present invention in suspension formulations designed for extended-release via subcutaneous or intramuscular injection can avoid first-pass metabolism and lower dosages of the active agents of the present invention will be necessary to maintain plasma levels of about 50 ng/ml. In such formulations, the particle size of the active agents of the present invention particles and the range of the particle sizes of the active agents of the present invention particles can be used to control the release of the drug by controlling the rate of dissolution in fat or muscle.

In certain embodiments of the present invention, pharmaceutical compositions containing a compound, pure enantiomer or enantiomerically enriched mixture of Formula I, II, or III, or any one of Structures I-XLV may be formulated into a dosage form suitable for parenteral use. For example, the dosage form may be a lyophilized powder, a solution, suspension (for example, depot suspension).

In other embodiments, pharmaceutical compositions containing a compound, pure enantiomer or enantiomerically enriched mixture of Formula I, II, or III, or any one of Structures I-XLV may be formulated into a topical dosage form such as, but not limited to, a patch, a gel, a paste, a cream, an emulsion, liniment, balm, lotion, and ointment.

Another typical formulation employed in the methods of the present invention employs transdermal delivery devices (“patches”). Such transdermal patches may be used to provide continuous or discontinuous infusion of a compound of the present invention in controlled amounts. The construction and use of transdermal patches for the delivery of pharmaceutical agents is well known in the art. Such patches may be constructed for continuous, pulsatile, or on demand delivery of pharmaceutical agents.

Frequently, it will be desirable or necessary to introduce the pharmaceutical composition to the brain, either directly or indirectly. Direct techniques usually involve placement of a drug delivery catheter into the host's ventricular system to bypass the blood-brain barrier. Indirect techniques usually involve formulating the compositions to provide for drug latentiation by the conversion of hydrophilic drugs into lipid-soluble drugs or prodrugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions which can transiently open the blood-brain barrier.

### Non-Limiting Examples of Formulations for Systemic Delivery

The examples below provide non-limiting embodiments of formulations, which can be used to deliver any of a compound described herein in enantiomerically enriched form, pure form or even a racemic mixture. Therefore, while the compounds below are specified, any desired purity form or compound can be used if it achieves the desired goal of treatment.

A dry powder inhaler formulation is prepared containing the following components:

| Ingredient | Weight % |
|---|---|
| Structure I (R-enantiomer or S- | 5 |
| enantiomer) | |
| Lactose | 95 |

The active mixture is mixed with the lactose and the mixture is added to a dry powder inhaling appliance.

Suppositories, each containing 25 mg of active ingredient are made as follows:

| Ingredient | Quantity (mg) |
|---|---|
| Structure I (R-enantiomer or S-enantiomer) | 25.0 |
| Saturated fatty acid glycerides | 2000.0 |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

Suspensions, each containing 50 mg of active ingredient per 5.0 ml dose are made as follows:

| Ingredient | Amount | |
|---|---|---|
| Structure I (R-enantiomer or S-enantiomer) | 50.0 | mg |
| Xanthan gum | 4.0 | mg |
| Sodium carboxymethyl cellulose (11%) | 50.0 | mg |
| Microcrystalline cellulose (89%) | 50 | mg |
| Sucrose | 1.75 | g |
| Sodium benzoate | 10.0 | mg |
| Flavor and Color | q.v. | |
| Purified water | To 5.0 | ml |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

An intravenous formulation may be prepared as follows:

| Ingredient | Amount | |
|---|---|---|
| Structure II (Enantiomerically enriched S- | 250.0 | mg |
| enantiomer) | | |
| Isotonic saline | 1000 | ml |

A topical formulation may be prepared as follows:

| Ingredient | Amount (g) |
|---|---|
| Structure II (Enantiomerically enriched R- | 10.0 |
| enantiomer) | |
| Emulsifying Wax | 30.0 |
| Liquid Paraffin | 20.0 |
| White Soft Paraffin | To 100 |

The white soft paraffin is heated until molten. The liquid paraffin and emulsifying wax are incorporated and stirred until dissolved. The active ingredient is added and stirring is continued until dispersed. The mixture is then cooled until solid.

Sublingual or buccal tablets, each containing 20 mg of active ingredient, may be prepared as follows:

| Ingredient | Amount (mg/tablet) |
|---|---|
| Structure I (Enantiomerically enriched R- | 20.0 |
| enantiomer) | |
| Glycerol | 210.5 |
| Water | 143.0 |
| Sodium Citrate | 4.5 |
| Polyvinyl Alcohol | 26.5 |
| Polyvinylpyrrolidone | 15.5 |

The glycerol, water, sodium citrate, polyvinyl alcohol, and polyvinylpyrrolidone are admixed together by continuous stirring and maintaining the temperature at about 90° C. When the polymers have gone into solution, the solution is cooled to about 50-55° C. and the medicament is slowly admixed. The homogenous mixture is poured into forms made of an inert material to produce a drug-containing diffusion matrix having a thickness of about 2-4 mm. This diffusion matrix is then cut to form individual tablets having the appropriate size.

A liquid formulation is prepared containing the following components:

| Ingredient | Quantity (units) | |
|---|---|---|
| Structure I (Enantiomerically enriched-S- | 500 | mg |
| enantiomer) | | |
| Propylene Glycol | 2 | ml |
| Glycerin | 2 | ml |

The active mixture is mixed and added to a liquid vaporization appliance.

### Pharmaceutically Acceptable Salts

A compound of the present invention is an amine and thus basic, and therefore, reacts with inorganic and organic acids to form pharmaceutically acceptable acid addition salts. In some embodiments, a compound of the present invention as free amines is oily and has decreased stability at room temperature. In this case it may be beneficial to convert the free amine to a pharmaceutically acceptable acid addition salt for ease of handling and administration because in some embodiments, the pharmaceutically acceptable salt is solid at room temperature.

A compound described herein, including an enantiomerically enriched mixture, can be administered if desired as a pharmaceutically acceptable salt or a salt mixture. A salt mixture may be useful to increase solubility of the active substances, to alter pharmacokinetics, or for controlled release or other objective. A salt mixture may comprise 2, 3, 4, 5, 6, or more pharmaceutically acceptable salts together to form a single composition.

Acids commonly employed to form such salts are inorganic acids such as hydrochloric acid, hydrobromic acid, hydroiodic acid, sulfuric acid, phosphoric acid, and the like, and organic acids, such as p-toluenesulfonic acid, methanesulfonic acid, oxalic acid, p-bromophenylsulfonic acid, carbonic acid, succinic acid, citric acid, benzoic acid, acetic acid and the like.

Exemplary salts include 2-hydroxyethanesulfonate, 2-naphthalenesulfonate, 2-napsylate, 3-hydroxy-2-naphthoate, 3-phenylpropionate, 4-acetamidobenzoate, acefyllinate, acetate, aceturate, adipate, alginate, aminosalicylate, ammonium, amsonate, ascorbate, aspartate, benzenesulfonate, benzoate, besylate, bicarbonate, bisulfate, bitartrate, borate, butyrate, calcium edetate, calcium, camphocarbonate, camphorate, camphorsulfonate, camsylate, carbonate, cholate, citrate, clavulariate, cyclopentanepropionate, cypionate, d-aspartate, d-camsylate, d-lactate, decanoate, dichloroacetate, digluconate, dodecylsulfate, edentate, edetate, edisylate, estolate, esylate, ethanesulfonate, ethyl sulfate, finnarate, fumarate, furate, fusidate, galactarate (mucate), galacturonate, gallate, gentisate, gluceptate, glucoheptanoate, gluconate, glucuronate, glutamate, glutarate, glycerophosphate, glycolate, glycollylarsanilate, hemisulfate, heptanoate (enanthate), heptanoate, hexafluorophosphate, hexanoate, hexylresorcinate, hippurate, hybenzate, hydrabamine, hydrobromide, hydrobromide/bromide, hydrochloride, hydroiodide, hydroxide, hydroxybenzoate, hydroxynaphthoate, iodide, isethionate, isothionate, 1-aspartate, 1-camsylate, 1-lactate, lactate, lactobionate, laurate, laurylsulphonate, lithium, magnesium, malate, maleate, malonate, mandelate, meso-tartrate, mesylate, methanesulfonate, methylbromide, methylnitrate, methylsulfate, mucate, myristate, N-methylglucamine ammonium salt, napadisilate, naphthylate, napsylate, nicotinate, nitrate, octanoate, oleate, orotate, oxalate, p-toluenesulfonate, palmitate, pamoate, pantothenate, pectinate, persulfate, phenylpropionate, phosphate, phosphateldiphosphate, picrate, pivalate, polygalacturonate, potassium, propionate, pyrophosphate, saccharate, salicylate, salicylsulfate, sodium, stearate, subacetate, succinate, sulfate, sulfosaliculate, sulfosalicylate, suramate, tannate, tartrate, teoclate, terephthalate, thiocyanate, thiosalicylate, tosylate, tribrophenate, triethiodide, undecanoate, undecylenate, valerate, valproate, xinafoate, zinc and the like. (See Berge et al. (1977) “Pharmaceutical Salts,” J. Pharm. Sci. 66:1-19.) Most typical pharmaceutically acceptable salts are those employing a hydrochloride anion.

### Prodrugs

One of ordinary skill would understand that a compound, pure enantiomer or enantiomerically enriched mixture of the invention shall also include the prodrugs thereof. Prodrugs are compounds that are metabolized or otherwise transformed inside the body to the active pharmacologic agent(s) of interest. Thus, prodrug will contain the “active” component (for example, a compound, pure enantiomer or enantiomerically enriched mixture of any one of Structures I-XLV or Formula I, II, or III. Examples include N-alpha-acyloxyalkoxycarbonyl derivatives or addition of amino acids to the amine, which can be removed within the body by esterases or similar enzymes, but other prodrugs and precursors should be understood to be within the scope of the invention.

### Combination Therapy

It should be apparent that the compositions of the invention are not limited to combinations of a single active compound (i.e., one of Structures I or II), and a single carrier, diluent, or excipient alone, but also include combinations of multiple such Structures, other active compounds, and/or multiple carriers, diluents, and excipients. Pharmaceutical compositions of this invention thus may comprise one or more Structures (or their derivatives and analogues) in combination, together with one or more pharmaceutically-acceptable carriers, diluents, and/or excipients, and additionally with one or more other active compounds.

Different embodiments of the invention include the following examples: Pharmaceutically acceptable complex derivatives of each drug in each group, including solvates, salts, esters, enantiomers, isomers (stereoisomers and/or constitutional, including ones based on substituting deuterium for hydrogen), derivatives or prodrugs of a compound, pure enantiomer or enantiomerically enriched mixture of Formula I, II, or III, or any one of Structures I-XLV. Other embodiments of the invention include multiple variations in the pharmaceutical dosages of each drug in the combination as further outlined below. Other embodiments of the invention include various forms of preparations including using solids, liquids, immediate or delayed or extended-release forms. Many types of variations are possible as known to those skilled in the art.

In some aspects, a compound of the present invention is formulated in a pharmaceutical preparation with other active compounds to increase therapeutic efficacy, decrease unwanted effects, increase stability/shelf-life, and/or alter pharmacokinetics. Such other active compounds include, but are not limited to antioxidants (such alpha-lipoate in acid or salt form, ascorbate in acid or salt form, selenium, or N-acetylcysteine); substrates or inhibitors of cytochrome p450 2D6 (such as dextromethorphan, fluoxetine, paroxetine, bupropion, duloxetine, or quinidine), H2-receptor agonists or antagonists (such as famotidine); stimulants (such as dextroamphetamine, amphetamine, lisdexamphetamine, methylphenidate, or methamphetamine); entactogens (such as MDMA, 3,4-methylenedioxy-N-ethylamphetamine, [1-(2H-1,3-benzodioxol-5-yl)butan-2-yl](methyl)amine, 1-(1-benzofuran-6-yl)propan-2-amine, or [1-(1-benzofuran-5-yl)propan-2-yl](methyl)amine); anti-inflammatories (such as ibuprofen or ketoprofen); matrix metalloproteinase inhibitors (such as doxycycline); NOS inhibitors (such as S-methyl-L-thiocitrulline); proton pump inhibitors (such as omeprazole); phosphodiesterase 5 inhibitors (such as sildenafil); drugs with cardiovascular effects (beta antagonists such as propranolol, mixed alpha and beta antagonists such as carvedilol, alpha antagonists such as prazosin, imidazoline receptor agonists such as rilmenidine or moxonidine; serotonin antagonists such as ketanserin or lisuride); norepinephrine transporter blockers (such as reboxetine); acetylcholine nicotinic receptor modulators (such as bupropion, hydroxybupropion, methyllycaconitine, memantine, or mecamylamine); gastrointestinal acidifying agents (such as ascorbic acid or glutamic acid hydrochloride); alkalinizing agents (such as sodium bicarbonate), NMDA receptor antagonists (such as ketamine); TrkB agonists (such as 7,8-dihydroxyflavone, 7,8,3′-trihydroxyflavone, or N-acetylserotonin), or serotonin receptor agonists (such as 5-methoxy-N-methyl-N-isopropyltryptamine, N,N-Dimethyl-2-(2-methyl-1H-indol-1-yl)ethan-1-amine, psilocin, or psilocybin). The ingredients may be in ion, freebase, or salt form and may be isomers or prodrugs.

The pharmacological agents that make up the combination therapy disclosed herein may be a combined dosage form or in separate dosage forms intended for substantially simultaneous administration. The pharmacological agents that make up the combination therapy may also be administered sequentially, with either therapeutic compound being administered by a regimen calling for two-step administration. The two-step administration regimen may call for sequential administration of the active agents or spaced-apart administration of the separate active agents.

The time period between the multiple administration steps may range from, a few minutes to several hours, depending upon the properties of each pharmacological agent, such as potency, solubility, bioavailability, plasma half-life and kinetic profile of the pharmacological agent. Circadian variation of the target molecule concentration may also determine the optimal dose interval. For example, a compound of the present invention may be administered while the other pharmacological agent is being administered (concurrent administration) or may be administered before or after other pharmacological agent is administered (sequential administration).

In cases where the two (or more) drugs included in the fixed-dose combinations of the present invention are incompatible, cross-contamination can be avoided, for example, by incorporation of the drugs in different drug layers in the oral dosage form with the inclusion of a barrier layer(s) between the different drug layers, wherein the barrier layer(s) comprise one or more inert/non-functional materials.

In certain typical embodiments, the formulations of the present invention are fixed-dose combinations of any one of Structures I-XLV and at least one other pharmacological agent. In certain typical embodiments, the formulations of the present invention are fixed-dose combinations of a compound, pure enantiomer or enantiomerically enriched mixture of Formula I II, or III and at least one other pharmacological agent. Fixed-dose combination formulations may contain therapeutically efficacious fixed-dose combinations of formulations of a compound, pure enantiomer or enantiomerically enriched mixture of Formula I, II, or III, or any one of Structures I-XLV and other pharmacological agents in the form of single-layer monolithic tablet or multi-layered monolithic tablet or in the form of a core tablet-in-tablet or multi-layered multi-disk tablet or beads inside a capsule or tablets inside a capsule.

Pharmaceutical Combinations with Dextroamphetamine

In certain typical embodiments, a compound, pure enantiomer or enantiomerically enriched mixture of Formula I, II, or III, or any one of Structures I-XLV, either racemic, an enantiomer, or a mixture of enantiomers, and with zero or more hydrogens replaced with deuterium, is formulated in a pharmaceutical composition that contains a pharmaceutically acceptable salt of dextroamphetamine, for example, in the amount between about 2 mg to 25 mg, such as, 2 mg, 4 mg, 5 mg, 7 mg, 10 mg, 15 mg, 20 mg, or 25 mg. The required amount of dextroamphetamine will vary depending on the needs of the patient.

In another typical embodiment, a compound, pure enantiomer or enantiomerically enriched mixture of Formula I, II, or III, or any one of Structures I-XLV, either racemic, an enantiomer, or a mixture of enantiomers, and with zero or more hydrogens replaced with deuterium, are formulated in a pharmaceutical composition that contains a pharmaceutically acceptable salt of dextroamphetamine with dextroamphetamine, for example, in a ratio by weight of 1:2, 1:3, 1:4, 1:5, 1:6, 1:7, 1:8, 1:9 or 1:10 to the compound, pure enantiomer or enantiomerically enriched mixture of Formula I, II, or III, or any one of Structures I-XLV. The required amount of dextroamphetamine will vary depending on the needs of the patient.

Pharmaceutical Combinations with MDMA

In some typical embodiments, a compound, pure enantiomer or enantiomerically enriched mixture of Formula I, II, or III, or any one of Structures I-XLV, either racemic, an enantiomer, or a mixture of enantiomers, and with zero or more hydrogens replaced with deuterium, is formulated in a pharmaceutical composition that contains a pharmaceutically acceptable salt of MDMA, for example, in an amount between 5 and 180 mg, typically 15-60 mg. MDMA may be racemic, a pure enantiomer (such as the S-enantiomer), or enantiomerically enriched. The required amount of MDMA will vary depending on the needs of the patient.

In some typical embodiments, a compound, pure enantiomer or enantiomerically enriched mixture of Formula I, II, or III, or any one of Structures I-XLV, either racemic, an enantiomer, or a mixture of enantiomers, and with zero or more hydrogens replaced with deuterium, is formulated in a pharmaceutical composition that contains a pharmaceutically acceptable salt of MDMA with MDMA, for example, in a ratio by weight of 1:2, 1:3, 1:4, 1:5, 1:6, 1:7, 1:8, 1:9 or 1:10 to the compound, pure enantiomer or enantiomerically enriched mixture of Formula I, II, or III, or any one of Structures I-XLV. The required amount of MDMA will vary depending on the needs of the patient.

### Non-Limiting Examples of Combination Formulations

Capsules, each containing 40 mg of the current invention, are made as follows:

| Ingredient | Quantity (mg/capsule) |
|---|---|
| Structure I (R- or S-enantiomer, D3-N- | 10.0 |
| Deuterated) | |
| Structure II (R- or S-enantiomer, D3-N- | 30.0 |
| Deuterated) | |
| Amphetamine sulfate | 5.0 |
| Starch | 109.0 |
| Magnesium stearate | 1.0 |

The active ingredients, cellulose, starch, and magnesium stearate are blended, passed through a No. 20 mesh U.S. sieve, and filled into hard gelatin capsules in 155 mg quantities.

Capsules, each containing 40 mg of the current invention, are made as follows:

| Ingredient | Quantity (mg/capsule) |
|---|---|
| Structure I (70% R-or S-enantiomer) | 30.0 |
| Structure II (70% S- or R-enantiomer) | 10.0 |
| Psilocybin hydrochloride | 2.0 |
| Alpha lipoic acid | 40.0 |
| Starch | 72.0 |
| Magnesium stearate | 1.0 |

The active ingredients, cellulose, starch, and magnesium stearate are blended, passed through a No. 20 mesh U.S. sieve, and filled into hard gelatin capsules in 155 mg quantities.

It should be readily appreciated that the formulation examples are illustrative only. Accordingly, it should be understood that reference to particular Structure(s) is likewise illustrative, and the Structure(s) in any of the non-limiting examples may be substituted by other Structure(s) of the invention. Likewise, any of the other active compounds (for example, amphetamine sulfate or psilocybin hydrochloride) may be substituted by a different other active compound, as may be the inactive compounds.

Moreover, for any active compound of the invention, for example Structure I or Structure II, substitution of the compound by its prodrug, free base, salt, or hydrochloride salt shall be understood to provide merely an alternative embodiment still within the scope of the invention. Further, compositions within the scope of the invention should be understood to be open-ended and may include additional active or inactive compounds and ingredients.

The type of formulation employed for the administration of a compound employed in the methods of the present invention generally may be dictated by the compound(s) employed, the type of pharmacokinetic profile desired from the route of administration and the compound(s), and the state of the patient.

### Dosage Regimes

A compound or pharmaceutically acceptable formulation of the present invention can be administered to the host in any amount, and with any frequency, that achieves the goals of the invention as used by the healthcare provider, or otherwise by the host in need thereof, typically a human, as necessary or desired.

In certain embodiments, the composition as described herein is provided only in a controlled counseling session, and administered only once, or perhaps 2, 3, 4, or 5 or more times in repeated counseling sessions to address a mental disorder as described herein.

In other embodiments, the composition as described herein is provided outside of a controlled counseling session, and perhaps self-administered, as needed to perhaps 2, 3, 4, or 5 or more times in to address a mental disorder as described herein.

In other embodiments, the composition of the present invention may be administered on a routine basis for mental wellbeing or for entactogenic treatment.

A compound of the current invention can be administered in a variety of doses, routes of administration, and dosing regimens, based on the indication and needs of the patient. Non-limiting examples of therapeutic use include discrete psychotherapeutic sessions, ad libitum use for treatment of episodic disorders, and ongoing use for treatment of subchronic and chronic disorders.

### Psychotherapeutic Sessions

For some indications, the selected indolizine medicine of the present invention is taken in discrete psychotherapy or other beneficial sessions. It is anticipated that these sessions will typically be separated by more than 5 half-lives of the medicine and, for most patients, will typically occur only 1 to 5 times each year.

For these sessions, it will typically be desirable to induce clearly perceptible entactogenic effects that will facilitate fast therapeutic progress. Non-exhaustive examples of oral doses of medicine that produce clearly perceptible entactogenic effects for exemplary purposes for any compound described herein includes (using compounds for illustrative purposes only): about 40 to about 120 mg of any one of non-racemic Structures I-XLV, about 40 to about 120 mg of any one of Structures I-XLV, about 50 to about 300 mg of any one of non-racemic Structures I-XLV, about 50 to about 300 mg any one of Structures I-XLV, about 75 to about 500 mg any one of non-racemic Structures I-XLV, about 75 to about 500 mg of any one of Structures I-XLV, about 75 to about 800 mg of any one of Structures I-XLV, about 75 to about 800 mg any one of non-racemic Structures I-XLV. Non-exhaustive examples of oral doses of medicine that produce clearly perceptible entactogenic effects for exemplary purposes for any compound described herein includes (using compounds for illustrative purposes only): about 40 to about 120 mg of non-racemic Formula I, II, or III, about 50 to about 300 mg of Formula I, II, or III, about 75 to about 500 mg of Formula I, II, or III, about 75 to about 800 mg of Formula I, II, or III.

Additional non-exhaustive examples of oral doses of medicine that produce clearly perceptible entactogenic effects for exemplary purposes for any compound described herein includes (using compounds for illustrative purposes only): at least about 40 or not more than about 120 mg of any one of non-racemic Structures I-XLV, at least about 40 or not more than about 120 mg of any one of Structures I-XLV, at least about 50 or not more than about 300 mg of any one of non-racemic Structures I-XLV, at least about 50 or not more than about 300 mg any one of Structures I-XLV, at least about 75 or not more than about 500 mg any one of non-racemic Structures I-XLV, at least about 75 or not more than about 500 mg of any one of Structures I-XLV, not more than about 800 mg of any one of Structures I-XLV, not more than about 800 mg any one of non-racemic Structures I-XLV. Non-exhaustive examples of oral doses of medicine that produce clearly perceptible entactogenic effects for exemplary purposes for any compound described herein includes (using compounds for illustrative purposes only): about 40 or about 120 mg of non-racemic Formula I, II, or III, about 50 or about 300 mg of Formula I, II, or III, about 75 or about 500 mg of Formula I, II, or III, about 75 or about 800 mg of Formula I, II, or III.

It is anticipated that the medicine would be taken once or, more rarely, two or three times in a single therapeutic session. In these cases, it is common for each subsequent dose to be half of the previous dose or lower. Multiple doses within a session typically occur because either the patient's sensitivity to the medicine was unknown and too low of an initial dose was employed or because the patient is experiencing a productive session and it is desirable to extend the duration of therapeutic effects. Controlled release preparations may be used to lengthen the duration of therapeutic effects from a single administration of the medicine. In cases where multiple administrations are used in a session, it is anticipated that individual doses will be lower so that plasma concentrations remain within a desired therapeutic range.

Non-limiting, non-exhaustive examples of indications that may benefit from psychotherapeutic sessions include post-traumatic stress disorder, depression, dysthymia, anxiety and phobia disorders, feeding, eating, and binge disorders, body dysmorphic syndromes, alcoholism, tobacco abuse, drug abuse or dependence disorders, disruptive behavior disorders, impulse control disorders, gaming disorders, gambling disorders, personality disorders, attachment disorders, autism, and dissociative disorders. Also included as exemplary situations where an individual would benefit from a psychotherapeutic session are situations from a reduction of neuroticism or psychological defensiveness, an increase in openness to experience, an increase in creativity, or an increase in decision-making ability.

### Ad Libitum Use for Treatment of Episodic Disorders

For some indications, such as social anxiety, where the patient has need for relief from episodic occurrence of a disorder, it is anticipated that the medicine would be taken as needed but that uses should be separated by more than 5 half-lives of the medicine to avoid bioaccumulation and formation of tolerance.

For treating episodic disorders, clearly perceptible entactogenic effects are often not desirable, as they can impair some aspects of functioning. Non-exhaustive examples of oral doses of medicine for any compound described herein includes (using compounds for illustrative purposes only) that produce subtle, barely perceptible therapeutic effects include: about 10 to about 60 mg of any one of non-racemic Structures I-XLV, about 10 to about 60 mg of any one of Structures I-XLV, about 10 to about 100 mg of any one of non-racemic Structures I-XLV, about 10 to about 100 mg any one of Structures I-XLV, about 20 to about 150 mg of any one of non-racemic Structures I-XLV, about 20 to about 150 mg of any one of Structures I-XLV, about 20 to about 200 mg of any one of non-racemic Structures I-XLV, and about 20 to about 200 mg of any one of Structures I-XLV. Non-exhaustive examples of oral doses of medicine for any compound described herein includes (using compounds for illustrative purposes only) that produce subtle, barely perceptible therapeutic effects include: about 10 to about 60 mg of non-racemic Formula I, II, or III, about 10 to about 100 mg of Formula I, II, or III, about 20 to about 150 mg of Formula I, II, or III, and about 20 to about 200 mg of Formula I, II, or III.

Non-limiting, non-exhaustive examples of indications that may benefit from episodic treatment include post-traumatic stress disorder, depression, dysthymia, anxiety and phobia disorders, feeding, eating, and binge disorders, body dysmorphic syndromes, alcoholism, tobacco abuse, drug abuse or dependence disorders, disruptive behavior disorders, impulse control disorders, gaming disorders, gambling disorders, personality disorders, attachment disorders, autism, and dissociative disorders, provided that clinically significant signs and symptoms worsen episodically or in predictable contexts.

### Ongoing Use for Treatment of Subchronic and Chronic Disorders

For some indications, such as substance use disorders, inflammatory conditions, chronic pain, and neurological indications, including treatment of stroke, brain trauma, dementia, and neurodegenerative diseases, where the patient has need for ongoing treatment, it is anticipated that the medicine would be taken daily, twice daily, or three times per day. With some indications (subchronic disorders), such as treatment of stroke or traumatic brain injury, it is anticipated that treatment duration will be time-limited and dosing will be tapered when the patient has recovered. An example dose taper regimen is a reduction in dose of 10% of the original dose per week for nine weeks. With other, chronic disorders, such as dementia, it is anticipated that treatment will be continued as long as the patient continues to receive clinically significant benefits.

For treating subchronic and chronic disorders, clearly perceptible entactogenic effects are often not desirable. Non-exhaustive examples of oral doses of medicine for any compound described herein includes (using compounds for illustrative purposes only) that produce subtle, barely perceptible therapeutic effects with ongoing dosing include: about 5 to about 60 mg of any one of non-racemic Structures I-XLV, about 5 to about 60 mg of any one of Structures I-XLV, about 5 to about 100 mg of any one of Structures I-XLV, about 5 to about 100 mg of any one of non-racemic Structures I-XLV, about 10 to about 150 mg of any one of Structures I-XLV, about 10 to about 150 mg of any one of non-racemic Structures I-XLV, about 10 to about 200 mg of any one of Structures I-XLV, and about 10 to about 200 mg of any one of non-racemic Structures I-XLV. Non-exhaustive examples of oral doses of medicine for any compound described herein includes (using compounds for illustrative purposes only) that produce subtle, barely perceptible therapeutic effects with ongoing dosing include: about 5 to about 60 mg of non-racemic Formula I, II, or III, about 5 to about 100 mg of Formula I, II, or III, about 10 to about 150 mg of Formula I, II, or III, and about 10 to about 200 mg of Formula I, II, or III.

Non-limiting, non-exhaustive examples of subchronic and chronic disorders that may benefit from regular treatment include migraine, headaches (for example, cluster headache), neurodegenerative disorders, Alzheimer's disease, Parkinson's disease, schizophrenia, stroke, traumatic brain injury, phantom limb syndrome, chronic pain syndromes, and other conditions where increasing neuronal plasticity is desirable.

### Synthetic Approaches for Compounds of the Present Invention

Methods for synthesis of the compounds described herein and/or starting materials are either described in the art or will be readily apparent to the skilled artisan in view of general references well-known in the art (see, e.g., Green et al., “Protective Groups in Organic Chemistry,” (Wiley, 2nd ed. 1991); Harrison et al., “Compendium of Synthetic Organic Methods,” Vols. 1-8 (John Wiley and Sons, 1971-1996); “Beilstein Handbook of Organic Chemistry,” Beilstein Institute of Organic Chemistry, Frankfurt, Germany; Feiser et al, “Reagents for Organic Synthesis,” Volumes 1-17, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” John Wiley & Sons, 1995) and may be used to synthesize the compounds of the invention. In general, the approaches used for similar compounds (Ghinea & Dinica. 2016. Scope of Selective Heterocycles from Organic and Pharmaceutical Perspective, 115-142; Ramalakshmi et al. 20201. Der PharmaChemica, 2021, 13(2): 60-69; Shulgin & Shulgin. 1992. PiHKAL. A chemical love story, Transform Press, Berkeley CA; Glennon et al. 1986. Journal of medicinal chemistry, 29(2), 194-199; Nichols et al. 1991. Journal of medicinal chemistry, 34(1), 276-281; Kedrowski et al. 2007. Organic Letters, 9(17), 3205-3207; Heravi & Zadsirjan. 2016. Current Organic Synthesis, 13(6), 780-833; Keri et al. 2017. European journal of medicinal chemistry, 138, 1002-1033; Perez-Silanes et al. 2001. Journal of Heterocyclic Chemistry, 38(5), 1025-1030; and references therein), such adaptation being that known and understood to those of ordinary skill.

### Example 1: Synthesis of Indolizines of the Present Invention

### Synthesis 1: Synthesis of 2-(indolizin-3-yl)ethan-1-amine—optionally substituted alpha to the amine

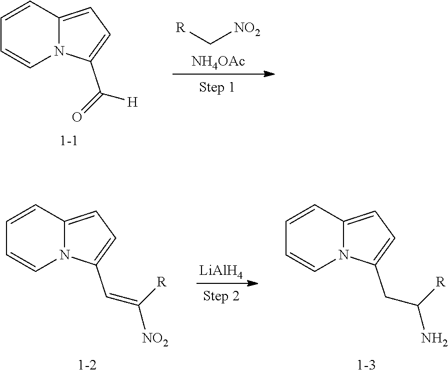

### Synthesis 2: Synthesis of 2-amino-1-(indolizin-3-yl)ethan-1-one—optionally substituted alpha to the amine

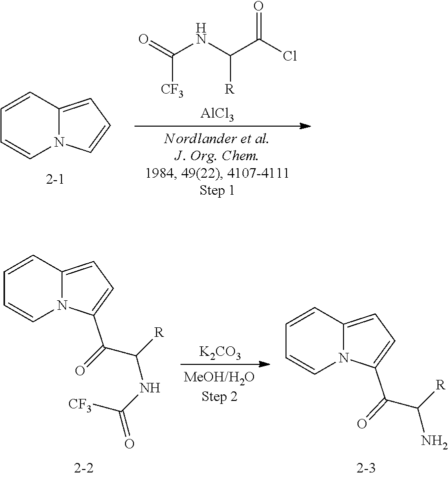

### Synthesis 3: Synthesis of 2-amino-1-(indolizin-3-yl)ethan-1-ol—optionally substituted alpha to the amine

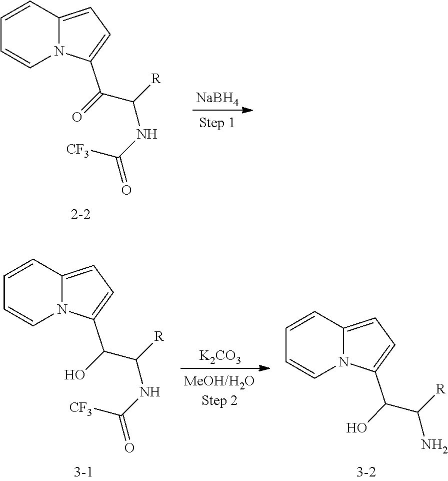

### Synthesis 4: Synthesis of 2-(indolizin-2-yl)ethan-1-amine—optionally substituted alpha to the amine

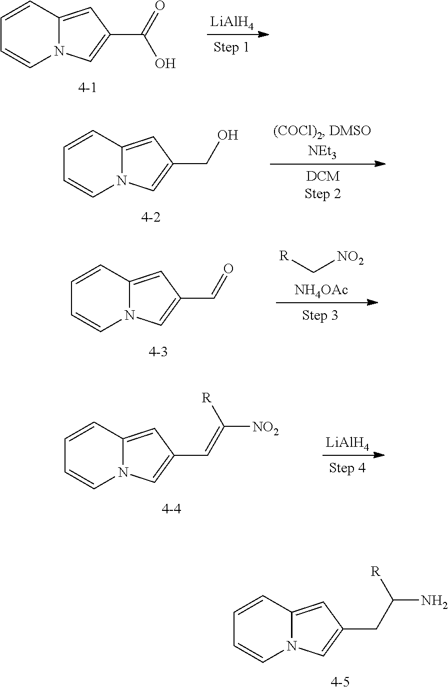

### Synthesis 5: Synthesis of 2-amino-1-(indolizin-2-yl)ethan-1-ol

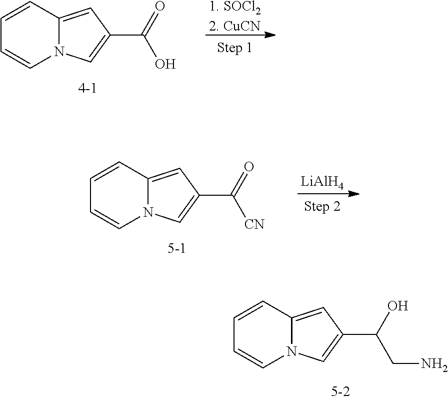 [SMILES: O=C(O)c1cc2ccccn2c1 ; "1. SOCl22. CuCN" ; N#CC(=O)c1cc2ccccn2c1 ; "LiAlH4" ; NCC(O)c1cc2ccccn2c1]

### Synthesis 6: Synthesis of 2-amino-1-(indolizin-2-yl)ethan-1-one

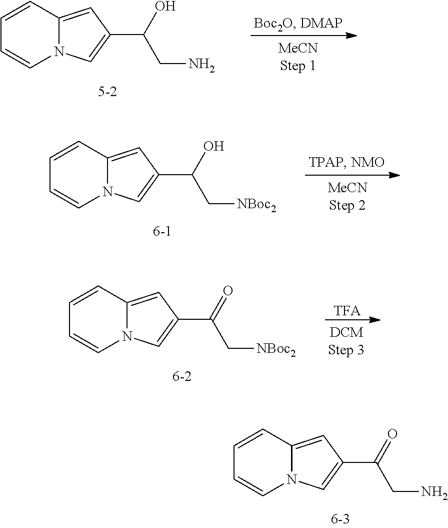

### Synthesis 7: Synthesis of 1-(indolizin-8-yl)-2-(methylamino)ethan-1-one—optionally substituted alpha to the amine

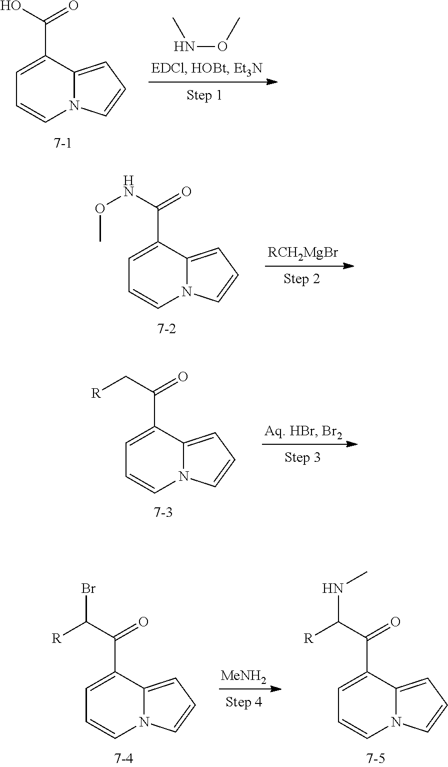

### Synthesis 8: Synthesis of 1-(indolizin-7-yl)-2-(methylamino)ethan-1-one—optionally substituted alpha to the amine

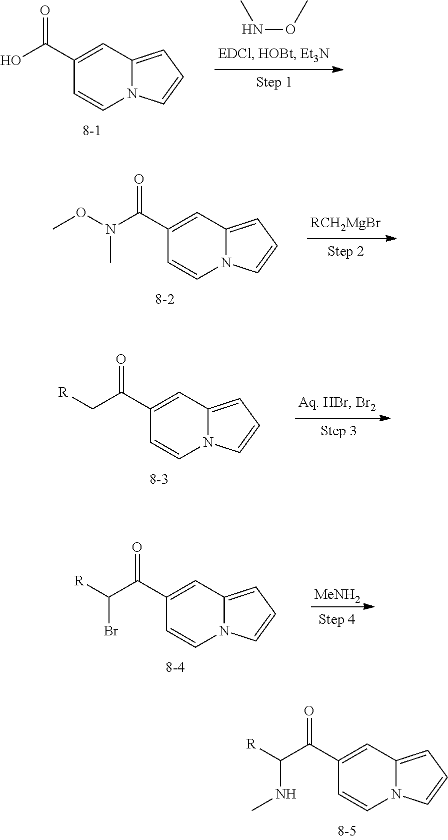

### Synthesis 9: Synthesis of 1-(indolizin-6-yl)-2-(methylamino)ethan-1-one—optionally substituted alpha to the amine

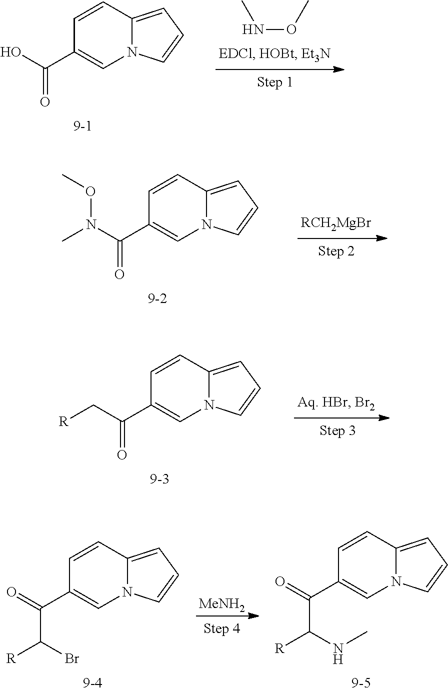

### Synthesis 10: Synthesis of 1-(indolizin-5-yl)-2-(methylamino)ethan-1-one—optionally substituted alpha to the amine

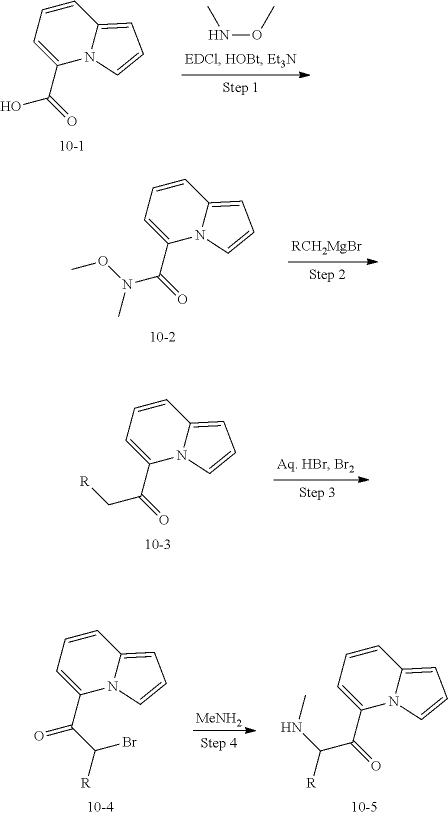

While some reaction sequences to form aminoalkyl groups of particular substitutions are only shown for a single heteroaryl regioisomer, the same reaction sequences can be similarly applied to any heteroaryl regioisomer by starting with the appropriate starting material. For example, other heteroaryl isomers with similar aminoalkyl groups can be made by starting with the appropriate regioisomer of the heteroaryl aldehyde or carboxylic acid. For example, the starting materials in Synthesis 11 are known and can be used to prepare compounds with various regiochemistry. Similarly, where R is used to denote a site of variation, a desired substitution can be made by selection of the appropriately substituted starting material.

### Synthesis 11: Synthesis of Various Beta-Ketones

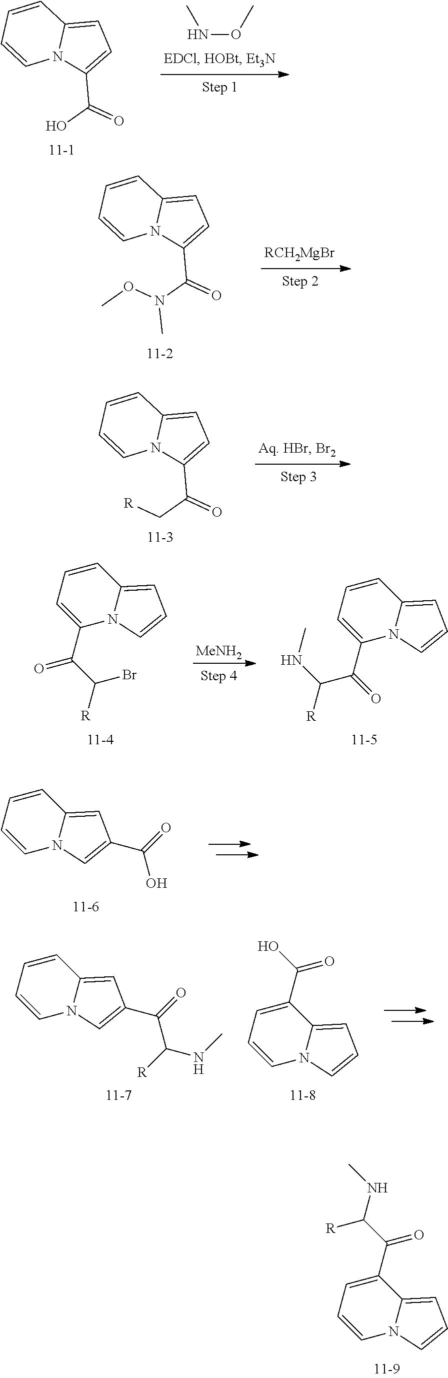

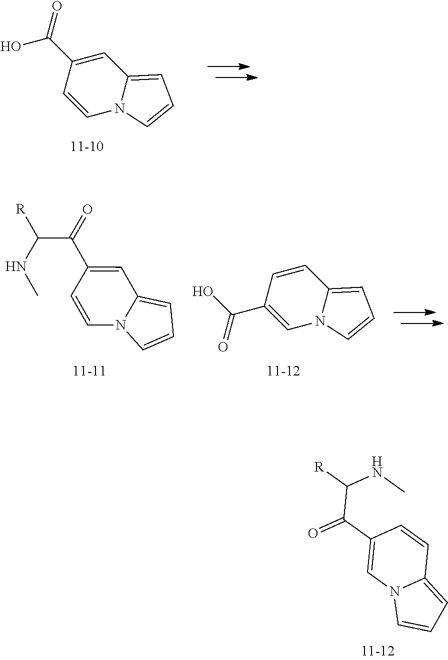

Different R groups can be incorporated into the molecule by selection of the Grignard reagent. For example, when a methyl group is desired at the alpha position a methyl Grignard reagent can be used and when an ethyl group is desired an ethyl Grignard reagent can be used.

### Synthesis 12: Synthesis of Alpha-Methyl Compounds from Weinreb Amide Intermediates

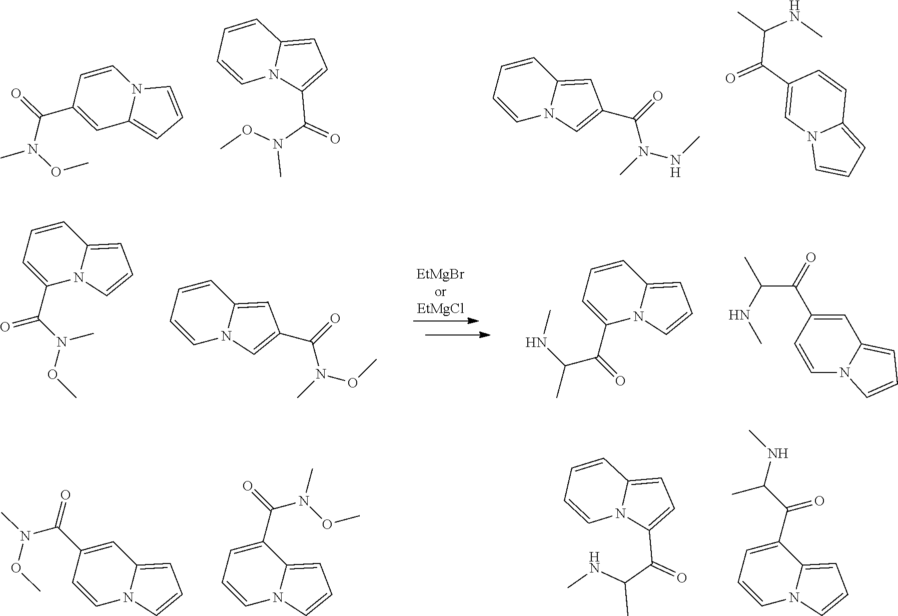

### Synthesis 13: Synthesis of Alpha-Ethyl Compounds from Weinreb Amide Intermediates

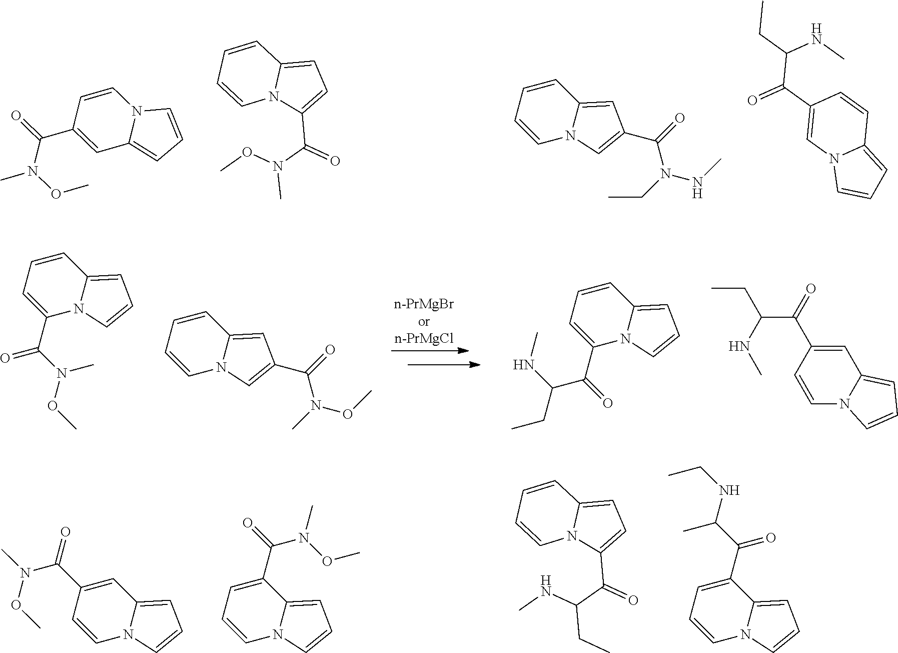

The primary and methyl amines drawn in the schemes above can be replaced with other amines such as ethylamine or dimethyl amine by replacing the starting amine material and modifying the reaction conditions if necessary. For example, when dimethyl amine is used instead of methyl amine a higher reaction temperature or stronger base may be utilized to increase reactivity.

### Synthesis 14: Synthesis of Various Different Amines

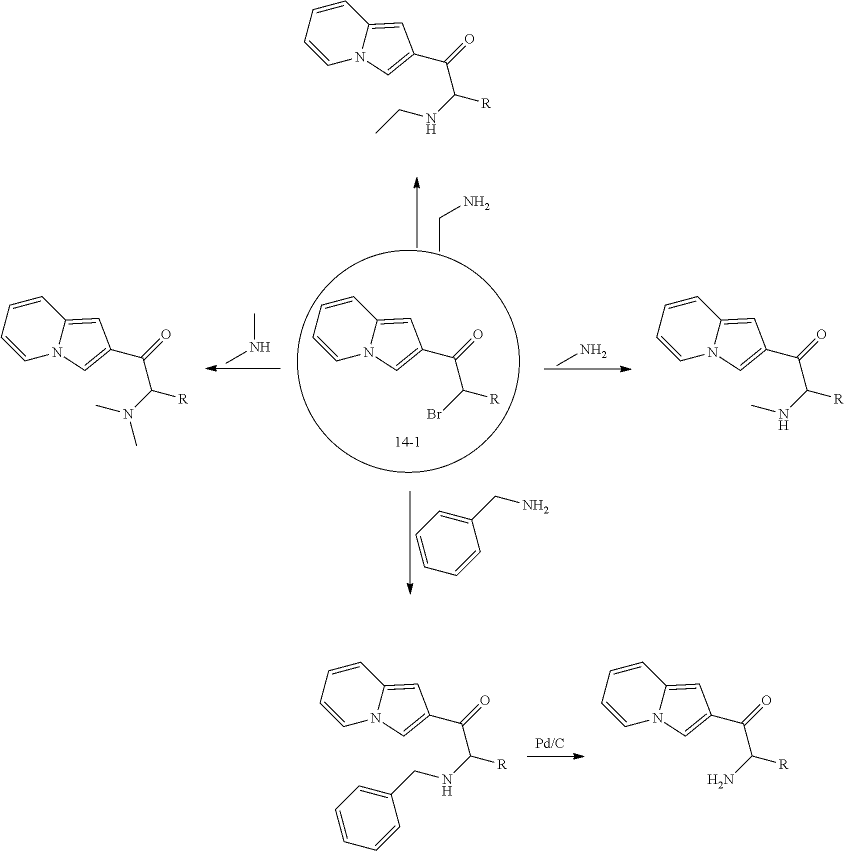

The chiral compounds of the present invention can be separated using the various chiral separation techniques discussed herein and otherwise known such as chiral HPLC. In other embodiments a compound is provided that is a diastereomer and the diastereomers can be separated using conventional techniques and then enantiomers can be further purified if desired.

### Synthesis 15: Chiral Separation 1

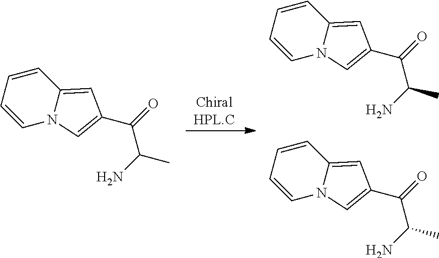

### Synthesis 16: Chiral Separation 2

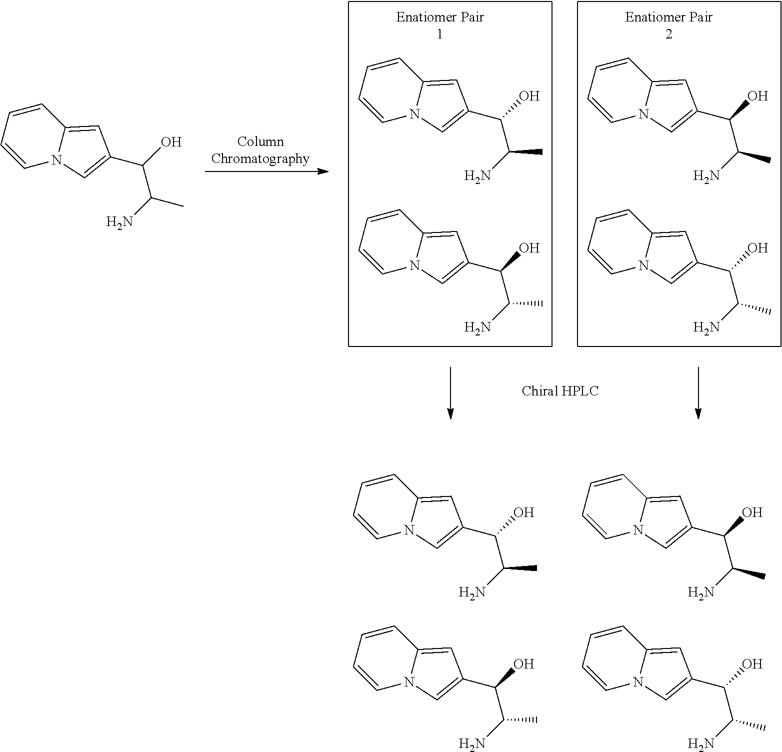

In other embodiments the chirality of the molecule can be set or enhanced by choice of synthetic conditions. For example, chiral reducing agents are well known and using bulky or small reducing agents can affect stereochemical selectivity (see synthesis 17). Alternatively other chiral reagents can be employed (see synthesis 18).

### Synthesis 17: Chiral Synthesis 1

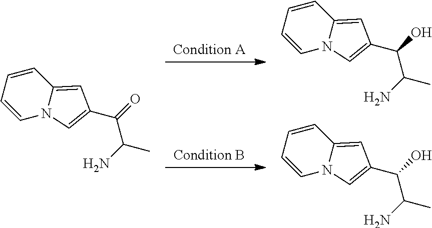

### Synthesis 18: Chiral Synthesis 2

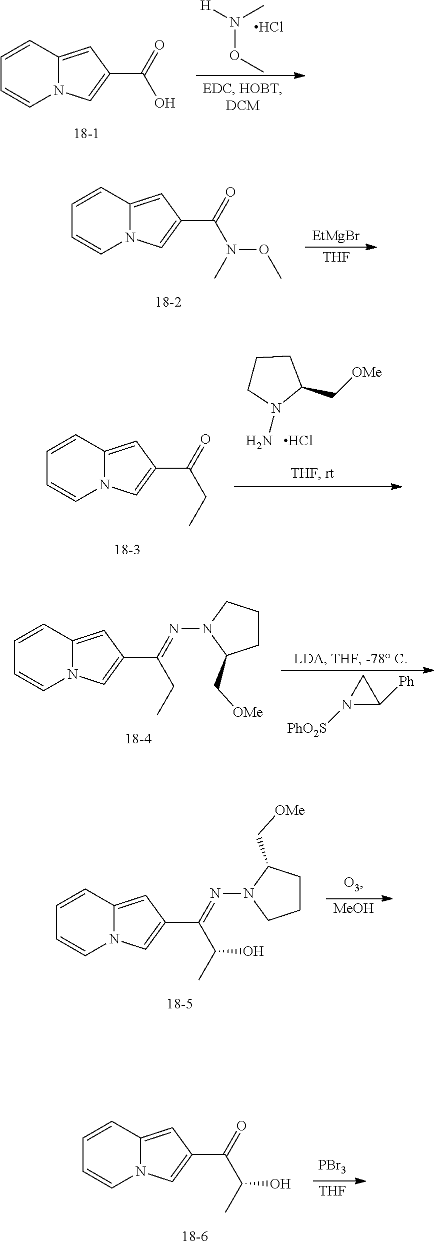

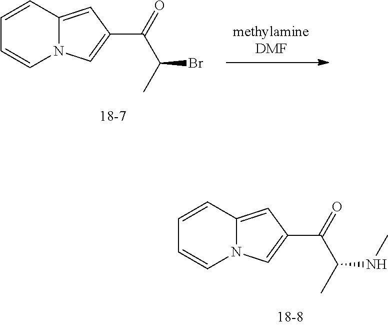

### Synthesis 19: Chiral Synthesis 3

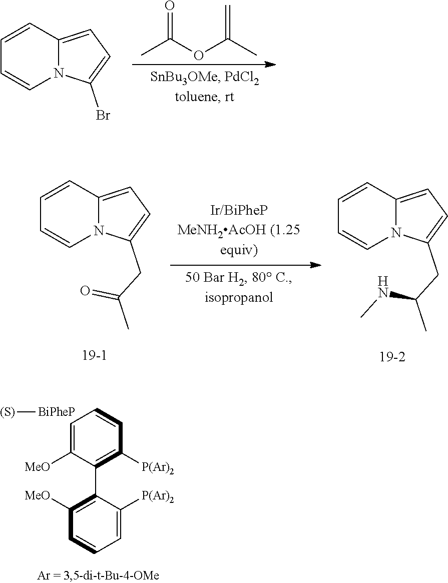

### Synthesis 20: Chiral Synthesis 5

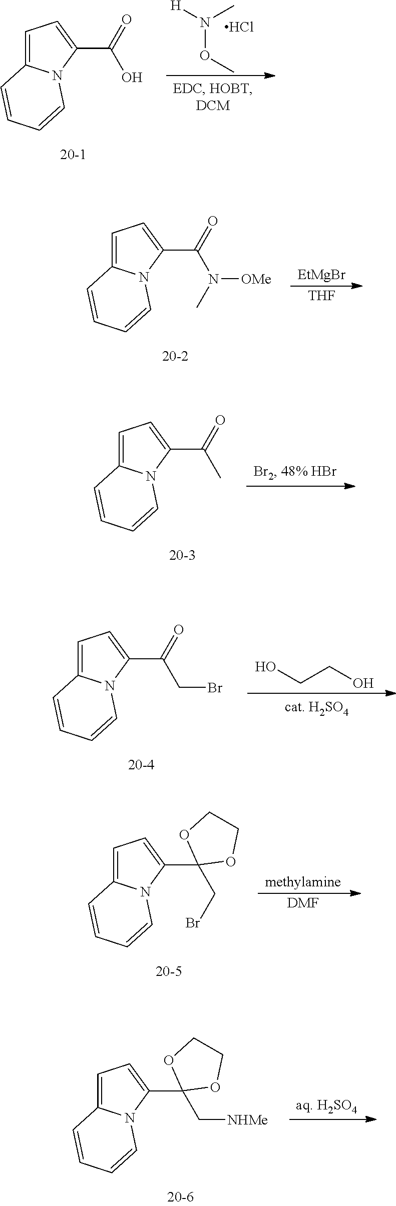

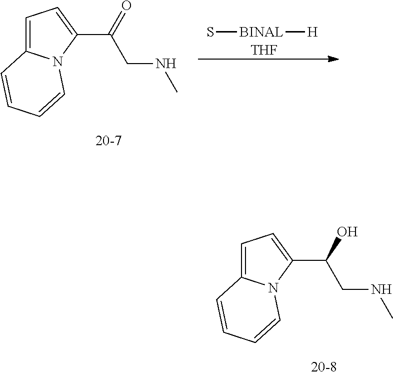

### Synthesis 21: Chiral Synthesis 5

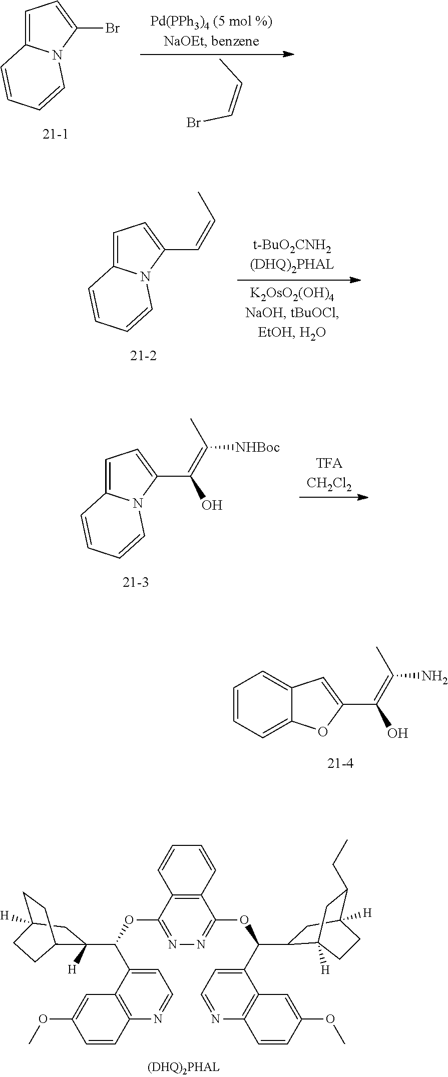

### Example 2: Synthesis of Indolizine Carbaldehyde Intermediates

### Synthesis 22: Synthesis of indolizine-1-carbaldehyde (22-6)

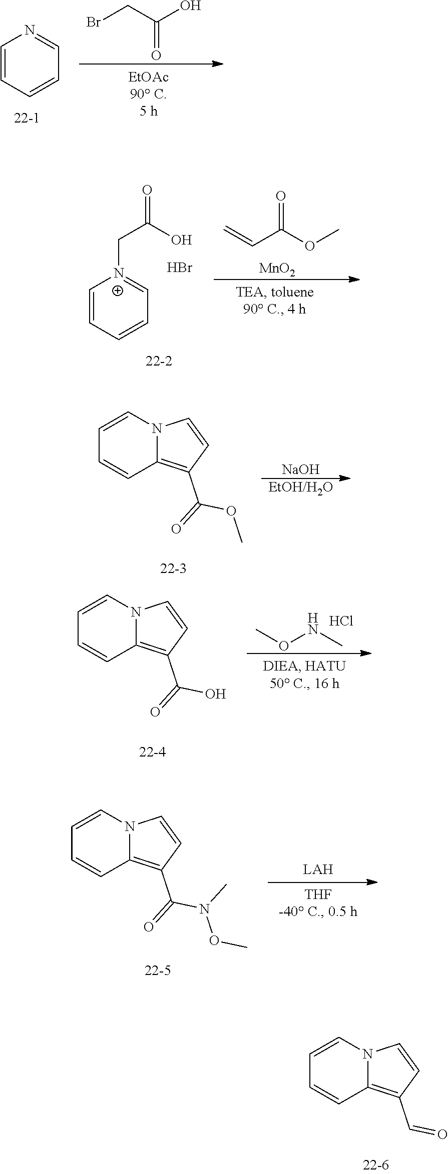

Pyridine (22-1, 15 g) was transformed to hydrobromide salt of 1-(carboxymethyl)pyridin-1-ium via nucleophilic substitution with 2-bromoacetic acid by heating the reaction mixture in ethyl acetate at 90° C. for 5 hours. Product Intermediate 22-2 was recovered with the yield of 16 g after work up. Intermediate 22-2 was converted to 22-3 by reacting it with methyl acrylate in the presence of triethylamine, manganese dioxide, and toluene as solvent at 90° C. for 4 hours. Crude was purified with column chromatography to yield 3.7 g of 22-3. Methyl ester indolizine 22-3 was hydrolyzed with sodium hydroxide in ethanol/water solvent resulting in 2.7 g of the indolizine carboxylic acid 22-4. Indolizine carboxylic acid 22-4 was transformed to N-methoxy-N-methylindolizine-1-carboxamide (22-5) in presence of N,O-dimethylhydroxylamine hydrochloride, diisopropylethylamine (DIEA), hexaluorophosphate azabenzotraizole tetramethyl uranium (HATU) at 50° C. After purification, 27% of the desired product (22-5) was obtained, which was hydrogenated with lithium aluminum hydride (LAH) in THF at −40° C. for 0.5 hours to yield indolizine-1-carbaldehyde (22-6).

### Synthesis 23: Synthesis of indolizine-2-carbaldehyde (23-5)

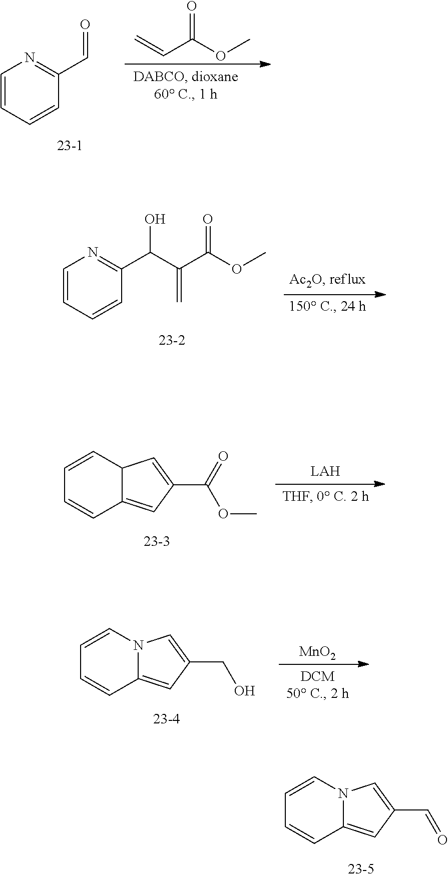

Picolinaldehyde (1 g) was transformed into methyl 2-(hydroxy(pyridin-2-yl)methyl)acrylate by reacting it with methyl acrylate in presence of 1,4-diazabicyclo[2.2.2]octane (DABCO) in dioxane solvent at 60° C. for 1 hour. Crude was purified with column chromatography resulting in 1.2 g of 23-2. Intermediate 23-2 was transformed into methyl ester indolizine, 23-3 by reacting it with acetic anhydride at 150° C. for 24 hours. Intermediate 23-3 (262 mg) was recovered after purification with column chromatography. Intermediate 23-3 was then hydrogenated to 23-4 with LAH in THF at 0° C. for 2 hours to afford 148 mg of material. Intermediate 23-4 can be oxidized to 23-5 by reacting with manganese dioxide in dichloromethane (DCM) at 50° C. for 2 hours.

### Synthesis 24: Synthesis of indolizine-3-carbaldehyde (24-4)

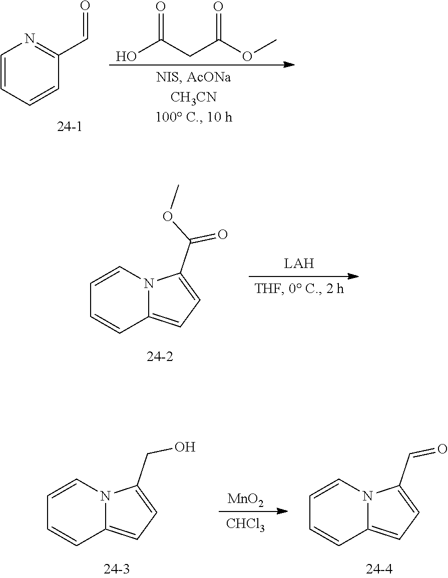

Picolinaldehyde (1 g) was transformed into methyl ester indolizine 24-2 by reacting it with 3-methoxy-3-oxopropanoic acid in presence of N-iodosuccinimide, sodium acetate in acetonitrile solvent at 100° C. for 10 hours. Crude was purified with column chromatography to yield 338 mg of 24-2. Intermediate 24-2 was hydrogenated to 24-3 with LAH in THF solvent stirred at 0° C. for 2 hours. Intermediate 24-3 (222 mg) was recovered after work up. Intermediate 24-3 can be transformed into 24-4 by reacting it with manganese dioxide in chloroform.

### Synthesis 25: Synthesis of indolizine-5-carbaldehyde

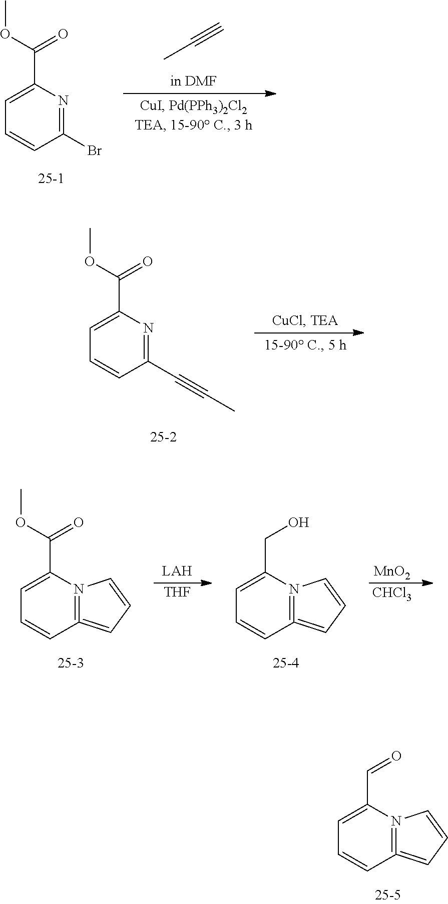

Methyl 6-bromopicolinate (25-1, 500 mg) was transformed into methyl 6-(prop-1-yn-1-yl)picolinate (25-2) by reacting it with 1-propyne dissolved in DMF in presence of copper iodide, bis(triphenylphosphine)palladium dichloride in triethylamine heated from 15° C. to 90° C. for 3 hours. Intermediate 25-2 (241.3 mg) was recovered after column chromatography purification. Intermediate 25-2 was then transformed into 25-3 with copper chloride in presence of triethylamine (TEA), by heating from 15° C. to 90° C. for 5 hours to afford 60 mg of material. Intermediate 25-3 was then hydrogenated to 25-4 by reacting it with LAH in THE solvent to afford 40 mg of material. Intermediate 25-4 can be further oxidized to 25-5 with manganese dioxide in chloroform.

### Synthesis 26: Synthesis of indolizine-5-carbaldehyde

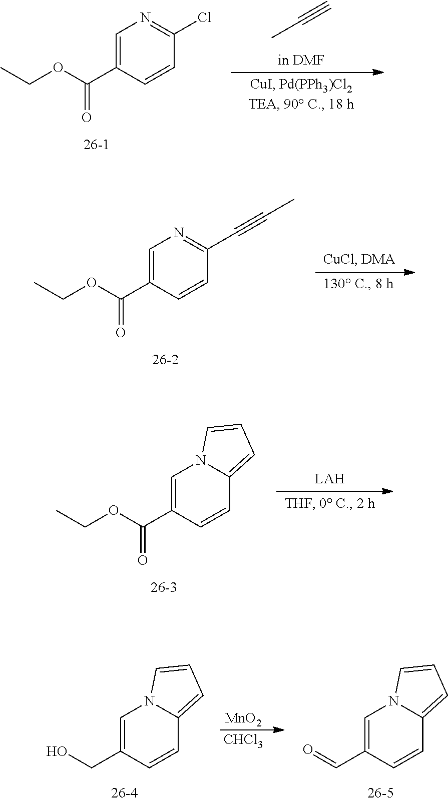

5 g of ethyl 6-chloronicotinate (26-1) was transformed into ethyl 6-(prop-1-yn-1-yl)nicotinate (26-2) by reacting it with 1-propyne dissolved in DMF in presence of copper iodide, bis(triphenylphosphine)palladium dichloride in triethylamine heated at 90° C. for 18 hours. 4.8 g of 26-2 was recovered after column chromatography purification. Intermediate 26-2 was transformed into 26-3 with copper chloride, in presence of dimethylamine (DMA), by heating at 130° C. for 8 hours. Intermediate 26-3 (1.5 g) was recovered after column chromatography purification. Intermediate 26-3 was hydrogenated to 26-4 by reacting it with LAH in THE solvent at 0° C. for 2 hours. Intermediate 26-4 can be further oxidized to 26-5 with manganese dioxide in chloroform.

### Synthesis 27: Synthesis of indolizine-7-carbaldehyde

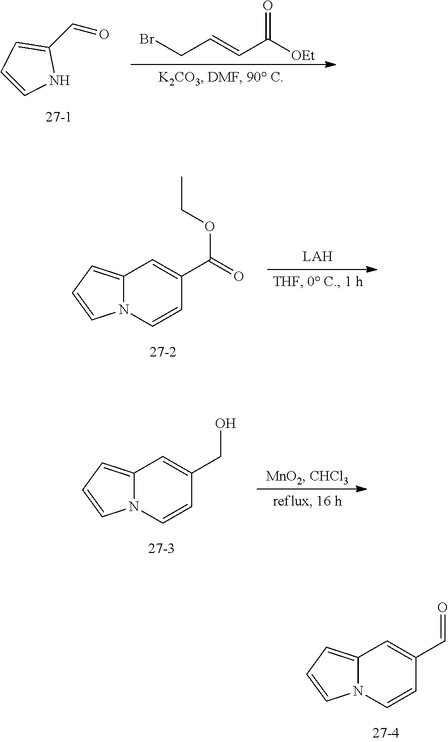

500 mg of 1H-pyrrole-2-carbaldehyde (27-1) was transformed into ethyl indolizine-7-carboxylate (27-2) by reacting it with ethyl (E)-4-bromobut-2-enoate in presence of potassium carbonate in dimethylformamide (DMF) solvent at 90° C. Intermediate 27-2 (390 mg) was recovered after column chromatography purification. Intermediate 27-2 was hydrogenated to 27-3 by reacting it with LAH in THF solvent at 0° C. for 1 hour. Intermediate 27-3 (100 mg) was transformed into 27-4 by reacting it with manganese dioxide in chloroform at reflux temperature for 16 hours. Intermediate 27-4 (10 mg) was recovered after column chromatography purification.

### Synthesis 28: Synthesis of indolizine-8-methylene chloride

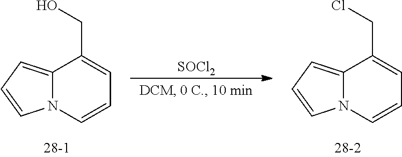 [SMILES: OCc1cccn2cccc12 ; "SOCl2" ; "DCM, 0 C., 10 min" ; ClCc1cccn2cccc12]

### Example 3: Synthesis of 1-(indolizin-1-yl)-N-methylalkane-2-amine

### Synthesis 29: Synthesis of Structure XLVI

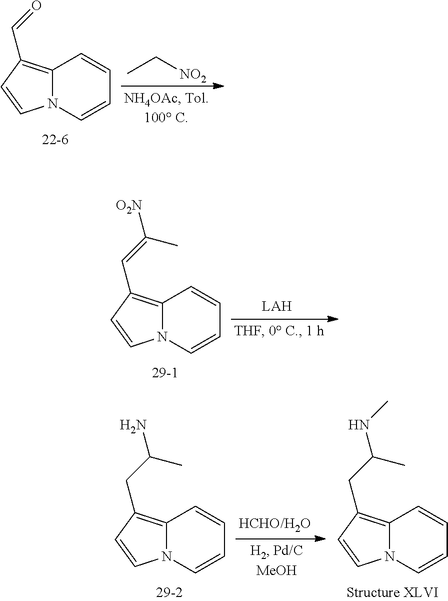

Indolizine-1-carbaldehyde (22-6) can be reacted with nitroethane in presence of ammonium acetate and toluene at 100° C. and can be transformed into 29-1. Intermediate 29-1 can be transformed into 29-2 by reacting it with lithium aluminum hydride (LAH) in THF at 0° C. for 1 hour. Intermediate 29-2 can be transformed into Structure XLVI by reacting with formaldehyde in water in presence of hydrogen gas and palladium over carbon in methanol.

### Synthesis 30: Synthesis of structures XLVIII and XLIX

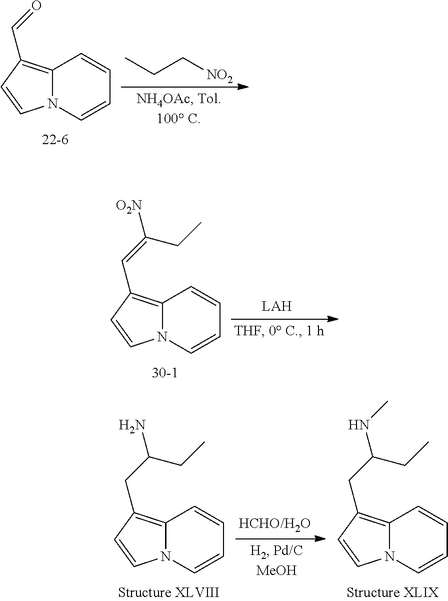

Indolizine-1-carbaldehyde (22-6) can be reacted with 1-nitropropane in presence of ammonium acetate and toluene at 100° C. and can be transformed into 30-1. Intermediate 30-1 can be transformed into Structure XLVIII by reacting it with lithium aluminum hydride (LAH) in THF at 0° C. for 1 hour. Structure XLVIII can be transformed into Structure XLIX by reacting with formaldehyde in water in presence of hydrogen gas and palladium over carbon in methanol.

### Example 4: Synthesis of 1-(indolizin-2-yl)-N-methylalkan-2-amine

### Synthesis 31: Synthesis of Structure II

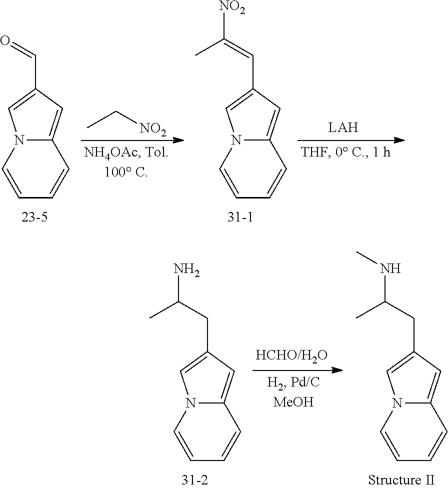

Indolizine-2-carbaldehyde (23-5) can be reacted with nitroethane in presence of ammonium acetate and toluene at 100° C. and can be transformed into 31-1. Intermediate 31-1 can be transformed into 31-2 by reacting it with lithium aluminum hydride (LAH) in THF at 0° C. for 1 hour. Intermediate 31-2 can be transformed into Structure II by reacting with formaldehyde in water in presence of hydrogen gas and palladium over carbon in methanol.

### Synthesis 32: Synthesis of structures IV and V

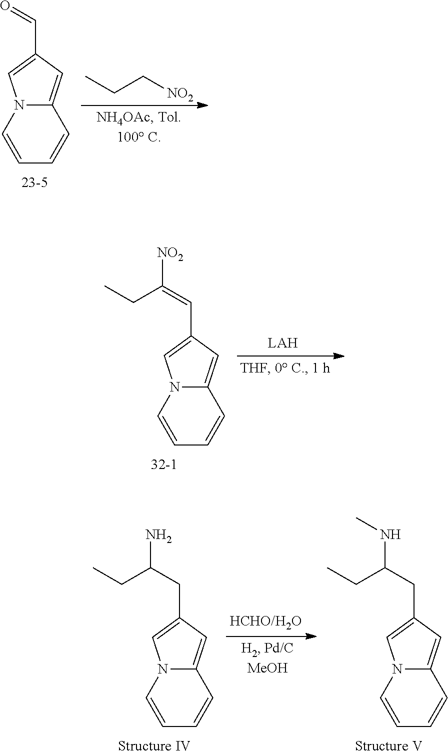

Indolizine-2-carbaldehyde (23-5) can be reacted with 1-nitropropane in presence of ammonium acetate and toluene at 100° C. and can be transformed into 32-1. Intermediate 32-1 can be transformed into Structure IV by reacting it with lithium aluminum hydride (LAH) in THF at 0° C. for 1 hour. Structure IV can be transformed into Structure V by reacting with formaldehyde in water in presence of hydrogen gas and palladium over carbon in methanol.

### Example 5: Synthesis of 1-(indolizin-3-yl)-N-methylalkan-2-amine

### Synthesis 33: Synthesis of Structure VII

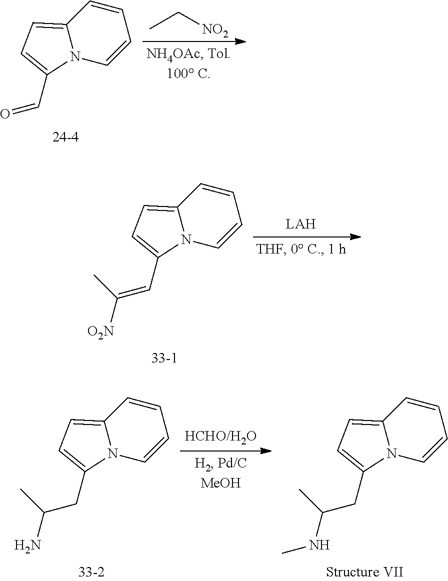

Indolizine-3-carbaldehyde (24-4) can be reacted with nitroethane in presence of ammonium acetate and toluene at 100° C. and can be transformed into 33-1. Intermediate 33-1 can be transformed into 33-2 by reacting it with lithium aluminum hydride (LAH) in THF at 0° C. for 1 hour. Intermediate 33-2 can be transformed into Structure VII by reacting with formaldehyde in water in presence of hydrogen gas and palladium over carbon in methanol.

### Synthesis 34: Synthesis of Structures IX and X

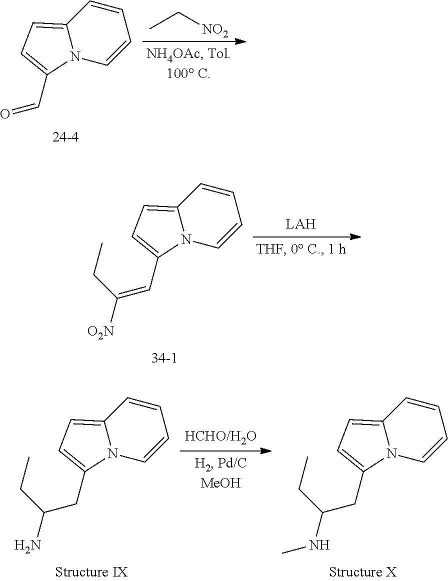

Indolizine-3-carbaldehyde (24-5) can be reacted with 1-nitropropane in presence of ammonium acetate and toluene at 100° C. and can be transformed into 34-1. Intermediate 34-1 can be transformed into Structure IX by reacting it with lithium aluminum hydride (LAH) in THF at 0° C. for 1 hour. Structure IX can be transformed into Structure X by reacting with formaldehyde in water in presence of hydrogen gas and palladium over carbon in methanol.

### Example 6: Synthesis of 1-(indolizin-5-yl)-N-methylalkan-2-amine

### Synthesis 35: Synthesis of Structure XIII

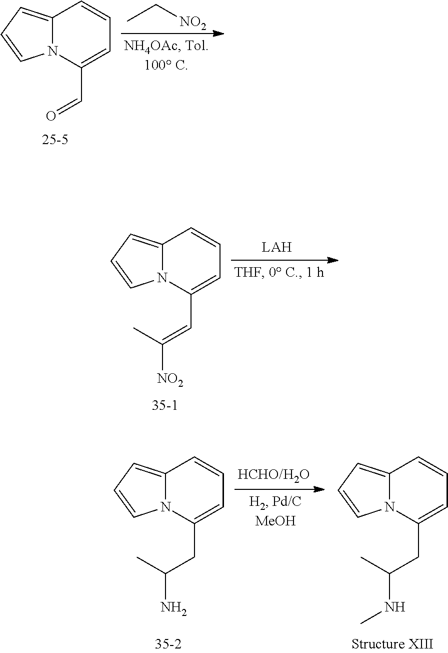

Indolizine-5-carbaldehyde (25-5) can be reacted with nitroethane in presence of ammonium acetate and toluene at 100° C. and can be transformed into 35-1. Intermediate 35-1 can be transformed into 35-2 by reacting it with lithium aluminum hydride (LAH) in THF at 0° C. for 1 hour. Intermediate 35-2 can be transformed into Structure XIII by reacting with formaldehyde in water in presence of hydrogen gas and palladium over carbon in methanol.

### Synthesis 36: Synthesis of Structures XV and XVI

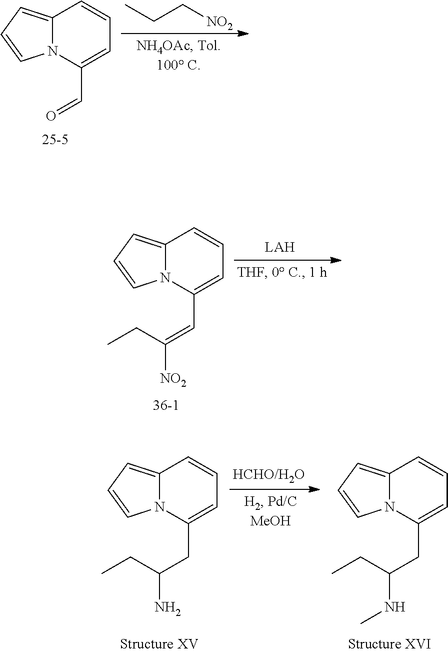

Indolizine-5-carbaldehyde (25-5) can be reacted with 1-nitropropane in presence of ammonium acetate and toluene at 100° C. and can be transformed into 36-1. Intermediate 36-1 can be transformed into Structure XV by reacting it with lithium aluminum hydride (LAH) in THF at 0° C. for 1 hour. Structure XV can be transformed into Structure XVI by reacting with formaldehyde in water in presence of hydrogen gas and palladium over carbon in methanol.

### Example 7: Synthesis of 1-(indolizin-6-yl)-N-methylalkan-2-amine

### Synthesis 37: Synthesis of Structure XXV

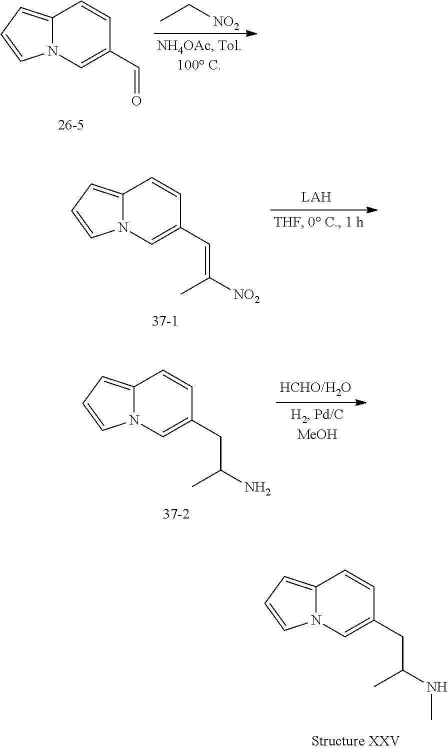

Indolizine-6-carbaldehyde (26-5) can be reacted with nitroethane in presence of ammonium acetate and toluene at 100° C. and can be transformed into 37-1. Intermediate 37-1 can be transformed into 37-2 by reacting it with lithium aluminum hydride (LAH) in THF at 0° C. for 1 hour. Intermediate 37-2 can be transformed into Structure XXV by reacting with formaldehyde in water in presence of hydrogen gas and palladium over carbon in methanol.

### Synthesis 38: Synthesis of Structures XXVII and XXVIII

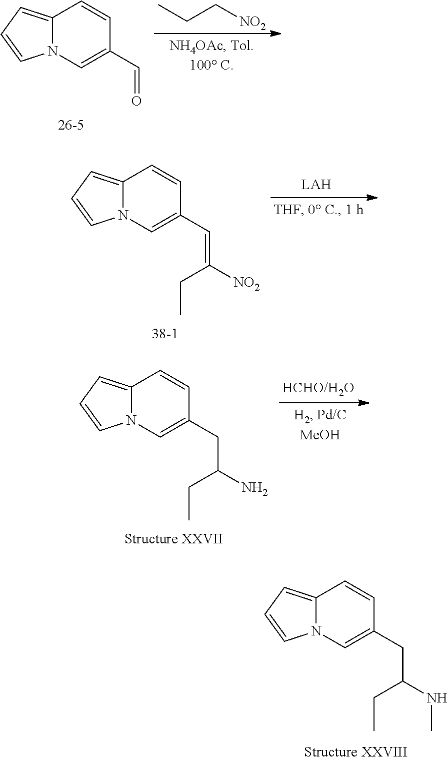

Indolizine-6-carbaldehyde (26-5) can be reacted with 1-nitropropane in presence of ammonium acetate and toluene at 100° C. and can be transformed into 38-1. Intermediate 38-1 can be transformed into Structure XXVII by reacting it with lithium aluminum hydride (LAH) in THF at 0° C. for 1 hour. Structure XXVII can be transformed into Structure XXVIII by reacting with formaldehyde in water in presence of hydrogen gas and palladium over carbon in methanol.

### Example 8: Synthesis of 1-(indolizin-7-yl)-N-methylalkan-2-amine

### Synthesis 39: Synthesis of Structure XXXI

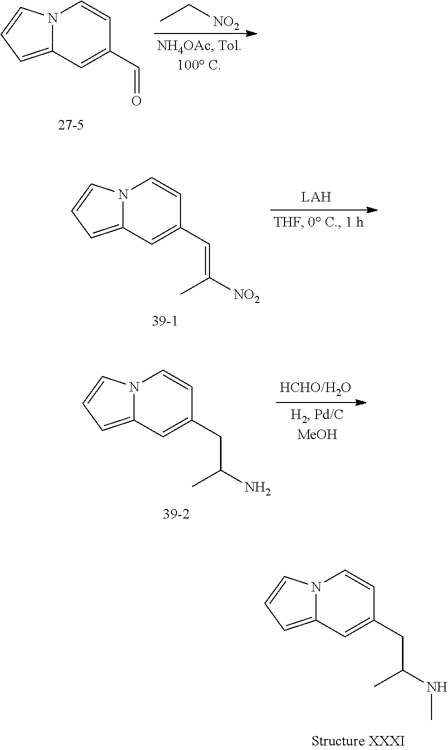

Indolizine-7-carbaldehyde (27-5) can be reacted with nitroethane in presence of ammonium acetate and toluene at 100° C. and can be transformed into 39-1. Intermediate 39-1 can be transformed into 39-2 by reacting it with lithium aluminum hydride (LAH) in THF at 0° C. for 1 hour. Intermediate 39-2 can be transformed into Structure XXXI by reacting with formaldehyde in water in presence of hydrogen gas and palladium over carbon in methanol.

### Synthesis 40: Synthesis of Structures XXXIII and XXXIV

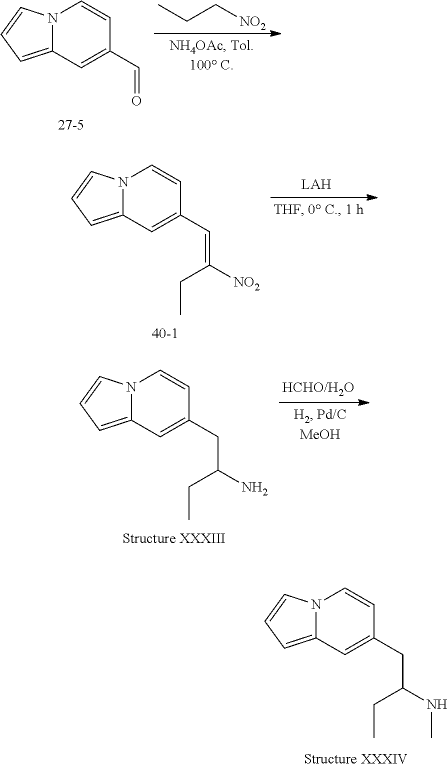

Indolizine-7-carbaldehyde (27-5) can be reacted with 1-nitropropane in presence of ammonium acetate and toluene at 100° C. and can be transformed into 40-1. Intermediate 40-1 can be transformed into Structure XXXIII by reacting it with lithium aluminum hydride (LAH) in THE at 0° C. for 1 hour. Structure XXXIII can be transformed into Structure XXXIV by reacting with formaldehyde in water in presence of hydrogen gas and palladium over carbon in methanol.

### Example 9: Synthesis of 1-(indolizin-8-yl)-N-methylalkan-2-amine

### Synthesis 41: Synthesis of Structure XIX, XXI, and XXII

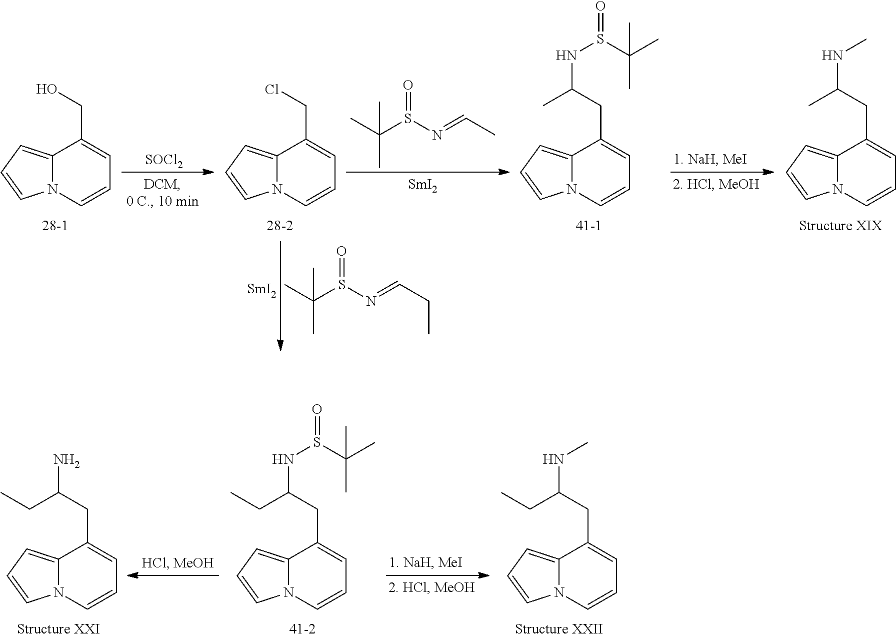

### Example 10: Evaluation of Therapeutic Properties

The clinical and therapeutic effects of compounds that increase extracellular monoamine neurotransmitters are thought to be correlated with their relative tendencies to increase serotonin and dopamine. Liechti and colleagues have proposed that new psychoactive drugs can be classified based on their DAT/SERT inhibition ratios, defined as 1/IC₅₀ at DAT divided by 1/IC₅₀ at SERT (e.g., Luethi and Liechti. 2020. Archives of toxicology, 94(4), pp. 1085-1133). These authors use IC₅₀ measuring uptake inhibition rather than EC₅₀ measuring neurotransmitter release, presumably because drugs that release neurotransmitter also have measurable effects in uptake inhibition assays, producing a metric that can accommodate both reuptake inhibitors and releasers.

In the classification system of Liechti and colleagues, DAT/SERT IC₅₀ ratios>1 are thought to predict psychostimulant effects and compounds with this profile have potential value in treating attention deficit hyperactivity disorder (ADHD) and stimulant use disorders. Example compounds with this profile include dextroamphetamine and methylphenidate (Ritalin, Concerta).

In contrast, serotonin release and a DAT/SERT IC₅₀ ratio of 0.01-0.1 is said to result in a psychoactive drug profile similar to that of MDMA, which includes feelings of emotional openness, authenticity, and decreased neuroticism. MDMA is an experimental adjunct to psychotherapy that shows great potential for treating PTSD and substance use disorders. It may also be able to generally accelerate progress in psychotherapy and aid emotional decision making. MDMA has a reported DAT/SERT IC₅₀ ratio of 0.08 (Simmler and Liechti, New Psychoactive Substances, pp. 143-164).

Compounds with intermediate DAT/SERT IC₅₀ ratios (between 0.1 and 1) appear to sometimes have antidepressant-like or nootropic (cognitive enhancement) qualities and have been proposed as antidepressants, cognitive enhancers, or treatments for substance use disorders. For example, 4-bromomethcathinone (4-BMC, Brephedrone; IUPAC: 1-(4-bromophenyl)-2-(methylamino)propan-1-one) does not have typical psychostimulant effects and has been proposed as a potential antidepressant (Foley and Cozzi. 2003. Drug development research, 60(4), pp. 252-260). The different therapeutic profiles of these intermediate compounds are believed to be at least partially the result of serotonin inhibiting and modifying the stimulating effects of dopamine (Kimmel et al. 2009. Pharmacology Biochemistry and Behavior, 94(2), pp. 278-284; Suyama et al. 2019. Psychopharmacology, 236(3), pp. 1057-1066; Wee et al. 2005. Journal of Pharmacology and Experimental Therapeutics, 313(2), pp. 848-854).

One caveat to Liechti's classification system is that compounds that release neurotransmitter may be misclassified if their relative abilities to release dopamine and serotonin are substantially different from their relative abilities to inhibit uptake of dopamine and serotonin. Compounds that appear misclassified in this manner include 3,4,-methylenedioxyethylamphetamine (MDEA; IUPAC [1-(2H-1,3-benzodioxol-5-yl)propan-2-yl](ethyl)amine), which has a reported DAT/SERT IC₅₀ ratio of 3.2 (Simmler et al. 2013. British journal of pharmacology, 168(2), pp. 458-470) but is also reported to have MDMA-like effects in humans (e.g., Hermle et al. 1993. Neuropsychopharmacology, 8(2), pp. 171-176).

Such releasing compounds may be alternatively classified according to their DAT/SERT EC₅₀ ratios, where MDEA has been reported as 0.76 (Rothman et al. 2012. Journal of Pharmacology and Experimental Therapeutics, 341(1), pp. 251-262). In this release-based system, MDMA-like therapeutic effects appear present at ratios below 2, with compounds having DAT/SERT EC₅₀ ratios between 2 and 5 having diminished but often still noticeable MDMA-like effects. These intermediate compounds may prove useful for treating ADHD, substance use disorders, and other conditions in individuals who experience significant anxiety from approved psychostimulant pharmacotherapies such as d-amphetamine. Similar to the IC₅₀ system, compounds with higher DAT/SERT EC₅₀ ratios are potential treatments for ADHD and psychostimulant use disorders.

Although MDMA has significant therapeutic potential, it has a number of features that limit its clinical use and may make it contraindicated for some patients. This includes its moderate abuse liability (likely related to its ability to increase extracellular dopamine), acute hypertensive effects (likely related to its norepinephrine release), variable inter-individual metabolism that includes inhibition of the liver enzyme CYP2D6 (increasing risk of drug-drug interactions), potential to induce hyponatremia in women, oxidative stress (likely related to its extensive, though variable, metabolism and formation of reactive metabolites), ability to produce decreases in SERT density after high doses, diminishing therapeutic benefits with repeated use; and a hangover-like after-effects including poor mood and lowered energy. There is therefore a need for additional pharmacologic agents that have similar therapeutic properties while having different pharmacological profiles compared to MDMA.

Compounds that increase extracellular dopamine also often increase extracellular norepinephrine to a similar or greatest extent. For example, d-methamphetamine has a reported DAT/NET EC₅₀ ratio of 0.5, while d-amphetamine has a ratio of 0.9 (Rothman et al. 2001. Synapse, 39(1), pp. 32-41), indicating both are more potent at increasing norepinephrine than dopamine. Differences in the relative balance of dopamine and norepinephrine increases can yield compounds with valuable therapeutic profiles. Norepinephrine increases contribute to cognitive improvements in ADHD but, in excess, can also lead to cardiovascular changes. Dopamine similarly modulates impulsive action but, in excess, can produce compounds with high abuse liability. Nonetheless, individuals with histories of substance abuse who have desensitized dopamine receptors can benefit from compounds that adequately stimulate these receptors. Thus, there is a need for novel treatment compounds that differently balance therapeutic benefits against cardiovascular and abuse liability side effects.

As previously noted, increases in extracellular serotonin and direct stimulation of serotonin receptors present ways for compounds (or compound combinations) to decrease off-target effects and increase select therapeutic effects. For example, compounds that release dopamine and/or norepinephrine and also stimulate 5-HT_{1A} or 5-HT_{1B} receptors can provide fast acting therapeutic effects on mood and attention while decreasing social anxiety. Similarly, compounds that stimulate 5-HT_{2A} receptors while increasing extracellular neurotransmitter can provide the therapeutic benefits of 5-HT_{2A} agonists while having predictable positive effects on mood that decrease the need for clinical monitoring.

### Example 11: In Vitro Activity Assessments

1-(indolizin-3-yl)-2-(methylamino)propan-1-one (Structure XLX) was evaluated for activity at 47 target sites at ten concentrations up to 30 μM, with EC50 or IC50 determined whenever possible. Activity was detected only at 5-HT_{1B} (agonist effects with an EC50 of 1.13 μM) and 5-HT2B (antagonist effects with an IC50 of 2.14 μM) receptors and at DAT (IC50 of 2.41 μM) and NET (IC50 of 5.69 μM). Concentrations of test compound was about 0.00152416, 0.0045724, 0.0137174, 0.041152, 0.123456, 0.37038, 1.11112, 3.3334, 10, and 30 μM.

### Assay-1: CAMP Secondary Messenger Assay

CAMP secondary messenger assays used cell lines that stably expressed non-tagged GPCRs. Hit Hunter® CAMP assays monitored the activation of a GPCR via Gi and Gs secondary messenger signaling in a homogenous, non-imaging assay format using Enzyme Fragment Complementation (EFC) with β-galactosidase (β-gal) as the functional endpoint. For the assay system, exogenously introduced Enzyme Donor (ED) fused to cAMP (ED-cAMP) competes with endogenously generated cAMP for binding to an anti-cAMP-specific antibody. Active β-gal is formed by complementation of exogenous Enzyme Acceptor (EA) to any unbound ED-cAMP. Active enzyme can then convert a chemiluminescent substrate, generating an output signal detectable on a standard microplate reader.

Assay Design: GPCR cAMP Modulation

### Cell Handling

1. cAMP Hunter cell lines were expanded from freezer stocks according to standard procedures.
2. Cells were seeded in a total volume of 20 μL into white walled, 384-well microplates and incubated at 37° C. for the appropriate time prior to testing.
3. cAMP modulation was determined using the DiscoverX HitHunter cAMP XS+ assay.

### Gs Agonist Format

1. For agonist determination, cells were incubated with sample to induce response.
2. Media was aspirated from cells and replaced with 15 μL 2:1 HBSS/10 mM Hepes: cAMP XS+ Ab reagent.
3. Intermediate dilution of sample stocks was performed to generate 4× sample in assay buffer.
4. 5 μL of 4× sample was added to cells and incubated at 37° C. or room temperature for 30 or 60 minutes.

### Gi Agonist Format

1. For agonist determination, cells were incubated with sample in the presence of EC₈₀ forskolin to induce response.
2. Media was aspirated from cells and replaced with 15 μL 2:1 HBSS/10MM Hepes: cAMP XS+Ab reagent.
3. Intermediate dilution of sample stocks was performed to generate 4× sample in assay buffer containing 4×EC₅₀ forskolin.
4. 5 μL of 4× sample was added to cells and incubated at 37° C. or room temperature for 30 or 60 minutes.

### Antagonist Format

1. For antagonist determination, cells were pre-incubated with sample followed by agonist challenge at the EC80 concentration.
2. Media was aspirated from cells and replaced with 10 μL 1:1 HBSS/Hepes: cAMP XS+Ab reagent.
3. 5 μL of 4× compound was added to the cells and incubated at 37° C. or room temperature for 30 minutes.
4. 5 μL of 4×EC₅₀ agonist was added to cells and incubated at 37° C. or room temperature for 30 or 60 minutes. For Gi coupled GPCRs, EC₅₀ forskolin was included.

### Signal Detection

1. After appropriate compound incubation, assay signal was generated through incubation with 20 μL cAMP XS+ED/CL lysis cocktail for one hour followed by incubation with 20 μL cAMP XS+EA reagent for three hours at room temperature.
2. Microplates were read following signal generation with PerkinElmer Envision instrument for chemiluminescent signal detection.

### Data Analysis

1. Compound activity was analyzed using CBIS data analysis suite (ChemInnovation, CA).
2. For Gs agonist mode assays, percentage activity was calculated using the following formula: % Activity=100%×(mean RLU of test sample−mean RLU of vehicle control)/(mean RLU of MAX control−mean RLU of vehicle control).
3. For Gs antagonist mode assays, percentage inhibition was calculated using the following formula: % Inhibition=100%×(1−(mean RLU of test sample−mean RLU of vehicle control)/(mean RLU of EC₅₀ control−mean RLU of vehicle control)).
4. For Gi agonist mode assays, percentage activity was calculated using the following formula: % Activity=100%×(1−(mean RLU of test sample−mean RLU of MAX control)/(mean RLU of vehicle control−mean RLU of MAX control)).
5. For Gi antagonist or negative allosteric mode assays, percentage inhibition was calculated using the following formula: % Inhibition=100%×(mean RLU of test sample−mean RLU of EC₈₀ control)/(mean RLU of forskolin positive control−mean RLU of EC₈₀ control).

For screening, percent response was capped at 0% or 100% where calculated percent response returned a negative value or a value greater than 100, respectively.

### Assay-2: Calcium Flux Secondary Messenger Assay

The Calcium No Wash^{PLUS} assay was used to monitor GPCR activity via Gq secondary messenger signaling in a live cell, non-imaging assay format. Calcium mobilization in PathHunter® cell lines or other cell lines stably expressing Gq-coupled GPCRs was monitored using calcium-sensitive dye loaded into cells. GPCR activation by a compound resulted in the release of calcium from intracellular stores and an increase in dye fluorescence that was measured in real-time.

### Assay Design: Calcium Mobilization

### Cell Handling

1. Cell lines were expanded from freezer stocks according to standard procedures.
2. Cells (10,000 cells/well) were seeded in a total volume of 50 μL (200 cells/μL) into black-walled, clear-bottom, Poly-D-lysine coated 384-well microplates and incubated at 37° C. for the appropriate time prior to testing.

### Dye Loading

1. Assays were performed in 1× Dye Loading Buffer consisting of 1× Dye (DiscoverX, Calcium No Wash PLUS kit, Catalog No. 90-0091), 1× Additive A and 2.5 mM Probenecid in HBSS/20 mM Hepes. Probenecid was prepared fresh.
2. Cells were loaded with dye prior to testing. Media was aspirated from cells and replaced with 25 μL Dye Loading Buffer.
3. Cells were incubated for 45 minutes at 37° C. and then 20 minutes at room temperature.

### Agonist Format

1. For agonist determination, cells were incubated with sample to induce response.
2. After dye loading, cells were removed from the incubator and 25 μL of 2× compound in HBSS/20 mM Hepes was added using a FLIPR Tetra (MDS).
3. Compound agonist activity was measured on a FLIPR Tetra. Calcium mobilization was monitored for 2 minutes with a 5 second baseline read.

### Antagonist Format

1. For antagonist determination, cells were pre-incubated with sample followed by agonist challenge at the EC₅₀ concentration.
2. After dye loading, cells were removed from the incubator and 25 μL 2× sample was added. Cells were incubated for 30 minutes at room temperature in the dark to equilibrate plate temperature.
3. After incubation, antagonist determination was initiated with addition of 25 μL 1× compound with 3×EC₅₀ agonist using FLIPR
4. Compound antagonist activity was measured on a FLIPR Tetra (MDS). Calcium mobilization was monitored for 2 minutes with a 5 second baseline read.

### Data Analysis

1. FLIPR read—Area under the curve was calculated for the entire two-minute read.
2. Compound activity was analyzed using CBIS data analysis suite (ChemInnovation, CA).
3. For agonist mode assays, percentage activity was calculated as: % Activity=100%×(mean RFU of test sample−mean RFU of vehicle control)/(mean MAX RFU control ligand−mean RFU of vehicle control).
4. For antagonist mode assays, percentage inhibition was calculated as: % Inhibition=100%×(1−(mean RFU of test sample−mean RFU of vehicle control)/(mean RFU of EC80 control−mean RFU of vehicle control)).

For Primary screens, percent response was capped at 0% or 100% where calculated percent response returned a negative value or a value greater than 100, respectively.

### Assay-3: Nuclear Hormone Receptor Assay

PathHunter® NHR Protein Interaction (NHR Pro) and Nuclear Translocation (NHR NT) assays monitored the activation of specific nuclear hormone receptors in a homogenous, non-imaging assay format using Enzyme Fragment Complementation (EFC). The NHR Pro assay is based on detection of protein-protein interactions between an activated, full length NHR protein and a nuclear fusion protein containing Steroid Receptor Co-activator Peptide (SRCP) domains with one or more canonical LXXLL interaction motifs. The NHR was tagged with the ProLink™ (PK) component of the DiscoverX EFC assay system, and the SRCP domain was fused to the Enzyme Acceptor component (EA) expressed in the nucleus. When bound by ligand, the NHR migrates to the nucleus and recruites the SRCP domain, whereby complementation occurs, generating a unit of active β-galactosidase (β-gal) and production of chemiluminescent signal upon the addition of PathHunter detection reagents. The NHR NT assay monitored movement of an NHR between the cytoplasmic and nuclear compartments. The receptor was tagged with the ProLabel™ (PL) component of the EFC assay system, and EA was fused to a nuclear location sequence that restricted the expression of EA to the nucleus. Migration of the NHR to the nucleus resulted in complementation with EA generating a unit of active β-gal and production of a chemiluminescent signal upon the addition of Path Hunter detection reagents.

### Assay Design: Nuclear Hormone Receptor

### Cell Handling

1. PathHunter NHR cell lines were expanded from freezer stocks according to standard procedures.
2. Cells were seeded in a total volume of 20 μL into white walled, 384-well microplates and incubated at 37° C. for the appropriate time prior to testing. Assay media contained charcoal-dextran filtered serum to reduce the level of hormones present.

### Agonist Format

1. For agonist determination, cells were incubated with sample to induce response.
2. Intermediate dilution of sample stocks was performed to generate 5× sample in assay buffer.
3. 5 μL of 5× sample was added to cells and incubated at 37° C. or room temperature for 3-16 hours.

### Antagonist Format

1. For antagonist determination, cells were pre-incubated with antagonist followed by agonist challenge at the EC₅₀ concentration.
2. Intermediate dilution of sample stocks was performed to generate 5× sample in assay buffer.
3. 5 μL of 5× sample was added to cells and incubated at 37° C. or room temperature for 60 minutes. Vehicle concentration was 1%.
4. 5 μL of 6×EC80 agonist in assay buffer was added to the cells and incubated at 37° C. or room temperature for 3-16 hours.

### Signal Detection

1. Assay signal was generated through a single addition of 12.5 or 15 μL (50% v/v) of PathHunter Detection reagent cocktail, followed by a one-hour incubation at room temperature.
2. Microplates were read following signal generation with a PerkinElmer Envision instrument for chemiluminescent signal detection.

### Data Analysis

1. Compound activity was analyzed using CBIS data analysis suite (ChemInnovation, CA).
2. For agonist mode assays, percentage activity was calculated as: % Activity=100%×(mean RLU of test sample−mean RLU of vehicle control)/(mean MAX control ligand −mean RLU of vehicle control).
3. For antagonist mode assays, percentage inhibition was calculated as: % Inhibition=100%×(1−(mean RLU of test sample−mean RLU of vehicle control)/(mean RLU of EC80 control−mean RLU of vehicle control)).
4. Note that for select assays, the ligand response produces a decrease in receptor activity (inverse agonist with a constitutively active target). For those assays inverse agonist activity was calculated as: % Inverse Agonist Activity=100%×((mean RLU of vehicle control−mean RLU of test sample)/(mean RLU of vehicle control−mean RLU of MAX control)).

For Primary screens, percent response was capped at 0% or 100% where calculated percent response returned a negative value or a value greater than 100, respectively.

### Assay-4: KINOMEscan® Assay

Kinase activity was measured using the KINOMEscan screening platform, which employs a site-directed competition binding assay to quantitatively measure interactions between test compounds and the kinases. Compounds that bind the kinase active site and directly (sterically) or indirectly (allosterically) prevent kinase binding to the immobilized ligand, will reduce the amount of kinase captured on the solid support (A and B). Conversely, test molecules that do not bind the kinase have no effect on the amount of kinase captured on the solid support (C). Screening “hits” were identified by measuring the amount of kinase captured in test versus control samples by using a quantitative, precise and ultra-sensitive qPCR method that detects the associated DNA label (D). In a similar manner, dissociation constants (Kds) for test compound-kinase interactions were calculated by measuring the amount of kinase captured on the solid support as a function of the test compound concentration.

Assay Design: KINOME scan Binding Assays

### Protein Expression

For most assays, kinase-tagged T7 phage strains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (6,000×g) and filtered (0.2 m) to remove cell debris. The remaining kinases were produced in HEK-293 cells and subsequently tagged with DNA for qPCR detection.

### Capture Ligand Production

Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding.

### Binding Reaction Assembly

Binding reactions were assembled by combining kinases, liganded affinity beads, and test compounds in 1× binding buffer (20% SeaBlock, 0.17×PBS, 0.05% Tween 20, 6 mM DTT). All reactions were performed in polypropylene 384-well plates in a final volume of 0.02 mL. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The kinase concentration in the eluates was measured by qPCR.

### Signal Detection

The kinase concentration in the eluates was measured by qPCR. qPCR reactions were assembled by adding 2.5 μL of kinase eluate to 7.5 μL of qPCR master mix containing 0.15 μM amplicon primers and 0.15 μM amplicon probe. The qPCR protocol consisted of a 10-minute hot start at 950 C, followed by 35 cycles of 95° C. for 15 seconds, 60° C. for 1 minute.

### Data Analysis

### Percent Response Calculation

100*(test compound signal−positive control signal)/(negative compound signal−positive control signal)
where
   Test compound=compound submitted by Customer
   Negative control=DMSO (100% Ctrl)
   Positive control=control compound (0% Ctrl)
Percent of Control was converted to Percent Response with the conversion: Percent Response=(100−Percent Control).

For Primary screens, percent response was capped at 0% or 100% where calculated percent response returned a negative value or a value greater than 100, respectively.

### Binding Constants (Kds)

Binding constants (Kds) were calculated with a standard dose response curve using the Hill equation with Hill Slope set to −1.

Curves were fitted using a non-linear least square fit with the Levenberg-Marquardt algorithm.

### Assay-5: Monoamine Transporter Uptake Assay

The Neurotransmitter Transporter Uptake Assay Kit from Molecular Devices was used as a homogeneous fluorescence-based assay for the detection of dopamine, norepinephrine or serotonin transporter activity in cells expressing these transporters. The kit employs a fluorescent substrate that mimics the biogenic amine neurotransmitters that are taken into the cell through these specific transporters, resulting in increased intracellular fluorescence intensity. It should be noted that fluorescence-based assays for the detection of dopamine, norepinephrine or serotonin transporter activity have poor sensitivity for compounds that are substrates for these monoamine transporters. We therefore separately measured interactions with these transporters using two additional types of assays: an antagonist radioligand assay of inhibition of the human 5-HT transporter (hSERT) expressed in CHO cells (Tatsumi, M. et al. (1999), Eur. J. Pharmacol., 368: 277-283) and an assay measuring release of [³H] Serotonin or [³H] dopamine, respectively, from cells stably expressing SERT or DAT. While the former is sensitive to classic reuptake inhibition, the latter can detect the effects of substrates, which also induce release.

### Assay Design: Transporter Assays

### Cell Handling

1. Cell lines were expanded from freezer stocks according to standard procedures.
2. Cells were seeded in a total volume of 25 μL into black-walled, clear-bottom, Poly-D-lysine coated 384-well microplates and incubated at 37° C. for the appropriate time prior to testing.

### Blocker/Antagonist Format

1. After cell plating and incubation, media was removed and 25 μL of 1× compound in 1×HBSS/0.1% BSA was added.
2. Compounds were incubated with cells at 37° C. for 30 minutes.

### Dye Loading

1. Assays were performed in 1× Dye Loading Buffer consisting of 1× Dye, 1×HBSS/20 mM Hepes.
2. After compound incubation, 25 μL of 1× dye was added to wells.
3. Cells were incubated for 30-60 minutes at 37° C.

### Signal Detection

1. After dye incubation, microplates were transferred to a PerkinElmer Envision instrument for fluorescence signal detection.

### Data Analysis

1. Compound activity was analyzed using CBIS data analysis suite (ChemInnovation, CA).
2. For blocker mode assays, percentage inhibition was calculated using the following formula: % Inhibition=100%×(1−(mean RLU of test sample−mean RLU of vehicle control)/(mean RLU of positive control−mean RLU of vehicle control)).

For Primary screens, percent response was capped at 0% or 100% where calculated percent response returned a negative value or a value greater than 100, respectively.

### Assay-6: Potassium Assay

The FLIPR Potassium Assay Kit from Molecular Devices was used for ion channel assays. This approach exploited the permeability of thallium ions (TI+) through both voltage and ligand-gated potassium (K+) channels. A highly-sensitive Tl+ indicator dye produced a bright fluorescent signal upon the binding to Tl+ conducted through potassium channels. The intensity of the Tl+ signal was proportional to the number of potassium channels in the open state and therefore provided a functional indication of the potassium channel activities. In addition, a masking dye was included to reduce background fluorescence for improved signal/noise ratio.

Specific assay steps and reference compounds are given below.

### Assay-7: Membrane Potential Assay

The FLIPR® Membrane Potential Assay Kit was used which employs a fluorescent indicator dye in combination with a quencher to reflect real-time membrane potential changes associated with ion channel activation and ion transporter proteins. Unlike traditional dyes such as DiBAC, the FLIPR Membrane Potential Assay Kit detects bidirectional ion fluxes so both variable and control conditions can be monitored within a single experiment.

Specific assay steps and reference compounds are given below.

### Assay-8: Calcium Assay

The DiscoveRx Calcium NWPLUS Assay Kit was used for detection of changes in intracellular calcium. Cells expressing a receptor of interest that signals through calcium were preloaded with a calcium sensitive dye and then treated with compound. Upon stimulation, the receptor signaled release of intracellular calcium, which resulted in an increase of dye fluorescence. Signal was measured on a fluorescent plate reader equipped with fluidic handling capable of detecting rapid changes in fluorescence upon compound stimulation.

Specific assay steps and reference compounds are given below.

### Assay Design: Ion Channel Assays

### Cell Handling

1. Cell lines were expanded from freezer stocks according to standard procedures.
2. Cells were seeded in a total volume of 20 μL into black-walled, clear-bottom, Poly-D-lysine coated 384-well microplates and incubated at 37° C. for the appropriate time prior to testing.

### Dye Loading

1. Assays were performed in 1× Dye Loading Buffer consisting of 1× Dye, and 2.5 mM Probenecid when applicable. Probenecid was prepared fresh.
2. Cells were loaded with dye prior to testing.
3. Cells were incubated for 30-60 minutes at 37° C.

### Agonist/Opener Format

1. For agonist determination, cells were incubated with sample to induce response.
2. Intermediate dilution of sample stocks was performed to generate 2-5× sample in assay buffer.
3. 10-25 μL of 2-5× sample was added to cells and incubated at 37° C. or room temperature for 30 minutes.

### Antagonist/Blocker Format

1. For antagonist determination, cells were pre-incubated with sample.
2. Intermediate dilution of sample stocks was performed to generate 2-5× sample in assay buffer.
3. After dye loading, cells were removed from the incubator and 10-25 μL 2-5× sample was added to cells in the presence of EC80 agonist when appropriate. Cells were incubated for 30 minutes at room temperature in the dark to equilibrate plate temperature.

### Signal Detection

1. Compound activity was measured on a FLIPR Tetra (MDS).

### Data Analysis

1. Compound activity was analyzed using CBIS data analysis suite (ChemInnovation, CA).
2. For agonist mode assays, percentage activity was calculated using the following formula: % Activity=100%×(mean RLU of test sample−mean RLU of vehicle control)/(mean MAX control ligand −mean RLU of vehicle control).
3. For antagonist percentage inhibition was calculated using the following formula: % Inhibition=100%×(1−(mean RLU of test sample−mean RLU of vehicle control)/(mean RLU of EC80 control−mean RLU of vehicle control)).

For Primary screens, percent response was capped at 0% or 100% where calculated percent response returned a negative value or a value greater than 100, respectively.

### Assay-9: Enzymatic Assays

Enzymatic assays determined enzymatic activity by measuring either the consumption of substrate or production of product over time. Different detection methods were used in each enzymatic assay to measure the concentrations of substrates and products, including spectrophotometric, fluorometric, and luminescent readouts.

### Assay Design: Enzymatic Assays

### Enzyme Preparations

Enzyme preparations were sourced from AChE (R&D Systems), COX1 and COX2 (BPS Bioscience), MAOA (Sigma), PDE3A and PDE4D2 (Signal Chem).

### Enzyme Activity Assays

1. Enzymatic assays determine the enzymatic activity by measuring either the consumption of substrate or production of product over time. Different detection methods were used in each enzymatic assay to measure the concentrations of value greater than 100, respectively. substrates and products.
2. ACHE: Enzyme and test compound were preincubated for 15 minutes at room temp before substrate addition. Acetylthiocholine and DTNB were added and incubated at room temperature for 30 minutes. Signal was detected by measuring absorbance at 405 nm. 3. COX1 & COX2: Enzyme stocks were diluted in Assay Buffer (40 mM Tris-HCl, 1×PBS, 0.5 mM Phenol, 0.01% Tween-20+100 nM Hematin) and allowed to equilibrate with compounds at room temperature for 30 minutes (binding incubation). Arachidonic acid (1.7 μM) and Ampliflu Red (2.5 μM) were prepared and dispensed into a reaction plate. Plates were read immediately on a fluorimeter with the emission detection at 590 nm and excitation wavelength 544 nm.
4. MAOA: Enzyme and test compound were preincubated for 15 minutes at 37° C. before substrate addition. The reaction was initiated by addition of kynuramine and incubated at 37° C. for 30 minutes. The reaction was terminated by addition of NaOH. The amount of 4-hydroquinoline formed was determined through spectrofluorometric readout with the emission detection at 380 nm and excitation wavelength 310 nm.
5. PDE3A & PDE4D2: Enzyme and test compound were preincubated for 15 minutes at room temp before substrate addition. cAMP substrate (at a concentration equal to EC₅₀) was added and incubated at room temperature for 30 minutes. Enzyme reaction was terminated by addition of 9 mM IBMX. Signal was detected using the HitHunter® cAMP detection kit.

### Signal Detection

1. For each assay, microplates were transferred to a PerkinElmer Envision instrument and readout as described.

### Data Analysis

1. Compound activity was analyzed using CBIS data analysis suite (ChemInnovation, CA).
2. For enzyme activity assays, percentage inhibition was calculated using the following formula: % Inhibition=100%×(1−(mean RLU of test sample−mean RLU of vehicle control)/(mean RLU of positive control−mean RLU of vehicle control)).

For Primary screens, percent response was capped at 0% or 100% where calculated percent response returned a negative value or a value greater than 100, respectively.

TABLE 3
Target Names and Reference Compound Activity (Positive Controls) in Assays

| | | | | Result | RC50 |
|---|---|---|---|---|---|
| Target | Abbreviation | Mode | Reference Compound | Type | (μM) |
| 5-Hydroxytryptamine | 5-HTR1A | Agonist | Serotonin Hydrochloride | EC50 | 0.00395 |
| (Serotonin) Receptor 1A | | | | | |
| 5-Hydroxytryptamine | 5-HTR1A | Antagonist | Spiperone | IC50 | 0.10535 |
| (Serotonin) Receptor 1A | | | | | |
| 5-Hydroxytryptamine | 5-HTR1B | Agonist | Serotonin Hydrochloride | EC50 | 2.00E−04 |
| (Serotonin) Receptor 1B | | | | | |
| 5-Hydroxytryptamine | 5-HTR1B | Antagonist | SB 224289 | IC50 | 0.00606 |
| (Serotonin) Receptor 1B | | | | | |
| 5-Hydroxytryptamine | 5-HTR2A | Agonist | Serotonin Hydrochloride | EC50 | 0.00257 |
| (Serotonin) Receptor 2A | | | | | |
| 5-Hydroxytryptamine | 5-HTR2A | Antagonist | Altanserin | IC50 | 0.01553 |
| (Serotonin) Receptor 2A | | | | | |
| 5-Hydroxytryptamine | 5-HTR2B | Agonist | Serotonin Hydrochloride | EC50 | 0.00396 |
| (Serotonin) Receptor 2B | | | | | |
| 5-Hydroxytryptamine | 5-HTR2B | Antagonist | LY 272015 | IC50 | 3.00E−04 |
| (Serotonin) Receptor 2B | | | | | |
| 5-Hydroxytryptamine | 5-HTR3A | Blocker | Bemesetron | IC50 | 0.00305 |
| (Serotonin) Receptor 3A | | | | | |
| 5-Hydroxytryptamine | 5-HTR3A | Opener | Serotonin Hydrochloride | EC50 | 0.36698 |
| (Serotonin) Receptor 3A | | | | | |
| Acetylcholinesterase | ACHE | Inhibitor | Physostigmine | IC50 | 0.03747 |
| Adenosine Receptor A2A | ADORA2A | Antagonist | SCH 442416 | IC50 | 0.0798 |
| Adenosine Receptor A2A | ADORA2A | Agonist | NECA | EC50 | 0.01783 |
| Adrenergic Receptor α₁A | ADRA1A | Agonist | A 61603 Hydrobromide | EC50 | 9.00E−05 |
| Adrenergic Receptor α₁A | ADRA1A | Antagonist | Tamsulosin | IC50 | 0.00115 |
| Adrenergic Receptor α₂A | ADRA2A | Agonist | UK 14304 | EC50 | 6.00E−05 |
| Adrenergic Receptor α₂A | ADRA2A | Antagonist | Yohimbine | IC50 | 0.00463 |
| Adrenergic Receptor β1 | ADRB1 | Agonist | (−)-Isoproterenol | EC50 | 0.002 |
| Adrenergic Receptor β1 | ADRB1 | Antagonist | Betaxolol | IC50 | 0.0034 |
| Adrenergic Receptor β2 | ADRB2 | Agonist | (−)-Isoproterenol | EC50 | 3.00E−04 |
| Adrenergic Receptor β2 | ADRB2 | Antagonist | ICI 118,551 | IC50 | 0.00056 |
| | | | hydrochloride | | |
| Nuclear Hormone Androgen | AR | Agonist | 6a-Fluorotestosterone | EC50 | 0.00195 |
| Receptor | | | | | |
| Nuclear Hormone Androgen | AR | Antagonist | Geldanamycin | IC50 | 0.09429 |
| Receptor | | | | | |
| Arginine Vasopressin | AVPR1A | Agonist | [Arg⁸]-Vasopressin | EC50 | 0.00037 |
| Receptor 1A | | | | | |
| Arginine Vasopressin | AVPR1A | Antagonist | SR 49059 | IC50 | 0.00182 |
| Receptor 1A | | | | | |
| Voltage-gated L-type calcium | CAV1.2 | Blocker | Isradipine | IC50 | 0.01691 |
| channel | | | | | |
| Cholecystokinin Receptor A | CCKAR | Agonist | (Tyr[SO₃H]₂₇)Cholecysto- | EC50 | 1.00E−04 |
| | | | kinin fragment 26-33 | | |
| | | | Amide | | |
| Cholecystokinin Receptor A | CCKAR | Antagonist | SR 27897 | IC50 | 0.03707 |
| Muscarinic acetylcholine | CHRM1 | Agonist | Acetylcholine chloride | EC50 | 0.01621 |
| Receptor M1 | | | | | |
| Muscarinic acetylcholine | CHRM1 | Antagonist | Atropine | IC50 | 0.00306 |
| Receptor M1 | | | | | |
| Muscarinic acetylcholine | CHRM2 | Agonist | Acetylcholine chloride | EC50 | 0.02486 |
| Receptor M2 | | | | | |
| Muscarinic acetylcholine | CHRM2 | Antagonist | Atropine | IC50 | 0.00406 |
| Receptor M2 | | | | | |
| Muscarinic acetylcholine | CHRM3 | Agonist | Acetylcholine chloride | EC50 | 0.03952 |
| Receptor M3 | | | | | |
| Muscarinic acetylcholine | CHRM3 | Antagonist | Atropine | IC50 | 0.0015 |
| Receptor M3 | | | | | |
| Cannabinoid Receptor 1 | CNR1 | Agonist | CP 55940 | EC50 | 4.00E−05 |
| Cannabinoid Receptor 1 | CNR1 | Antagonist | AM 251 | IC50 | 0.00324 |
| Cannabinoid Receptor 2 | CNR2 | Agonist | CP 55940 | EC50 | 0.00016 |
| Cannabinoid Receptor 2 | CNR2 | Antagonist | SR 144528 | IC50 | 0.03516 |
| Cyclooxygenase 1 | COX1 | Inhibitor | Indomethacin | IC50 | 0.0329 |
| Cyclooxygenase 2 | COX2 | Inhibitor | NS-398 | IC50 | 0.02245 |
| Dopamine transporter | DAT | Blocker | GBR 12909 | IC50 | 0.01456 |
| Dopamine Receptor D1 | DRD1 | Agonist | Dopamine | EC50 | 0.0855 |
| Dopamine Receptor D1 | DRD1 | Antagonist | SCH 39166 | IC50 | 0.00092 |
| Dopamine Receptor D2 | DRD2S | Agonist | Dopamine | EC50 | 0.001 |
| Dopamine Receptor D2 | DRD2S | Antagonist | Risperidone | IC50 | 0.00158 |
| Endothelin Receptor Type A | EDNRA | Agonist | Endothelin 1 | EC50 | 0.0011 |
| Endothelin Receptor Type A | EDNRA | Antagonist | BMS 182874 | IC50 | 1.10701 |
| Gamma-aminobutyric acid | GABAA | Blocker | Picrotoxin | IC50 | 2.77847 |
| Receptor A | | | | | |
| Gamma-aminobutyric acid | GABAA | Opener | GABA | EC50 | 6.35785 |
| Receptor A | | | | | |
| Nuclear Hormone | GR | Agonist | Dexamethasone | EC50 | 0.04971 |
| Glucocorticoid Receptor | | | | | |
| Nuclear Hormone | GR | Antagonist | Mifepristone | IC50 | 0.07236 |
| Glucocorticoid Receptor | | | | | |
| Nuclear Hormone | HERG | Blocker | Astemizole | IC50 | 0.22171 |
| Glucocorticoid Receptor | | | | | |
| Histamine Receptor H1 | HRH1 | Agonist | Histamine | EC50 | 0.03202 |
| Histamine Receptor H1 | HRH1 | Antagonist | Mepyramine | IC50 | 0.00538 |
| Histamine Receptor H2 | HRH2 | Agonist | Histamine | EC50 | 0.26388 |
| Histamine Receptor H2 | HRH2 | Antagonist | Tiotidine | IC50 | 0.13915 |
| Insulin Receptor (tyrosine | INSR | Inhibitor | BMS-754807 | IC50 | 0.00052 |
| kinase) | | | | | |
| Kv11.1, the alpha subunit of a | KvLQT1/minK | Blocker | XE 991 | IC50 | 1.66819 |
| potassium ion channel | | | | | |
| Kv11.1, the alpha subunit of a | KvLQT1/mink | Opener | ML-277 | EC50 | 2.30579 |
| potassium ion channel | | | | | |
| Lymphocyte Cell-Specific | LCK | Inhibitor | Gleevec | IC50 | 13.36093 |
| Protein-Tyrosine Kinase (Src | | | | | |
| family) | | | | | |
| Monoamine oxidase type A | MAOA | Inhibitor | Clorgyline | IC50 | 0.00446 |
| Nicotinic acetylcholine | nACHR(a4/b2) | Blocker | Dihydro-AY-erythroidine | IC50 | 0.68211 |
| Receptor α4 β2 | | | | | |
| Nicotinic acetylcholine | nACHR(a4/b2) | Opener | (−)-Nicotine | EC50 | 2.3741 |
| Receptor α4 β2 | | | | | |
| A tetrodotoxin-resistant | NAV1.5 | Blocker | Lidocaine | IC50 | 32.04972 |
| voltage-gated sodium channel | | | | | |
| N-methyl-D-aspartate | | | | | |
| (NMDA) Glutamate | | | | | |
| Norepinephrine transporter | NET | Blocker | Desipramine | IC50 | 0.01292 |
| N-methyl-D-aspartate | NMDAR | Blocker | (+)-MK 801 maleate | IC50 | 0.03884 |
| (NMDA) Glutamate Receptor | (1A/2B) | | | | |
| 1A/2B | | | | | |
| N-methyl-D-aspartate | NMDAR | Opener | L-Glutamic Acid | EC50 | 0.413 |
| (NMDA) Glutamate Receptor | (1A/2B) | | | | |
| 1A/2B | | | | | |
| Opioid Receptor Delta 1 | OPRD1 | Agonist | DADLE | EC50 | 0.00012 |
| Opioid Receptor Delta 1 | OPRD1 | Antagonist | Naltriben | IC50 | 0.00039 |
| Opioid Receptor Kappa 1 | OPRK1 | Agonist | Dynorphin A (1-17) | EC50 | 0.01234 |
| Opioid Receptor Kappa 1 | OPRK1 | Antagonist | nor-Binaltorphimine | IC50 | 0.00724 |
| Opioid Receptor Mu | OPRM1 | Agonist | DAMGO | EC50 | 0.00221 |
| Opioid Receptor Mu | OPRM1 | Antagonist | Naloxone | IC50 | 0.00552 |
| cGMP-inhibited cyclic | PDE3A | Inhibitor | Cilostamide | IC50 | 0.05554 |
| nucleotide phosphodiesterase | | | | | |
| 3A | | | | | |
| CAMP-specific 3′,5′-cyclic | PDE4D2 | Inhibitor | Cilomilast | IC50 | 0.01688 |
| phospho-diesterase | | | | | |
| Catecholamine Transporters | | | | | |
| Rho Associated Coiled-Coil | ROCK1 | Inhibitor | Staurosporine | IC50 | 8.00E−05 |
| Containing Protein Kinase 1 | | | | | |
| (serine-threonine kinase) | | | | | |
| Serotonin transporter | SERT | Blocker | Clomipramine | IC50 | 0.00242 |
| Vascular endothelial growth | VEGFR2 | Inhibitor | SU-11248 | IC50 | 0.00022 |
| factor receptor 2 (KDR | | | | | |
| tyrosine kinase) | | | | | |

### Example 12: Human Monoamine Transporter (hMAT) Release Assays

To assess the effects of indolizine derivatives on extracellular dopamine and serotonin concentrations, in vitro measures of serotonin and dopamine release were made using Chinese hamster ovary cells that stably expressed human monoamine transporters, dopamine (hDAT) and serotonin (hSERT) transporter. Dextroamphetamine and norfenfluramine were used as reference releasers of dopamine and serotonin, respectively.

Assay results revealed that 1-(indolizin-3-yl)-2-(methylamino)propan-1-one was more potent at releasing DAT than 5-HT, with a DAT/SERT ratio suggesting MDMA-like effects.

TABLE 4
Effects of Structure XLX on neurotransmitter
release from DAT and SERT

| | EC₅₀ DAT | EC₅₀ SERT | DAT/SERT |
|---|---|---|---|
| | (nM) | (nM) | ratio* |
| 1-(indolizin-3-y1)-2- | 227 | 109 | 2.1 |
| (methylamino)propan-1-one | | | |

*DAT/SERT ratios are calculated here as (DAT EC₅₀)⁻¹/(SERT EC₅₀)⁻¹ where larger numbers indicate higher DAT selectivity

hSERT Release Measurement Methods

Chinese hamster ovary cells expressing human SERT were seeded in Cytostar™ (PerkinElmer) plate with standard culture medium the day before the experiment at a single density (5 000 cells/assay). Cells were incubated overnight with 5% CO₂ at 37° C. The day of experiment, the medium was replaced by incubation buffer (140 mM NaCl, 4.8 mM KCl, 1.2 mM MgSO4, 0.1 mM KH₂PO₄, 10 mM HEPES, pH 7.4) with a single concentration of [³H]serotonin at 150 nM. Experiments comparing release in radioligand-free incubation buffer versus incubation buffer containing [³H]serotonin determined that the latter provided better signal stability. Therefore, this was used for experiments.

In control wells, the specificity of hSERT uptake was verified by adding the reference control imipramine (100 μM).

Two control conditions were used: (1) buffer only (with 1% DMSO concentration to match that in the test compound condition) to verify the background level of release; and (2) one reference SERT substrate compound, norfenfluramine, at 100 μM, to make it possible to calculate a relative Emax. Pilot studies varying DMSO concentration from 0.1 to 3% indicated that signal decreased at higher DMSO concentrations but that 1% DMSO retained good properties.

Cells were incubated at room temperature at different incubation times and radioactivity counted. Test compounds were measured at concentrations of 1e-10, 1e-09, 1e-08, 1e-07, 1e-06, 1e-05, and 1e-04 M. Each experiment was performed in duplicate (n=2).

hDAT Release Measurement Methods

Chinese hamster ovary cells expressing human DAT were seeded in Cytostar™ plate with standard culture medium the day before experiment at one single density (2 500 cells/assay). Cells were incubated overnight with 5% CO₂ at 37° C. The day of experiment, the medium was replaced by incubation buffer (TrisHCl 5 mM, 120 mM NaCl, 5.4 mM KCl, 1.2 mM MgSO₄, 1.2 mM CaCl₂), Glucose 5 mM, 7.5 mM HEPES, pH 7.4) with a single concentration of [3H]dopamine at 300 nM. Experiments comparing release in radioligand-free incubation buffer versus incubation buffer containing [³H]dopamine determined that the latter provided better signal stability. Therefore, this was used for experiments.

For all assays, three reference conditions were employed: (1) radioligand-containing buffer only, to verify the control level of release, (2) buffer with 1% DMSO (solvent used to solubilize the test compounds), (3) 100 uM amphetamine (in 1% DMSO) to make it possible to calculate a relative Emax.

Cells were incubated at room temperature at different incubation times and radioactivity counted. Test compounds were measured at concentrations of 1e-10, 1e-09, 1e-08, 1e-07, 1e-06, 1e-05, and 1e-04 M. Each experiment was performed in duplicate (n=2).

### Statistical Analysis

EC/IC50s were calculated using the R package drc (to fit the regression model) and LL.4 (to define the structure of the log-logistic regression model). Values were fit to the following function:

$$f ( x ) = c + ( d - c ) / ( 1 + exp ( b ( log ( x ) - log ( e ) ) ) where b = the Hill coefficent , \\ c = minimum value , d = maximum value , and e = E {C}_{50} / {IC}_{50} .$$

Values were calculated for both experimental repetitions at both stable inhibition times (60 and 90 minutes), resulting in four estimates of EC50 and other parameters for each compound and transporter. These four values were averaged to produce final estimates for each compound and transporter.

### Example 13: Enantiomeric Separation of Racemic Compounds of the Present Invention

Enantiomers of the present invention can be separated as described herein in the presence or absence of a protecting group. For example, when a compound of the present invention has an amino or hydroxyl substituent a chiral auxiliary or achiral protecting group can be installed on the amino or hydroxyl substituent to facilitate separation or enrichment of its enantiomers. This group can then be removed using conventional methods after separation.

### Example 14: Serum Serotonin Concentrations to Index Drug Interactions with the Serotonin Transporter (SERT, SLC6A4)

Serum serotonin can be measured using High Performance Liquid Chromatography and Fluorescence Detection. Venipuncture collects at least 1 mL of sample, which is spun with serum frozen to below −20° C. within 2 hours of collection. For active compounds, assay results will show increases in serum serotonin, indicating that the compound is a releaser of serotonin.

### Example 15: Human Serotonin Transporter (SERT, SLC6A4) Functional Antagonist Uptake Assay

Human recombinant serotonin transporter expressed in HEK-293 cells are plated. Test compound and/or vehicle is preincubated with cells (1×10E5/ml) in modified Tris-HEPES buffer pH 7.1 for 20 minutes at 25° C. and 65 nM. [3H]Serotonin is then added for an additional 15-minute incubation period. Bound cells are filtered and counted to determine [3H]Serotonin uptake. Compounds are screened at concentrations from 10 to 0.001 μM or similar. Reduction of [3H]Serotonin uptake relative to 1 μM fluoxetine indicates inhibitory activity.

### Example 16: Monoamine Transporter Uptake and Release Assays

An alternative, invasive method of measuring compound interactions with the serotonin, dopamine, or norepinephrine transporter can be conducted according to the methods of Solis et al (2017. Neuropsychopharmacology, 42(10), 1950-1961) and Rothman and Baumann (Partilla et al. 2016. In: Bönisch S, Sitte H H (eds) Neurotransmitter Transporters Springer; New York, pp 41-52).

Male Sprague-Dawley rats (Charles River, Kingston, NY, USA) are used for the synaptosome assays. Rats are group-housed with free access to food and water, under a 12 h light/dark cycle with lights on at 0700 h. Rats are euthanized by CO2 narcosis, and synaptosomes prepared from brains using standard procedures (Rothman, R. B., & Baumann, M. H. (2003). Monoamine transporters and psychostimulant drugs. European journal of pharmacology, 479(1-3), 23-40). Transporter uptake and release assays are performed as described previously (Solis et al. (2017). N-Alkylated analogs of 4-methylamphetamine (4-MA) differentially affect monoamine transporters and abuse liability. Neuropsychopharmacology, 42(10), 1950-1961). In brief, synaptosomes are prepared from caudate tissue for dopamine transporter (DAT) assays, and from whole brain minus caudate and cerebellum for norepinephrine transporter (NET) and serotonin (5-HT) transporter (SERT) assays.

For uptake inhibition assays, 5 nM [3H]dopamine, [3H]norepinephrine, or [3H]5-HT are used for DAT, NET, or SERT assays respectively. To optimize uptake for a single transporter, unlabeled blockers are included to prevent the uptake of [3H]transmitter by competing transporters. Uptake inhibition is initiated by incubating synaptosomes with various doses of test compound and [3H]transmitter in Krebs-phosphate buffer. Uptake assays were terminated by rapid vacuum filtration and retained radioactivity is quantified with liquid scintillation counting (Baumann et al. (2013). Powerful cocaine-like actions of 3, 4-methylenedioxypyrovalerone (MDPV), a principal constituent of psychoactive ‘bath salts’ products. Neuropsychopharmacology, 38(4), 552-562).

For release assays, 9 nM [3H]MPP+ is used as the radiolabeled substrate for DAT and NET, whereas 5 nM [3H]5-HT is used for SERT. Alternatively [3H]dopamine and [3H]norepinephrine may be used for DAT and NET assays, respectively. All buffers used in the release assay contain 1 μM reserpine to block vesicular uptake of substrates. The selectivity of release assays is optimized for a single transporter by including unlabeled blockers to prevent the uptake of [3H]MPP+ or [3H]5-HT by competing transporters. Synaptosomes are preloaded with radiolabeled substrate in Krebs-phosphate buffer for 1 h to reach steady state. Release assays are initiated by incubating preloaded synaptosomes with various concentrations of the test drug. Release is terminated by vacuum filtration and retained radioactivity quantified by liquid scintillation counting.

Effects of test drugs on release are expressed as a percent of maximal release, with maximal release (i.e., 100% Emax) defined as the release produced by tyramine at doses that evoke the efflux of all ‘releasable’ tritium by synaptosomes (10 μM tyramine for DAT and NET assay conditions, and 100 μM tyramine for SERT assay conditions). Effects of test drugs on uptake inhibition and release are analyzed by nonlinear regression. Dose-response values for the uptake inhibition and release are fit to the equation, Y(x)=Ymin+(Ymax−Ymin)/(1+10 exp[(log P50−log x)]×n), where x is the concentration of the compound tested, Y(x) is the response measured, Ymax is the maximal response, P50 is either IC50 (the concentration that yields half-maximal uptake inhibition response) or EC50 (the concentration that yields half-maximal release), and n is the Hill slope parameter. EC50s for release of less than 10 uM, but often less than 1 uM, are usually considered indicative of substrate-type releasers.

### Example 17: Marble Burying Measure of Decreased Anxiety and Neuroticism

The marble burying test is a model of neophobia, anxiety, and obsessive-compulsive behavior. Moreover, it has been proposed to have predictive validity for the screening of novel antidepressants and anxiolytics. It is well established to be sensitive to the effects of SSRIs as well as serotonin releasers such as fenfluramine and MDMA (De Brouwer et al., Cognitive, Affective, and Behavioral Neuroscience, 2019, 19(1), 1-39).

The test involves the placement of a standardized number of marbles gently onto the surface of a layer of bedding material within a testing arena. Mice are then introduced into the arena for a standardized amount of time and allowed to explore the environment. The outcome measure of the test is the number of marbles covered, as scored by automatic scoring software or blinded observers. General locomotor activity, often operationalized as total distance traveled, is often used as a control measure. A compound that attenuates anxiety, neuroticism, or obsessive-compulsive behavior decreases marble burying. A compound of the present invention is given to mice and decreases in marble burying, indicates an acute decrease in anxiety and neuroticism.

### Example 18: Neuroplasticity Assay in Primary Cortical Neurons

Compounds of the current invention can be considered psychoplastogens, that is, small molecules that are able to induce rapid neuroplasticity (Olson, 2018, Journal of experimental neuroscience, 12, 1179069518800508). One exemplary method for measuring this, a neurite outgrowth assay conducted in murine primary cortical neurons, is provided below. Other methods are well known in the literature (e.g., Olson, 2018, Journal of experimental neuroscience, 12, 1179069518800508; Ly et al. Cell reports 23, no. 11 (2018): 3170-3182; and references therein).

Primary cortical neurons are prepared from timed pregnant wild-type C57BL/6JRccHsd mice at E18. Animals are sacrificed (see section 3.3.1) and embryos are dissected in Calcium and Magnesium free Hanks Balanced Salt Solution (CMF-HBSS) containing 15 mM HEPES and 10 mM NaHCO3, pH 7.2. Embryos are decapitated, skin and skull gently removed and hemispheres are separated. After removing meninges and brain stem, the hippocampi are isolated, chopped with a sterile razor blade in Chop solution (Hibernate-E without Calcium containing 2% B-27) and digested in 2 mg/mL papain (Worthington) dissolved in Hibernate-E without Calcium for 30 minutes (±5 min) at 30° C. Hippocampi are triturated for 10-15 times with a fire-polished silanized Pasteur pipette in Hibernate-E without Calcium containing 2% B-27, 0.01% DNaseI, 1 mg/mL BSA, and 1 mg/mL Ovomucoid Inhibitor. Undispersed pieces are allowed to settle by gravity for 1 min and the supernatant is centrifuged for 3 min at 228 g. The pellet is resuspended in Hibernate-E containing 2% B-27, 0.01% DNaseI, 1 mg/ml BSA, 1 mg/mL Ovomucoid Inhibitor and diluted with Hibernate-E containing 2% B-27. After the second centrifugation step (3 min at 228 g), the pellet is resuspended in nutrition medium (Neurobasal, 2% B-27, 0.5 mM glutamine, 1% Penicillin-Streptomycin).

Cells are counted in a hemacytometer and seeded in nutrition medium on poly-D-lysine pre-coated 96-well plates at a density of 2.6×104 cells/well. Cells are cultured at 37° C.; 95% humidity and 5% CO2. All wells are handled the same way.

The experiment is performed in adequate technical replicates for all groups, for example five replicates.

On the day of preparation (DIV1), mouse cortical neurons are seeded on poly-D-lysine pre-coated 96-well plates at a density of 2.6×104 cells per well.

On DIV2, cells are treated with test compounds at concentrations selected based on their EC50 at SERT release or 5-HT receptor agonism for three different time points (4 h, 8 h and 24 h), followed by a complete medium change. Additionally, cells are treated with 40 ng/mL of a positive control (Fibroblast growth factor, FGF) or vehicle control (VC) for 48 h.

The experiment is carried out with several, for example five, technical replicates per condition, vehicle treated cells serve as control.

Treated primary neurons are fixed on DIV4 by addition of equal volume 4% paraformaldehyde (PFA) to the medium at room temperature (RT) for 30 minutes.

Cells are rinsed two times with PBS and are permeabilized with 0.1% Triton X-100 in PBS for 30 minutes at RT. Next, cells are blocked for 90 min at RT with 20% horse serum, 0.1% Triton X-100 in PBS.

Then, samples are incubated with the primary antibody against Beta Tubulin Isotype III at 4° C. overnight.

Next day, cells are further incubated for another 30 min at RT. After three washing steps with PBS, cells are incubated with a fluorescently labelled secondary antibody and DAPI (nucleus) for 1.5 hours at RT in the darkness. Cells are again rinsed four times with PBS and imaged with the Cytation 5 Multimode reader (BioTek). From each well, images are taken at 10× magnification.

Digital images from cortical neurons are analyzed for the following parameter using a software-supported automatic quantification method: Number of neurites, number of branches, total length of neurites and length of the longest neurite. Analysis is performed using HCA-Vision software or similar standard software.

Basic statistical analysis is performed. If appropriate, data are presented as mean±standard error of mean (SEM) and group differences are evaluated by e.g., one or two-way ANOVA or T-test. EC50 may be calculated as described elsewhere.

### Example 19: Evaluation of Entactogenic Effect of Decreased Neuroticism

The entactogenic effect of decreased neuroticism can be measured as a decrease in social anxiety using the Brief Fear of Negative Evaluation-revised (BFNE) (Carleton et al., 2006, Depression and Anxiety, 23(5), 297-303; Leary, 1983, Personality and Social Psychology bulletin, 9(3), 371-375). This 12-item Likert scale questionnaire measures apprehension and distress due to concerns about being judged disparagingly or with hostility by others. Ratings use a five-point Likert scale with the lowest, middle, and highest values labeled with “much less than normal,” “normal,” and “much more than normal.” The BFNE can be administered before and repeatedly during therapeutic drug effects. Participants are instructed to answer how they have been feeling for the past hour, or otherwise during the effect of the drug. Baseline-subtracted responses are typically used in statistical models.

### Example 20: Evaluation of Entactogenic Effect of Authenticity

The entactogenic effect of authenticity can be measured using the Authenticity Inventory (Kernis & Goldman. 2006. Advances in experimental social psychology, 38, 283-357) as modified by Baggott et al (Journal of Psychopharmacology 2016, 30.4: 378-87). Administration and scoring of the instrument is almost identical to that of the BFNE. The Authenticity Inventory consists of the following items, which are each rated on a 1-5 scale, with select items reverse scored as specified by Kernis & Goldman:
I am confused about my feelings.
I feel that I would pretend to enjoy something when in actuality I really didn't.
For better or worse, I am aware of who I truly am.
I understand why I believe the things I do about myself
I want the people with whom I am close to understand my strengths.
I actively understand which of my self-aspects fit together to form my core or true self.
I am very uncomfortable objectively considering my limitations and shortcomings.
I feel that I would use my silence or head-nodding to convey agreement with someone else's statement or position even though I really disagreed.
I have a very good understanding of why I do the things I do.
I am willing to change myself for others if the reward is desirable enough.
I would find it easy to pretend to be something other than my true self.
I want people with whom I am close to understand my weaknesses.
I find it difficult to critically assess myself. (unchanged)
I am not in touch with my deepest thoughts and feelings.
I feel that I would make it a point to express to those I am close with how much I truly care for them.
I have difficulty accepting my personal faults, so I try to cast them in a more positive way.
I feel that I idealize the people close to me rather than objectively see them as they truly are.
If asked, people I am close to could accurately describe what kind of person I am.
I prefer to ignore my darkest thoughts and feelings.
I am aware of times when I am not being my true self.
I am able to distinguish the self-aspects that are important to my core or true self from those that are unimportant.
People close to me would be shocked or surprised if they discovered what I am keeping inside me.
It is important for me to understand the needs and desires of those with whom I am close.
I want people close to me to understand the real me, rather than just my public persona or “image”.
I could act in a manner that is consistent with my personally held values, even if others criticized me or rejected me for doing so.
If a close other and I were in disagreement, I would rather ignore the issue than constructively work it out.
I feel that I would do things that I don't want to do merely to avoid disappointing people.
My behavior expresses my values.
I actively attempt to understand myself as well as possible.
I feel that I'd rather feel good about myself than objectively assess my personal limitations and shortcomings.
My behavior expresses my personal needs and desires.
I have on a “false face” for others to see.
I feel that I would spend a lot of energy pursuing goals that are very important to other people even though they are unimportant to me.
I am not in touch with what is important to me.
I try to block out any unpleasant feelings I have about myself.
I question whether I really know what I want to accomplish in my lifetime.
I am overly critical about myself.
I am in touch with my motives and desires.
I feel that I would deny the validity of any compliments that I receive.
I place a good deal of importance on people close to me understanding who I truly am.
I find it difficult to embrace and feel good about the things I have accomplished.
If someone pointed out or focused on one of my shortcomings, I would quickly try to block it out of my mind and forget it.
The people close to me could count on me being who I am, regardless of what setting we were in.
My openness and honesty in close relationships are extremely important to me.
I am willing to endure negative consequences by expressing my true beliefs about things.

### Example 21: Evaluation of Side Effects of Entactogens

Adverse effects of an entactogen include formation of tolerance to entactogens, headache, difficulty concentrating, lack of appetite, lack of energy, and decreased mood. In addition to these mild toxicities, MDMA is associated with a number of more severe toxicities, including but not limited to acute and chronic cardiovascular changes, hepatotoxicity, hyperthermic syndromes, hyponatremia, and neurotoxicity (see the MDMA Investigator's Brochure, 13th Edition: Mar. 22, 2021, and references therein, available from the sponsor of MDMA clinical trials at MAPS.org).

Acute physiological changes can be measured in humans with standard clinical methods (blood pressure cuffs, 3-lead EKG, tympanic or oral temperature, serum sodium, etc), with measures usually collected before and at scheduled intervals after an entactogen. For example, measures may be collected before, 0.5, 0.75, 1, 1.5, 2, 2.5, 3, 4, 5, 6, and 8 hours after an entactogen. Maximum change from baseline and area-under-the-effects-versus-time-curve may be used as summary measures and statistically compared to a placebo control condition.

To measure adverse symptoms, patients can be asked to complete a self-report symptom questionnaire, such as the Subjective Drug Effects Questionnaire (SDEQ) or List of Complaints. The SDEQ is a 272-item self-report instrument measuring perceptual, mood, and somatic changes caused by drugs including hallucinogens like LSD (Katz et al. 1968. J Abnorm Psychology 73:1-14). It has also been used to measure the therapeutic and adverse effects of MDMA (Harris et al. 2002. Psychopharmacology, 162(4), 396-405). The List of Complaints is a 66-item questionnaire that measures physical and general discomfort and is sensitive to entactogen-related complaints (e.g., Vizeli & Liechti. 2017. Journal of Psychopharmacology, 31(5), 576-588).

Alternatively, individual items can be taken from the SDEQ or List of Complaints in order to create more focused questionnaires and reduce the burden of filling out time-consuming paperwork on participants. To measure tolerance formation, a global measure of the intensity of therapeutic effects can be used, such as the question “on a scale from 0 to 100 where 0 is no ‘good drug effect’ and 100 is the most ‘good drug effect’ you have ever felt, how would you rate this drug experience?”

In some embodiments, the questionnaire will be administered approximately 7 hours after a patient takes MDMA or another entactogen (with instructions to answer for the time since taking the entactogen) and then daily (with instructions to answer for the last 24 hours) for up to 96 hours after the entactogen was taken. Decreases in adverse effects of a compound compared to MDMA can be shown by comparing the intensity (for the tolerance question) or prevalence (for other symptom questions) of effects that occur. Prevalence of adverse effects including formation of tolerance to entactogens, headache, difficulty concentrating, lack of appetite, lack of energy, and decreased mood may be decreased by approximately 10%, 20%, 30%, 40%, 50%, 60%, 70%, 80%, 90%, 99%, or 100%.

As an alternative to measuring side effects of entactogens in clinical trials, preclinical studies in rodents may also be used. Appropriate tasks and behaviors that may be used to measure side effects include physiological measures (heart rate, blood pressure, body temperature), the modified Irwin procedure or functional observational battery (Irwin, Psychopharmacologia, 13, 222-257, 1968), and locomotor activity (such as distance traveled, rearing frequency, and rearing duration; Piper et la., J Pharmacol Exp Ther, 317, 838-849, 2006). In these studies, an entactogen is administered at different doses (including a vehicle only placebo) to different groups of animals and measures are made at scheduled times before and after administration. For example, 0, 1.5, 3, 15, and 30 mg/kg of a compound may be administered intraperitoneally and measures made before and 15, 30, 60, 120 and 180 minutes and 12, 24, 36, and 48 hours after administration of the test substance.

While the present invention is described in terms of particular embodiments and applications, it is not intended that these descriptions in any way limit its scope to any such embodiments and applications, and it will be understood that many modifications, substitutions, changes, and variations in the described embodiments, applications, and details of the invention illustrated herein can be made by those skilled in the art without departing from the spirit of the invention, or the scope of the invention as described in the appended claims.

I claim:

1. A compound, enantiomerically enriched mixture, or a pure enantiomer of formula: 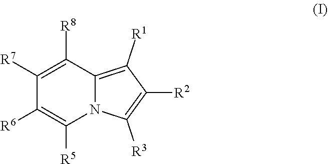
or a pharmaceutically acceptable salt or salt mixture thereof;
wherein:
R² is selected from the group consisting of R¹, 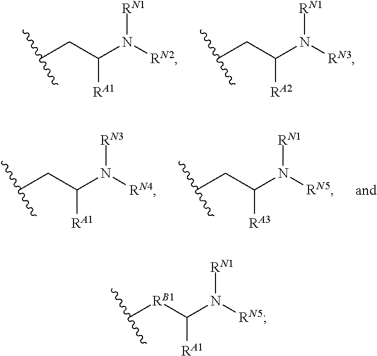
R³ is selected from the group consisting of R¹, 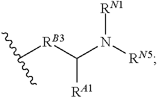
R⁵, R⁶, R⁷, and R⁸ are independently selected from the group consisting of R¹ and 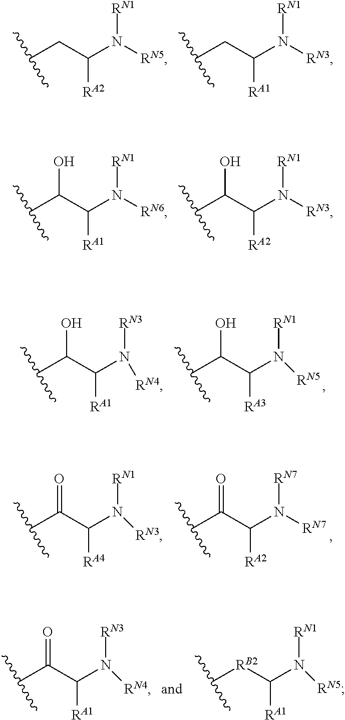
wherein 5 of the 6 of R², R³, R⁵, R⁶, R⁷, and R⁸ are R¹;
each R¹ is independently selected from the group consisting of hydrogen, halogen, alkyl, haloalkyl, —OP(O)(OR⁹)₂, —SR⁹, —NR⁹R¹⁰, or —OR⁹;
each R⁹ and R¹⁰ is independently selected from the group consisting of hydrogen, alkyl, and haloalkyl;
R^{A1} is hydrogen, —CH₃, —CH₂X, —CHX₂, —CX₃, —CH₂CH₃, —CH₂CH₂X, —CH₂CHX₂, —CH₂CX₃, —CH₂OH, or —CH₂CH₂OH;
R^{A2} is —CH₃, —CH₂X, —CHX₂, —CX₃, —CH₂CH₃, —CH₂CH₂X, —CH₂CHX₂, —CH₂CX₃, —CH₂OH, or —CH₂CH₂OH;
R^{A3} is —CH₂X, —CHX₂, —CX₃, —CH₂CH₃, —CH₂CH₂X, —CH₂CHX₂, —CH₂CX₃, —CH₂OH, or —CH₂CH₂OH;
R^{A4} is hydrogen, —CH₂X, —CHX₂, —CX₃, —CH₂CH₃, —CH₂CH₂X, —CH₂CHX₂, —CH₂CX₃, —CH₂OH, or —CH₂CH₂OH;
R^{B1} is 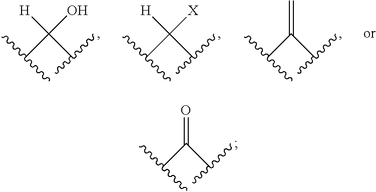
R^{B2} is 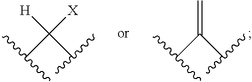
R^{B3} is 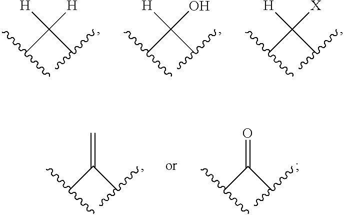
R^{N1} is hydrogen, —(C₁-C₆)alkyl, —CH₂CH₂X, —CH₂CHX₂, —CH₂CX₃, or —CH₂CH₂OH;
R^{N2} is —(C₃-C₆)alkyl, —CH₂CH₂X, —CH₂CHX₂, —CH₂CX₃, —CH₂CH₂OH, or hydroxy;
R^{N3} is —(C₁-C₆)alkyl, —CH₂CH₂X, —CH₂CHX₂, —CH₂CX₃, —CH₂CH₂H, or hydroxy;
R^{N4} is —(C₁-C₆)alkyl, —CH₂CH₂X, —CH₂CHX₂, —CH₂CX₃, or —CH₂CH₂OH;
R^{N5} is hydrogen, —(C₁-C₆)alkyl, —CH₂CH₂X, —CH₂CHX₂, —CH₂CX₃, —CH₂CH₂OH, or hydroxy;
R^{N6} is —(C₂-C₆)alkyl, —CH₂CH₂X, —CH₂CHX₂, —CH₂CX₃, —CH₂CH₂OH, or hydroxy;
R^{N7} is hydrogen, —(C₂-C₆)alkyl, —CH₂CH₂X, —CH₂CHX₂, —CH₂CX₃, —CH₂CH₂OH, or hydroxy; and
each X is independently selected from the group consisting of —F, —Cl, —Br, and —I.

2. The compound of claim 1 wherein the compound is of formula 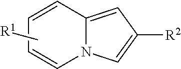
or a pharmaceutically acceptable salt thereof.

3. The compound of claim 2 wherein the compound is of formula 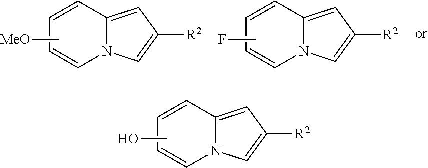
or a pharmaceutically acceptable salt thereof.

4. The compound of claim 1 wherein the compound is of formula 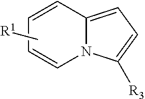
or a pharmaceutically acceptable salt thereof.

5. The compound of claim 1 wherein the compound is of formula 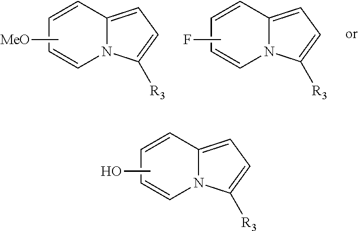
or a pharmaceutically acceptable salt thereof.

6. The compound of claim 1 wherein R³ is

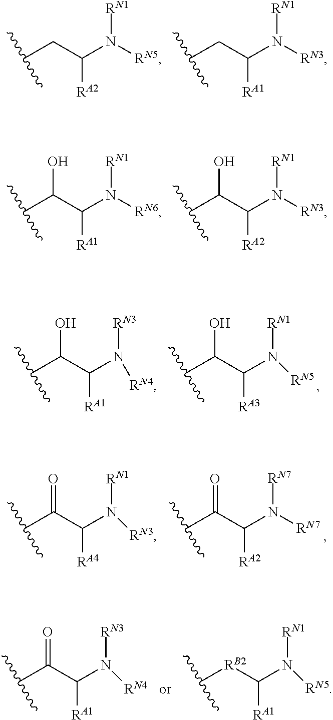

7. The compound of claim 1 wherein R² is

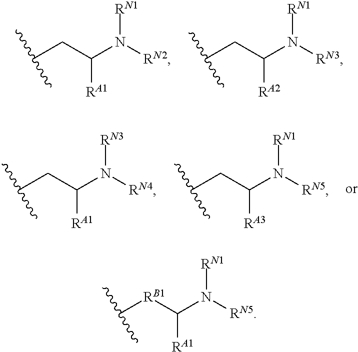

8. The compound of claim 1 wherein R¹ is hydrogen.

9. The compound of claim 1 wherein R¹ is halogen.

10. The compound of claim 1 wherein the compound is 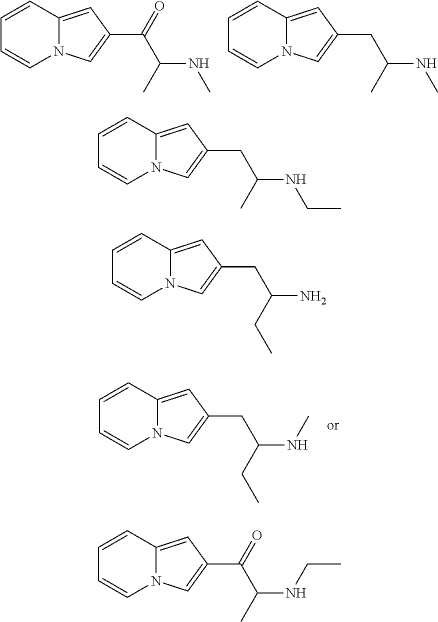
or a pharmaceutically acceptable salt or salt mixture thereof.

11. The compound of claim 1 wherein the compound is 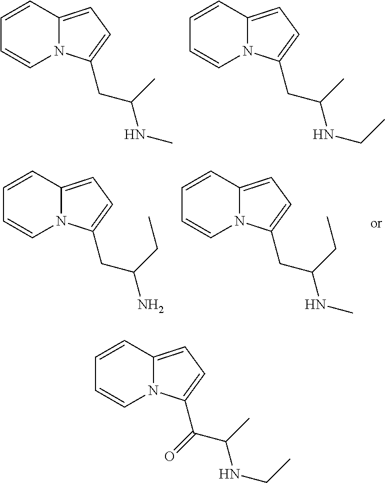
or a pharmaceutically acceptable salt or salt mixture thereof.

12. The compound of claim 1 wherein the compound is: 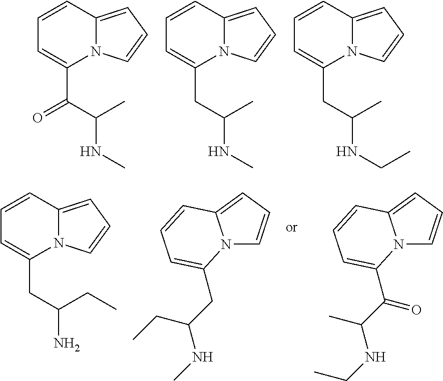
or a pharmaceutically acceptable salt or salt mixture thereof.

13. The compound of claim 1 wherein the compound is 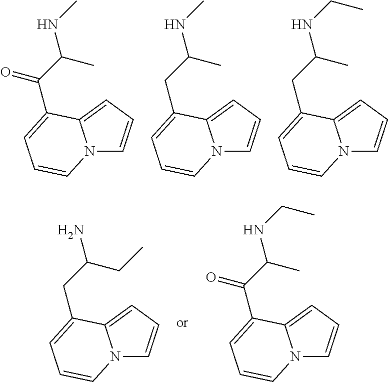
or a pharmaceutically acceptable salt or salt mixture thereof.

14. The compound of claim 1 wherein the compound is 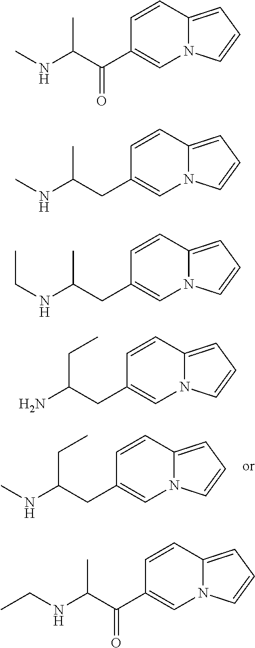
or a pharmaceutically acceptable salt or salt mixture thereof.

15. The compound of claim 1 wherein the compound is 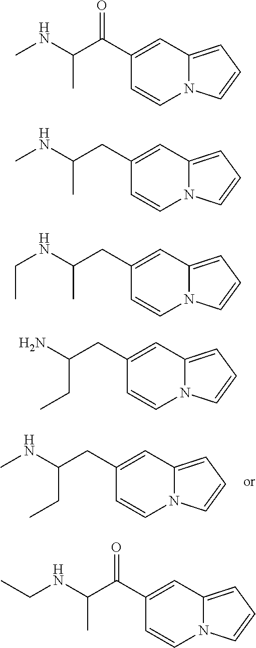
or a pharmaceutically acceptable salt or salt mixture thereof.

16. The compound of claim 1 wherein the compound is 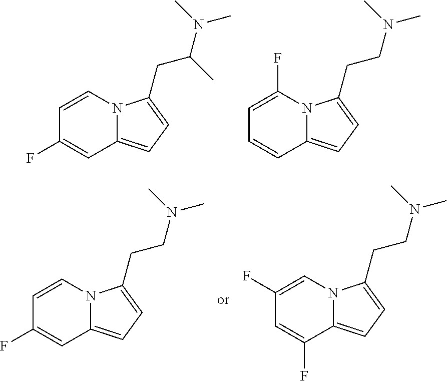
or a pharmaceutically acceptable salt or salt mixture thereof.

17. The compound of claim 1 wherein the compound is 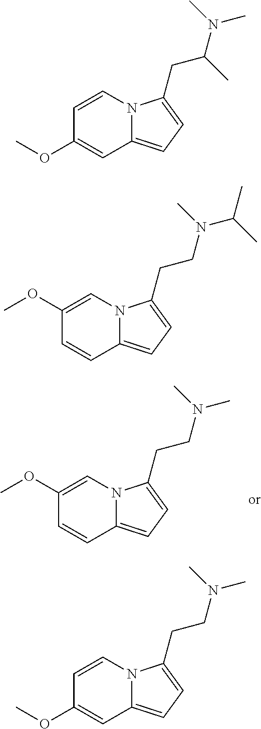
or a pharmaceutically acceptable salt or salt mixture thereof.

18. The compound of claim 1 wherein the compound is 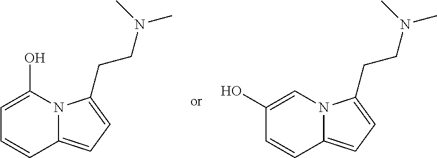
or a pharmaceutically acceptable salt or salt mixture thereof.

19. An enantiomerically enriched mixture of a compound of structure 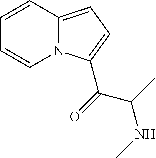
or a pharmaceutically acceptable salt or salt mixture thereof.

20. The compound of claim 1 wherein the pharmaceutically acceptable salt(s) is HCl, sulfate, aspartate, saccharate, fumarate, succinate, phosphate, oxalate, acetate, amino acid anion, gluconate, maleate, malate, citrate, mesylate, nitrate or tartrate, or a mixture thereof.

21. A pharmaceutical composition comprising a compound, an enantiomerically enriched mixture, or a pure enantiomer of claim 1, or a pharmaceutically acceptable salt or salt mixture thereof and a pharmaceutically acceptable excipient.

22. A method to treat a central nervous system disorder comprising administering an effective amount of a compound, enantiomerically enriched mixture, or a pure enantiomer of claim 1 to a patient in need thereof.

23. The method of claim 22, wherein the central nervous system disorder is selected from the group consisting of post-traumatic stress disorder, depression, dysthymia, anxiety, generalized anxiety, social anxiety, panic, adjustment disorder, feeding and eating disorders, binge behaviors, body dysmorphic syndromes, addiction, drug abuse or dependence disorders, substance use disorders, disruptive behavior disorders, impulse control disorders, gaming disorders, gambling disorders, memory loss, dementia of aging, attention deficit hyperactivity disorder, personality disorders, attachment disorders, autism, dissociative disorders, and headache disorders.

24. The method of claim 22, wherein the patient is a human.
